(19) United States
(12) Patent Application Publication
(10) Pub. No.: US 2012/0295923 A1
(43) Pub. Date: Nov. 22, 2012

(54) Aminocyclohexanes and Aminotetrahydropyrans and Related Compounds As Gamma-Secretase Modulators

(75) Inventors: Christopher William Am Ende, Mystic, CT (US); Benjamin Adam Fish, Groton, CT (US); Douglas Scott Johnson, Concord, MA (US); Ricardo Lira, Groton, CT (US); Christopher John O'Donnell, Mystic, CT (US); Martin Youngjin Pettersson, Littleton, MA (US); Corey Michael Stiff, Salem, CT (US)

(73) Assignee: PFIZER INC.

(21) Appl. No.: 13/575,370

(22) PCT Filed: Jan. 19, 2011
(86) PCT No.: PCT/IB2011/050240

§ 371 Date: Jul. 26, 2012

(30) Foreign Application Priority Data
Jan. 29, 2010 (US) 61299811

(51) Int. Cl. A61K 31/505; A61K 31/196; A61K 31/421; C07D 263/32; C07D 309/14; A61K 31/35; C07D 239/26; C07D 277/30; A61K 31/426; A61K 31/438; C07D 221/20; C07D 221/22; A61K 31/439; A61K 31/4433; C07D 405/04; C07D 213/55; A61K 31/4418; A61P 25/00; A61P 25/18; C07C 229/42

(52) U.S. Cl. 514/256; 562/456; 514/567; 514/374; 548/236; 549/424; 514/459; 544/242; 548/204; 514/365; 514/278; 546/16; 546/112; 514/299; 514/336; 546/282.1; 546/335; 514/357

(57) ABSTRACT

Compounds and pharmaceutically acceptable salts of the compounds are disclosed, wherein the compounds have the structure of Formula I

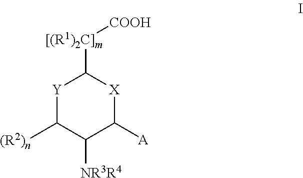

as defined in the specification. Corresponding pharmaceutical compositions, methods of treatment, methods of synthesis, and intermediates are also disclosed.

### FIELD OF THE INVENTION

The present invention relates to the treatment of Alzheimer's disease and other neurodegenerative and/or neurological disorders in mammals, including humans. This invention also relates to the modulation, in mammals, including humans, of the production of A-beta peptides that can contribute to the formation of neurological deposits of amyloid protein. More particularly, this invention relates to aminocyclohexane and aminotetrahydropyran compounds useful for the treatment of neurodegenerative and/or neurological disorders, such as Alzheimer's disease and Down's Syndrome, related to A-beta peptide production.

### BACKGROUND OF THE INVENTION

Dementia results from a wide variety of distinctive pathological processes. The most common pathological processes causing dementia are Alzheimer's disease (AD), cerebral amyloid angiopathy (CM) and prion-mediated diseases (see, e.g., Haan et al., Clin. Neurol. Neurosurg. 1990, 92(4):305-310; Glenner et al., J. Neurol. Sci. 1989, 94:1-28). AD affects nearly half of all people past the age of 85, the most rapidly growing portion of the United States population. As such, the number of AD patients in the United States is expected to increase from about 4 million to about 14 million by the middle of the next century. At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Several programs have been advanced by research groups to ameliorate the pathological processes causing dementia, AD, CM and prion-mediated diseases. γ-Secretase modulators are one such strategy and numerous compounds are under evaluation by pharmaceutical groups. The present invention relates to a group of brain penetrable γ-secretase modulators and as such are useful as γ-secretase modulators for the treatment of neurodegenerative and/or neurological disorders related to A-beta peptide production, such as Alzheimer's disease and Down's Syndrome. (see Olsen et al., Ann. Rep. Med. Chem. 2007, 42: 27-47).

### SUMMARY OF THE INVENTION

The present invention is directed to a compound, including the pharmaceutically acceptable salts thereof, having the structure of formula

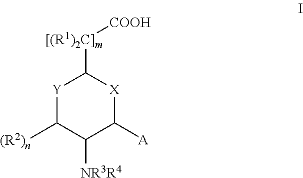

A is C₆₋₁₀aryl or 5- to 10-membered heteroaryl, optionally substituted with one to three R⁷;

X and Y are independently C(R⁹)₂, NR¹⁰ or O, wherein at least one of X or Y is C(R⁹)₂;

each R¹ is independently hydrogen, C₁₋₆alkyl or —(CH₂)ₜ—C₃₋₇cycloalkyl; or two R¹ substituents together with the carbon to which they are bonded can form a C₃₋₇cycloalkyl;

each R² is independently CF₃, fluorine, C₁₋₃alkyl, C₃₋₇cycloalkyl, or OR⁵, or two R² substituents together with the carbon to which they are bonded can form a C₃₋₄cycloalkyl;

R³ and R⁴ are independently C₁₋₆alkyl, C₃₋₇cycloalkyl, C₆₋₁₀aryl, 5- to 10-membered heteroaryl, or 4- to 10-membered heterocycloalkyl, wherein said alkyl, cycloalkyl, aryl, heteroaryl and heterocycloalkyl are optionally substituted by C₁₋₆alkyl, halogen, oxo, cyano, —CF₃, C₃₋₇cycloalkyl, C₆₋₁₀aryl, 5- to 10-membered heteroaryl, or 4- to 10-membered heterocycloalkyl, wherein said cycloalkyl, aryl, heterocycloalkyl, or heteroaryl substituents can be further substituted with one to three C₁₋₆alkyl, halogen, —(CH₂)ₜ—CF₃, hydroxy, oxo, or cyano;

alternatively, R³ and R⁴ together with the nitrogen to which they are bonded form a 4- to 10-membered heterocycloalkyl or 5- to 10-membered heteroaryl wherein said heterocycloalkyl or heteroaryl is optionally substituted with one to six R⁶ wherein two R⁶ together with the atom or atoms to which they are bonded, optionally including additional atoms of the heterocycloalkyl in the case of a bridged system, can form a C₃₋₇cycloalkyl, C₆₋₁₀aryl, 5- to 10-membered heteroaryl, or 4- to 10-membered heterocycloalkyl, wherein said cycloalkyl, aryl, heterocycloalkyl, or heteroaryl can be further substituted with one to three C₁₋₆alkyl, halogen, —CF₃, hydroxy, oxo, or cyano, and said C₁₋₆alkyl is optionally further substituted with one to three fluorine or —(CH₂)ₜ—CF₃;

each R⁵ is independently hydrogen or C₁₋₃alkyl, wherein said alkyl can be substituted with one to three fluorines;

each R⁶ is independently C₁₋₆alkyl, fluorine, —(CH₂)ₜ—CF₃, hydroxy, oxo, or cyano;

each R⁷ is independently —(CH₂)ₜ—CF₃, cyano, halogen, C₁₋₃alkyl, C₃₋₇cycloalkyl, or —OR⁸;

each R⁸ is independently hydrogen, C₁₋₃alkyl or —(CH₂)ₜ—CF₃;

each R⁹ is independently hydrogen, CF₃, fluorine, C₁₋₃alkyl, C₃₋₇cycloalkyl or OR⁵, or two R⁹ substituents together with the carbon to which they are bonded can form a C₃₋₄cycloalkyl;

R¹⁰ is hydrogen, —(CH₂)ₜ—CF₃, C₁₋₃alkyl or C₃₋₇cycloalkyl;

each n is an integer independently selected from 0, 1, or 2;

each m is an integer independently selected from 0, 1, or 2; and

each t is an integer independently selected from 0, 1, or 2.

In one embodiment of the invention, the so-called aminotetrahydropyrans, Y is C(R⁹)₂ wherein each R⁹ is hydrogen, and X is O; or pharmaceutically acceptable salt thereof.

In one embodiment of the invention, the so-called aminocyclohexanes, X and Y are C(R⁹)₂ wherein each R⁹ is hydrogen; or pharmaceutically acceptable salt thereof.

In one embodiment of the invention A is C₆₋₁₀aryl substituted with one R⁷; wherein R⁷ is —(CH₂)ₜ—CF₃, cyano, halogen, C₁₋₃alkyl, C₃₋₇cycloalkyl, or —OR⁸; or pharmaceutically acceptable salt thereof. In one embodiment of the invention A is C₆₋₁₀aryl substituted with one R⁷; wherein R⁷ is —(CH₂)ₜ—CF₃, cyano, or halogen; or pharmaceutically acceptable salt thereof. In another embodiment A is phenyl substituted with one R⁷ and R⁷ is —(CH₂)ₜ—CF₃, wherein t is zero; or pharmaceutically acceptable salt thereof. In another embodiment A is phenyl substituted with one R⁷ and R⁷ is cyano; or pharmaceutically acceptable salt thereof. In another embodiment A is phenyl substituted with one R⁷ and R⁷ is halogen; or pharmaceutically acceptable salt thereof. In another embodiment A is phenyl substituted with one R⁷ and R⁷ is chloro or fluoro; or pharmaceutically acceptable salt thereof.

In another embodiment of the invention A is C₆₋₁₀aryl substituted with two R⁷; wherein each R⁷ is independently —(CH₂)ₜ—CF₃, cyano, halogen, C₁₋₃alkyl, C₃₋₇cycloalkyl, or —OR⁸; or pharmaceutically acceptable salt thereof.

In another embodiment of the invention A is C₆₋₁₀ aryl substituted with three R⁷; wherein each R⁷ is independently —(CH₂)ₜ—CF₃, cyano, halogen, C₁₋₃alkyl, C₃₋₇cycloalkyl, or —OR⁸; or pharmaceutically acceptable salt thereof.

In one embodiment of the invention A is 5- to 10-membered heteroaryl substituted with one R⁷; wherein R⁷ is —(CH₂)ₜ—CF₃, cyano, halogen, C₁₋₃alkyl, C₃₋₇cycloalkyl, or —OR⁸; or pharmaceutically acceptable salt thereof. In another embodiment of the invention A is 5- to 10-membered heteroaryl substituted with one R⁷; wherein R⁷ is —(CH₂)ₜ—CF₃ or halogen; or pharmaceutically acceptable salt thereof. In another embodiment of the invention, A is pyridine substituted with one R⁷ and R⁷ is —(CH₂)ₜ—CF₃; or pharmaceutically acceptable salt thereof. In another embodiment A is pyridine substituted with one R⁷ and R⁷ is halogen; or pharmaceutically acceptable salt thereof. In another embodiment A is pyridine substituted with one R⁷ and R⁷ is chloro; or pharmaceutically acceptable salt thereof.

In another embodiment of the invention A is 5- to 10-membered heteroaryl substituted with two R⁷; wherein each R⁷ is independently —(CH₂)ₜ—CF₃, cyano, halogen, C₁₋₃alkyl, C₃₋₇cycloalkyl, or —OR⁸; or pharmaceutically acceptable salt thereof.

In another embodiment of the invention A is 5- to 10-membered heteroaryl substituted with three R⁷; wherein each R⁷ is independently —(CH₂)ₜ—CF₃, cyano, halogen, C₁₋₃alkyl, C₃₋₇cycloalkyl, or —OR⁸; or pharmaceutically acceptable salt thereof.

In any of the embodiments described above, both R³ and R⁴ are C₁₋₆alkyl; or pharmaceutically acceptable salt thereof.

In any of the embodiments described above, R³ and R⁴ are both C₁₋₆alkyl, optionally substituted by fluorine, oxo, cyano, —CF₃, C₃₋₇cycloalkyl, C₆₋₁₀aryl, 5- to 10-membered heteroaryl, or 4- to 10-membered heterocycloalkyl, wherein said cycloalkyl, aryl, heterocycloalkyl, or heteroaryl substituents can be further substituted with one to three C₁₋₆alkyl, halogen, —(CH₂)ₜ—CF₃, hydroxy, oxo, or cyano; or pharmaceutically acceptable salt thereof.

In any of the embodiments described above, R³ and R⁴ are independently C₁₋₆alkyl, and either R³ or R⁴ is substituted by —CF₃; or pharmaceutically acceptable salt thereof.

In any of the embodiments described above, R³ and R⁴ are independently C₁₋₆alkyl, and both R³ and R⁴ are substituted by —CF₃; or pharmaceutically acceptable salt thereof.

In any of the embodiments described above, R³ and R⁴ are independently C₁₋₆alkyl, and either R³ or R⁴ is substituted by C₃₋₇cycloalkyl wherein said cycloalkyl is optionally substituted with one to three C₁₋₆alkyl, fluorine, —(CH₂)ₜ—CF₃, hydroxy, oxo, or cyano; or pharmaceutically acceptable salt thereof.

In any of the embodiments described above, R³ and R⁴ are independently C₁₋₆alkyl, and both R³ and R⁴ are substituted by C₃₋₇cycloalkyl wherein said cycloalkyls are optionally independently substituted with one to three C₁₋₆alkyl, fluorine, —(CH₂)ₜ—CF₃, hydroxy, oxo, or cyano; or pharmaceutically acceptable salt thereof.

In any of the embodiments described above, R³ and R⁴ are independently C₁₋₆alkyl, and either R³ or R⁴ is substituted by 5- to 10-membered heteroaryl, wherein said heteroaryl is optionally substituted with one to three C₁₋₆alkyl, halogen, —(CH₂)ₜ—CF₃, hydroxy, oxo, or cyano; or pharmaceutically acceptable salt thereof.

In any of the embodiments described above, R³ and R⁴ are independently C₁₋₆alkyl, and both R³ and R⁴ are substituted by 5- to 10-membered heteroaryl, wherein said heteroaryls are optionally independently substituted with one to three C₁₋₆alkyl, halogen, —(CH₂)ₜ—CF₃, hydroxy, oxo, or cyano; or pharmaceutically acceptable salt thereof.

In any of the embodiments described above, R³ and R⁴ are independently C₁₋₆alkyl, and R³ is substituted by C₆₋₁₀aryl and R⁴ is substituted by C₃₋₇cycloalkyl, wherein said aryl and cycloalkyl are optionally independently substituted with one to three C₁₋₆alkyl, halogen, —(CH₂)ₜ—CF₃, hydroxy, oxo, or cyano; or pharmaceutically acceptable salt thereof.

In any of the embodiments described above, R³ and R⁴ are independently C₁₋₆alkyl, and R³ is substituted by 5- to 10-membered heteroaryl and R⁴ is substituted by C₃₋₇cycloalkyl, wherein said heteroaryl or cycloalkyl are optionally independently substituted with one to three C₁₋₆alkyl, halogen, —(CH₂)ₜ—CF₃, hydroxy, oxo, or cyano; or pharmaceutically acceptable salt thereof.

In any of the embodiments described above, R³ and R⁴ together with the nitrogen to which they are bonded form a 4- to 10-membered heterocycloalkyl, optionally substituted with one to six R⁶; or pharmaceutically acceptable salt thereof. In another embodiment, R³ and R⁴ together with the nitrogen to which they are bonded form a 4- to 10-membered heterocycloalkyl and two geminal R⁶ substituents are bonded together to form a spiro-ring system with the heterocycloalkyl, and the heterocycloalkyl is substituted with zero to four additional R⁶; or pharmaceutically acceptable salt thereof.

In any of the embodiments described above, R³ and R⁴ together with the nitrogen to which they are bonded form a 5- to 10-membered heteroaryl; or pharmaceutically acceptable salt thereof.

In any of the embodiments described above, each R¹ is independently hydrogen, and m is one; or pharmaceutically acceptable salt thereof.

In another embodiment of the invention, the compound, including the pharmaceutically acceptable salts thereof, has the structure, where the substituents are defined above:

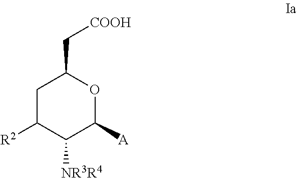

In another embodiment of the invention, the compound, including the pharmaceutically acceptable salts thereof, has the structure, where the substituents are defined above:

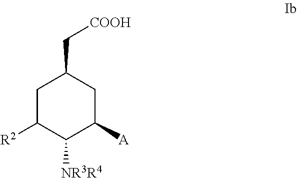

In any of the embodiments described above, R² is hydrogen.

It is understood that descriptions of any one substituent, such as R¹, may be combined with descriptions of any other substituents, such as R², such that each and every combination of the first substituent and the second substituent is provided herein the same as if each combination were specifically and individually listed. For example, in one variation, R¹ is taken together with R² to provide an embodiment wherein R¹ is methyl and R² is fluorine.

It will be understood that the compounds of formula I, and pharmaceutically acceptable salts thereof, also include hydrates, solvates and polymorphs of said compounds of formula I, and pharmaceutically acceptable salts thereof, as discussed below.

In one embodiment, the invention also relates to each of the individual compounds described as Examples 1 to 67 in the Examples section of the subject application, (including the free bases or pharmaceutically acceptable salts thereof).

In another embodiment the invention relates to a compound selected from the group consisting of:
{(1R,3S,4R)-4-(4-isopropyl-2-azaspiro[5.5]undec-2-yl)-3-[4-(trifluoromethyl)phenyl]cyclohexyl}acetic acid
{(1R,3S,4R)-4-(9-isopropyl-7-azaspiro[4.5]dec-7-yl)-3-[4-(trifluoromethyl)phenyl]cyclohexyl}acetic acid
{(1R,3S,4R)-4-(9,9-dimethyl-7-azaspiro[4.5]dec-7-yl)-3-[4-(trifluoromethyl)phenyl]cyclohexyl}acetic acid
{(1R,3S,4R)-4-(3,3,5,5-tetramethylpiperidin-1-yl)-3-[4-(trifluoromethyl)phenyl]cyclohexyl}acetic acid
{(1R,3S,4R)-4-{(cyclopentylmethyl)[(2,2-dimethylcyclopentyl)methyl]amino}-3-[4-(trifluoromethyl)phenyl]cyclohexyl}acetic acid
{(1R,3S,4R)-4-{(cyclopentylmethyl)[(3,3-dimethylcyclopentyl)methyl]amino}-3-[4-(trifluoromethyl)phenyl]cyclohexyl}acetic acid

{(1R,3S,4R)-4-[(2-chlorobenzyl)(cyclopentylmethyl)amino]-3-[4-(trifluoromethyl)phenyl]cyclohexyl}acetic acid
{(1R,3S,4R)-4-[(3-chlorobenzyl)(cyclopentylmethyl)amino]-3-[4-(trifluoromethyl)phenyl]cyclohexyl}acetic acid
{(1R,3S,4R)-4-[(4-chlorobenzyl)(cyclopentylmethyl)amino]-3-[4-(trifluoromethyl)phenyl]cyclohexyl}acetic acid

{(1R,3S,4R,5S)-4-[bis(cyclopentylmethyl)amino]-3-methyl-5-[4-(trifluoromethyl)phenyl]cyclohexyl}acetic acid;
{(1R,3R,4R,5S)-4-[bis(cyclopentylmethyl)amino]-3-methyl-5-[4-(trifluoromethyl)phenyl]cyclohexyl}acetic acid;
{(1R,3S,4R)-4-(1,2,4,5-tetrahydro-3H-1,5-methano-3-benzazepin-3-yl)-3-[4-(trifluoromethyl)phenyl]cyclohexyl}acetic acid;
2-((2S,4S,5R,6S)-5-(bis(cyclopentylmethyl)amino)-4-methyl-6-(4-(trifluoromethyl)phenyl)tetrahydro-2H-pyran-2-yl)acetic acid
{(1R,3S,4R)-4-{(cyclopentylmethyl)[(2,2-dimethylcyclohexyl)methyl]amino}-3-[4-(trifluoromethyl)phenyl]cyclohexyl}acetic acid;
{(2S,5R,6S)-5-(13-azadispiro[4.1.4.3]tetradec-13-yl)-6-[4-(trifluoromethyl)phenyl]tetrahydro-2H-pyran-2-yl}acetic acid;
{(1R,3S,4R)-4-(6-oxa-13-azadispiro[4.1.4.3]tetradec-13-yl)-3-[4-(trifluoromethyl)phenyl]cyclohexyl}acetic acid;
{(1R,3S,4R)-4-(14-azadispiro[4.2.4.3]pentadec-14-yl)-3-[4-(trifluoromethyl)phenyl]cyclohexyl}acetic acid;
{(1R,3S,4R)-4-(11-azadispiro[3.1.3.3]dodec-11-yl)-3-[4-(trifluoromethyl)phenyl]cyclohexyl}acetic acid;
{(1R,3S,4R)-4-(2,8-dioxa-11-azadispiro[3.1.3.3]dodec-11-yl)-3-[4-(trifluoromethyl)phenyl]cyclohexyl}acetic acid;
{(1R,3S,4R)-4-(2-oxa-11-azadispiro[3.1.3.3]dodec-11-yl)-3-[4-(trifluoromethyl)phenyl]cyclohexyl}acetic acid;
{(1R,3S,4R)-4-(9-azadispiro[2.1.2.3]dec-9-yl)-3-[4-(trifluoromethyl)phenyl]cyclohexyl}acetic acid;
{(1R,3S,4R)-4-(15-azadispiro[5.1.5.3]hexadec-15-yl)-3-[4-(trifluoromethyl)phenyl]cyclohexyl}acetic acid;
{(1R,3S,4R)-4-(3-oxa-15-azadispiro[5.1.5.3]hexadec-15-yl)-3-[4-(trifluoromethyl)phenyl]cyclohexyl}acetic acid;
{(1R,3S,4R)-4-(14-azadispiro[4.1.5.3]pentadec-14-yl)-3-[4-(trifluoromethyl)phenyl]cyclohexyl}acetic acid;
{(1R,3S,4R)-4-(13-azadispiro[3.1.5.3]tetradec-13-yl)-3-[4-(trifluoromethyl)phenyl]cyclohexyl}acetic acid;
{(1R,3S,4R)-4-(12-azadispiro[3.1.4.3]tridec-12-yl)-3-[4-(trifluoromethyl)phenyl]cyclohexyl}acetic acid;

and the pharmaceutically acceptable salts of each of the foregoing.

In another embodiment the present invention provides methods of treating neurological and psychiatric disorders comprising: administering to a patient in need thereof an amount of a compound of formula I effective in treating such disorders. Neurological and psychiatric disorders include but are not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering to the mammal an effective amount of a compound of formula I or pharmaceutically acceptable salt thereof. Accordingly, in one embodiment, the invention provides a method for treating a condition in a mammal, such as a human, selected from the conditions above, comprising administering a compound of formula I to the mammal. The mammal is preferably a mammal in need of such treatment. As examples, the invention provides a method for treating attention deficit/hyperactivity disorder, schizophrenia and Alzheimer's Disease.

In another embodiment the present invention provides methods of treating neurological and psychiatric disorders comprising: administering to a patient in need thereof an amount of a compound of formula I effective in treating such disorders. The compound of formula I is optionally used in combination with another active agent. Such an active agent may be, for example, an atypical antipsychotic, a cholinesterase inhibitor, Dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

The invention is also directed to a pharmaceutical composition comprising a compound of formula I, and a pharmaceutically acceptable carrier. The composition may be, for example, a composition for treating a condition selected from the group consisting of neurological and psychiatric disorders, including but not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering an effective amount of a compound of formula I or pharmaceutically acceptable salt thereof, and a pharmaceutically acceptable carrier. The composition optionally further comprises an atypical antipsychotic, a cholinesterase inhibitor, Dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

### DEFINITIONS

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, isoamyl, hexyl and the like. In some instances, the number of carbon atoms in a hydrocarbyl substituent (i.e., alkyl, alkenyl, cycloalkyl, aryl, etc.) is indicated by the prefix “C_{x-y},” wherein x is the minimum and y is the maximum number of carbon atoms in the substituent. Thus, for example, “C₁₋₆alkyl” refers to an alkyl substituent containing from 1 to 6 carbon atoms.

“Alkenyl” refers to an aliphatic hydrocarbon having at least one carbon-carbon double bond, including straight chain, branched chain or cyclic groups having at least one carbon-carbon double bond. Preferably, it is a medium size alkenyl having 2 to 6 carbon atoms. For example, as used herein, the term “C₂₋₆alkenyl” means straight or branched chain unsaturated radicals of 2 to 6 carbon atoms, including, but not limited to ethenyl, 1-propenyl, 2-propenyl (allyl), isopropenyl, 2-methyl-1-propenyl, 1-butenyl, 2-butenyl, and the like; optionally substituted by 1 to 5 suitable substituents as defined above such as fluoro, chloro, trifluoromethyl, C₁₋₆alkoxy, C₆₋₁₀aryloxy, trifluoromethoxy, difluoromethoxy or C₁₋₆alkyl. When the compounds of the invention contain a C₂₋₆alkenyl group, the compound may exist as the pure E (entgegen) form, the pure Z (zusammen) form, or any mixture thereof.

“Alkylidene” refers to a divalent group formed from an alkane by removal of two hydrogen atoms from the same carbon atom, the free valencies of which are part of a double bond.

“Alkynyl” refers to an aliphatic hydrocarbon having at least one carbon-carbon triple bond, including straight chain, branched chain or cyclic groups having at least one carbon-carbon triple bond. Preferably, it is a lower alkynyl having 2 to 6 carbon atoms. For example, as used herein, the term “C₂₋₆alkynyl” is used herein to mean a straight or branched hydrocarbon chain alkynyl radical as defined above having 2 to 6 carbon atoms and one triple bond.

The term “cycloalkyl” refers to a carbocyclic substituent obtained by removing a hydrogen from a saturated carbocyclic molecule and having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5- to 10-membered heteroaromatic ring, wherein a group having such a fused cycloalkyl group as a substituent is bound to a carbon atom of the cycloalkyl group. When such a fused cycloalkyl group is substituted with one or more substituents, the one or more substituents, unless otherwise specified, are each bound to a carbon atom of the cycloalkyl group. The fused C₆-C₁₀ aromatic ring or 5- to 10-membered heteroaromatic ring may be optionally substituted with halogen, C₁₋₆alkyl, C₃₋₁₀cycloalkyl, or ═O.

A cycloalkyl may be a single ring, which typically contains from 3 to 6 ring atoms. Examples include cyclopropyl, cyclobutyl, cyclopentyl, and cyclohexyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄₋₁₀ carbocyclic ring, such as a C₅ or a C₆ carbocyclic ring, or to a 4-to 10-membered heterocyclic ring, wherein a group having such a fused aryl group as a substituent is bound to an aromatic carbon of the aryl group. When such a fused aryl group is substituted with one or more substituents, the one or more substituents, unless otherwise specified, are each bound to an aromatic carbon of the fused aryl group. The fused C₄₋₁₀ carbocyclic or 4- to 10-membered heterocyclic ring may be optionally substituted with halogen, C₁₋₆alkyl, C₃₋₁₀cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of atoms in a cyclic substituent containing one or more heteroatoms (i.e., heteroaryl or heterocycloalkyl) is indicated by the prefix “X- to Y-membered”, wherein X is the minimum and Y is the maximum number of atoms forming the cyclic moiety of the substituent. Thus, for example, 5- to 8-membered heterocycloalkyl refers to a heterocycloalkyl containing from 5 to 8 atoms, including one or more heteroatoms, in the cyclic moiety of the heterocycloalkyl.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents are attached include, for example, alcohols, enols and phenol.

The term “cyano” (also referred to as “nitrile”) means —CN, which also may

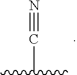

be depicted:

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is fluorine. In another embodiment, the halogen is bromine.

The term “heterocycloalkyl” refers to a substituent obtained by removing a hydrogen from a saturated or partially saturated ring structure containing a total of 4 to 14 ring atoms, wherein at least one of the ring atoms is a heteroatom selected from oxygen, nitrogen, or sulfur. For example, as used herein, the term “4- to 10-membered heterocycloalkyl” means the substituent is a single ring with 4 to 10 total members. A heterocycloalkyl alternatively may comprise 2 or 3 rings fused together, wherein at least one such ring contains a heteroatom as a ring atom (i.e., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆₋₁₀ aromatic ring or to a 5- to 10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one or more substituents, the one or more substituents, unless otherwise specified, are each bound to a heteroatom of the heterocycloalkyl group or to a carbon atom of the heterocycloalkyl group. The fused C₆₋₁₀ aromatic ring or 5- to 10-membered heteroaromatic ring may be optionally substituted with halogen, C₁₋₆alkyl, C₃₋₁₀cycloalkyl, C₁₋₆alkoxy, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include but are not limited to: 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused ring substituents such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls and heterocycloalkyls include but are not limited to furanyl, dihydrofuranyl, tetrahydrofuranyl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, isoxazolinyl, thiazolyl, isothiazolyl, thiazolinyl, isothiazolinyl, thiazolidinyl, isothiazolidinyl, thiadiazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), morpholinyl, azepinyl, oxepinyl, thiepinyl, and diazepinyl.

Examples of 2-fused-ring heteroaryls include but are not limited to indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include but are not limited to 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include but are not limited to benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄₋₁₀ carbocyclic ring, such as a C₅ or a C₆ carbocyclic ring, or to a 4- to 10-membered heterocyclic ring, wherein a group having such a fused heteroaryl group as a substituent is bound to an aromatic carbon of the heteroaryl group or to a heteroatom of the heteroaryl group. When such a fused heteroaryl group is substituted with one or more substituents, the one or more substitutents, unless otherwise specified, are each bound to an aromatic carbon of the heteroaryl group or to a heteroatom of the heteroaryl group. The fused C₄₋₁₀ carbocyclic or 4- to 10-membered heterocyclic ring may be optionally substituted with halogen, C₁₋₆alkyl, C₃₋₁₀cycloalkyl, or ═O.

Additional examples of heteroaryls and heterocycloalkyls include but are not limited to: 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzo[1,3]dioxine, benzo[1,4]dioxine, benzopyrrolidinyl, benzopiperidinyl, benzoxolanyl, benzothiolanyl, 4,5,6,7-tetrahydropyrazol[1,5-a]pyridine, benzothianyl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-2-yl (C-attached).

A substituent is “substitutable” if it comprises at least one carbon or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R″, wherein R′ and R″ together with the nitrogen atom to which they are attached may form a heterocyclic ring comprising 1 or 2 heteroatoms independently selected from oxygen, nitrogen, or sulfur, wherein said heterocycloalkyl moiety may be optionally substituted. The heterocyclic ring formed from R′ and R″ together with the nitrogen atom to which they are attached may be partially or fully saturated, or aromatic. In one embodiment, the heterocyclic ring consists of 4 to 10 atoms. In another embodiment, the heterocyclic ring is selected from the group consisting of piperidinyl, morpholinyl, azetidinyl, pyrrolyl, imidazolyl, pyrazolyl, triazolyl, and tetrazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁₋₆— prefix on C₁₋₆alkylcycloalkyl means that the alkyl moiety of the alkylcycloalkyl contains from 1 to 6 carbon atoms; the C₁₋₆— prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution only occurs on the alkyl moiety, the substituent would be described as “alkoxyhaloalkyl.” If the halogen substitution occurs on both the alkyl moiety and the alkoxy moiety, the substituent would be described as “haloalkoxyhaloalkyl.”

If substituents are described as being “independently selected” from a group, each substituent is selected independent of the other(s). Each substituent therefore may be identical to or different from the other substituent(s).

As used herein the term “Formula I” may be hereinafter referred to as a “compound(s) of the invention.” Such terms are also defined to include all forms of the compound of Formula I, including hydrates, solvates, isomers, crystalline and non-crystalline forms, isomorphs, polymorphs, and metabolites thereof. For example, the compounds of Formula I, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of Formula I may exist as clathrates or other complexes. Included within the scope of the invention are complexes such as clathrates, drug-host inclusion complexes wherein, in contrast to the aforementioned solvates, the drug and host are present in stoichiometric or non-stoichiometric amounts. Also included are complexes of Formula I containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

The compounds of Formula I may have asymmetric carbon atoms. The carbon-carbon bonds of the compounds of Formula I may be depicted herein using a solid line (—) a solid wedge ( [symbol] ) or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula I may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula I can exist as enantiomers and diastereomers or as racemates and mixtures thereof. The use of a solid line to depict bonds to one or more asymmetric carbon atoms in a compound of Formula I and the use of a solid or dotted wedge to depict bonds to other asymmetric carbon atoms in the same compound is meant to indicate that a mixture of diastereomers is present.

Stereoisomers of Formula I include cis and trans isomers, optical isomers such as R and S enantiomers, diastereomers, geometric isomers, rotational isomers, conformational isomers, and tautomers of the compounds of Formula I, including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine. It is understood that a diastereomeric mixture may form upon salt formation through protonation of the tertiary amine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of Formula I may exhibit the phenomena of tautomerism and structural isomerism. For example, the compounds of Formula I may exist in several tautomeric forms, including the enol and imine forms, and the keto and enamine forms, and geometric isomers and mixtures thereof. All such tautomeric forms are included within the scope of compounds of Formula I. Tautomers exist as mixtures of a tautomeric set in solution. In solid form, usually one tautomer predominates. Even though one tautomer may be described, the present invention includes all tautomers of the compounds of Formula I.

The present invention also includes isotopically-labeled compounds, which are identical to those recited in Formula I above, but for the fact that one or more atoms are replaced by an atom of the same atomic number, but having an atomic mass or mass number different from the predominant atomic mass or mass number usually found in nature. Examples of isotopes that may be incorporated into compounds of Formula I include isotopes of hydrogen, carbon, nitrogen, oxygen, phosphorus, fluorine and chlorine, such as, but not limited to, ²H, ³H, ¹³C, ¹⁴C, ¹⁵N, ¹⁸O, ¹⁷O, ³²P, ³⁵S, ¹⁸F, and ³⁶Cl. Certain isotopically-labeled compounds of Formula I, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of Formula I may generally be prepared by carrying out the procedures disclosed in the Schemes and/or in the Examples and Preparations below, by substituting an isotopically-labeled reagent for a non-isotopically-labeled reagent.

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention, which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include but are not limited to aliphatic, cycloaliphatic, aromatic, araliphatic, heterocyclic, carboxylic, and sulfonic classes of organic acids.

Specific examples of suitable organic acids include but are not limited to acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, and undecanoate.

Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diethanolamine, glycine, lysine, meglumine, olamine, tromethamine and zinc salts.

Organic salts may be made from secondary, tertiary or quaternary amine salts, such as tromethamine, diethylamine, N,N-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (i.e., dimethyl, diethyl, dibutyl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

Typically, a compound of the invention is administered in an amount effective to treat a condition as described herein. The compounds of the invention are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts. The term “therapeutically effective amount” as used herein refers to that amount of the compound being administered which will relieve to some extent one or more of the symptoms of the disorder being treated.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, the total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the invention comprises the use of one or more compounds of the invention for the preparation of a medicament for the treatment of the conditions recited herein.

For the treatment of the conditions referred to above, the compounds of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formula I are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (i.e., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas.

When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88 (10), 955-958 (1999).

Formulations suitable for topical administration to the eye include, for example, eye drops wherein the compound of this invention is dissolved or suspended in a suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed-linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone; as a mixture, for example, in a dry blend with lactose; or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

The compounds of the present invention can be used, alone or in combination with other therapeutic agents, in the treatment of various conditions or disease states. The compound(s) of the present invention and other therapeutic agent(s) may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

In another embodiment, the invention relates to the novel intermediates useful for preparing the compounds of the invention.

### General Synthetic Schemes

The compounds of formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-XII (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991; and T. W. Greene and P. G. M. Wuts, Protective Groups in Organic Chemistry, John Wiley & Sons, 1999, which are hereby incorporated by reference.

Compounds of formula I, or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

### General Schemes

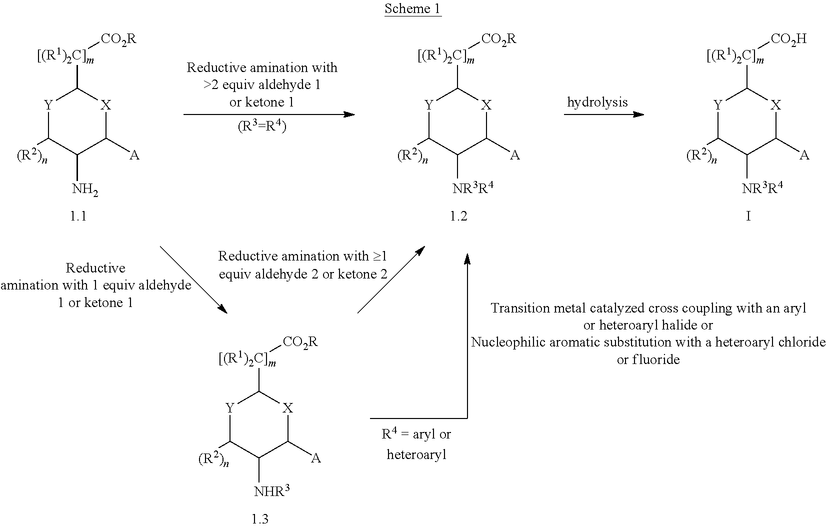

Scheme 1 illustrates a method for the preparation of compounds depicted by formula I. This method commences with reductive amination of the primary amine of formula 1.1 to provide tertiary amine 1.2 using one of several methods known to those skilled in the art. For example, the compound of formula 1.1 may be treated with an excess of aldehyde 1 or ketone 1 and a suitable reducing agent such as sodium triacetoxyborohydride to furnish 1.2 where R³═R⁴. Alternatively, the compound of formula 1.2 (where R³≈R⁴) may be prepared by two sequential reductive aminations, first with aldehyde 1 or ketone 1 to give the compound of formula 1.3 followed by aldehyde 2 or ketone 2 to give the compound of formula 1.2. The compound of the formula 1.2, where R⁴ is aryl or heteroaryl, can be prepared by transition metal-catalyzed cross coupling of the amine of the formula 1.3 with an appropriate aryl or heteroaryl halide using methods known to those skilled in the art such as Buchwald-Hartwig conditions. Alternatively, a nucleophilic aromatic substitution reaction between the amine of formula 1.3 and an appropriate heteroaryl halide can be employed to furnish the compound of the formula 1.2. The resulting ester of formula 1.2 is then hydrolyzed by treating with aqueous base such as KOH, LiOH, or NaOH in a solvent such as MeOH or THF or a mixture thereof to give compounds of formula I.

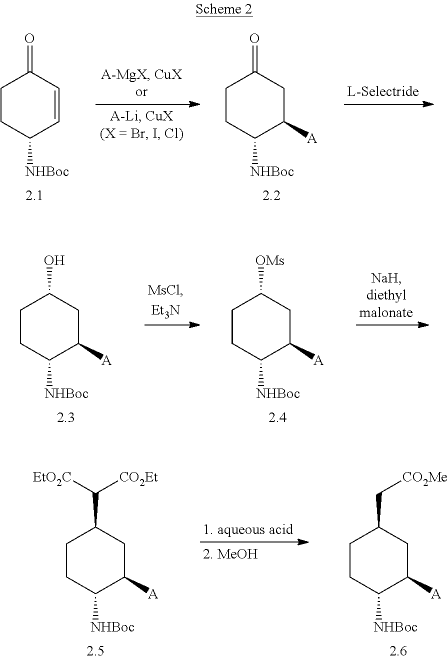

Scheme 2 illustrates a method for the preparation of intermediates of formula 2.6. The enone of formula 2.1 is subjected to 1,4-addition of a cuprate derived from a suitable organometallic species such as a Grignard reagent and a copper(I) source such as CuBr-DMS in a solvent such as THF. The resulting ketone of formula 2.2 is treated with a reducing agent such as L-Selectride to furnish the alcohol of formula 2.3. Exposure of a compound of formula 2.3 to mesyl chloride in the presence of an amine base such as triethylamine provides the mesylate of formula 2.4, which may be converted to the intermediate of formula 2.5 by heating in the presence of diethyl malonate and sodium hydride in a suitable solvent such as toluene or 1,2-dimethoxyethane. The diester of formula 2.5 is then subjected to hydrolysis, decarboxylation, and Fischer esterification by heating in the presence of an aqueous acid such as 6 N HCl followed by addition of methanol to provide the target intermediates of formula 2.6.

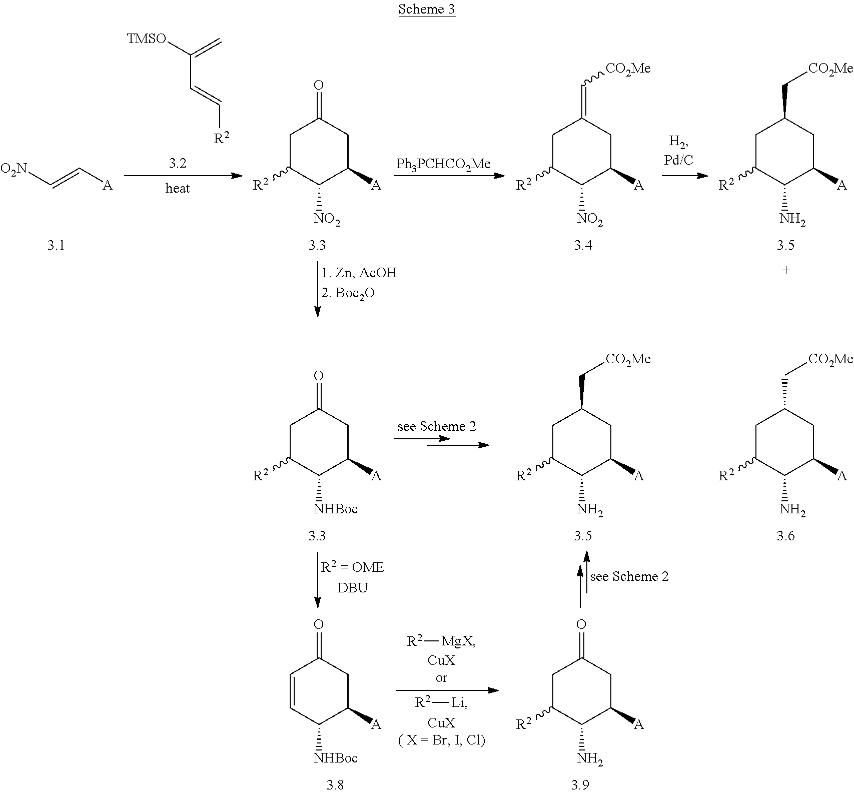

Scheme 3 illustrates a method for preparing compounds depicted by formulas 3.5 and 3.6. This method commences with heating β-nitrostyrene dienophiles of formula 3.1 with dienes of formula 3.2 (i.e., 2-trimethylsilyloxy-1,3-butadienes) to afford nitrocyclohexanones of formula 3.3 (J. Am. Chem. Soc. 1953, 75, 1912). Wittig olefination of ketones of formula 3.3 with reagents such as methyl (triphenylphosphoranylidene)acetate provides intermediates of formula 3.4 as a separable mixture of E and Z isomers. The olefin and nitro group can be reduced under a variety of conditions including hydrogenation using Pd/C as a catalyst to give a diastereomeric mixture of compounds of formulas 3.5 and 3.6. Alternatively, a stepwise process involving reduction of the nitro group under a variety of conditions including zinc in acetic acid, followed by hydrogenation of the alkene over catalysts such as Rh provide compounds of formula 3.5 as the major diastereomer (˜3/1). Scheme 3 also describes two additional methods for the preparation of compounds of formula 3.5. Reduction of the nitro group in formula 3.3 under a variety of conditions including zinc and acetic acid, followed by Boc protection of the resulting amine provides compounds of formula 3.7. This intermediate can be converted to compounds of formula 3.5 in a similar fashion as shown in Scheme 2 for the conversion of 2.2 to 2.6. Alternatively, compounds of formula 3.7 where R² is methoxy can be treated with a base such as DBU to give compounds of formula 3.8. Various R² groups can be introduced onto compounds of formula 3.8 by 1,4-conjugate addition of a cuprate derived from a suitable organometallic species such as a Grignard reagent and a copper(I) source such as CuBr-DMS, to give compounds of the formula 3.9. These can be converted to compounds of formula 3.5 using a similar method to that shown in Scheme 2 for the conversion of 2.2 to 2.6.

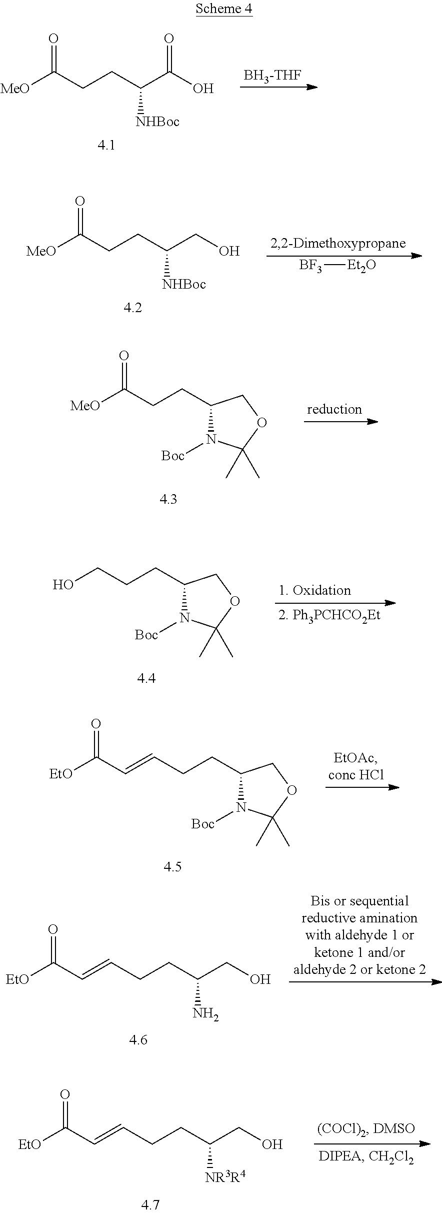

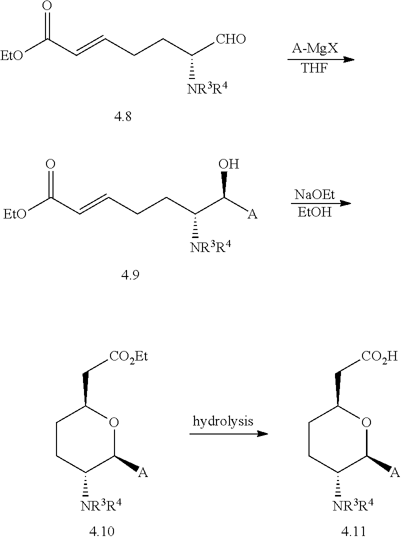

Scheme 4 illustrates a method for preparing compounds depicted by formula 4.11. This method involves the reduction of the acid moiety of N-tert-butoxycarbonyl D-glutamic acid ester 4.1 by treatment with borane or other suitable reducing agents. The primary alcohol of formula 4.2 is reacted with 2,2-dimethoxypropane in the presence of BF₃—OEt₂ or p-toluenesulfonic acid to afford the dimethyloxazolidine of formula 4.3. The ester moiety of formula 4.3 is reduced using a suitable reducing agent such as LiAlH₄ to provide the primary alcohol of formula 4.4. The alcohol of formula 4.4 can be oxidized under a variety of conditions known to those skilled in the art, for instance via Swern oxidation, to give the intermediate aldehyde, which is converted to the α,β-unsaturated ester of formula 4.5 under olefination conditions such as treatment with ethyl (triphenylphosphoranylidene)acetate. Treatment of a compound of formula 4.5 with concentrated HCl provides the aminoalcohol of formula 4.6. The aminoalcohol of formula 4.6 may be treated with an excess of aldehyde 1 or ketone 1 and a suitable reducing agent such as sodium triacetoxyborohydride to provide the substituted amine of formula 4.7 (R³═R⁴). Alternatively, compounds of formula 4.6 are treated sequentially with aldehyde 1 or ketone 1 and a reducing agent such as Na(OAc)₃BH followed by aldehyde 2 or ketone 2 and a reducing agent such as Na(OAc)₃BH to provide compounds of formula 4.7 (R³≈R⁴). Swern oxidation of compounds of formula 4.7 under conditions that avoid epimerization, such as using diisopropylethylamine as base, provides aldehydes of formula 4.8, which can be treated with a Grignard reagent to provide alcohols of formula 4.9 (Angew. Chem., Int. Ed. Engl. 1991, 30, 1531). Alternatively, the oxidation of alcohols of formula 4.7 can be accomplished using other methods known to those skilled in the art, such as Parikh-Doering conditions (J. Am. Chem. Soc. 1967, 89, 5505), to provide aldehydes of formula 4.8. Cyclization of compounds of formula 4.9 can be accomplished by treatment with sodium ethoxide, potassium tert-butoxide, tetrabutylammonium fluoride or other suitable bases to provide tetrahydropyrans of formula 4.10. The ester function of a compound of formula 4.10 is then hydrolyzed to provide carboxylic acids of formula 4.11 by treating with aqueous base such as KOH, LiOH or NaOH in a solvent such as MeOH or THF or combination thereof.

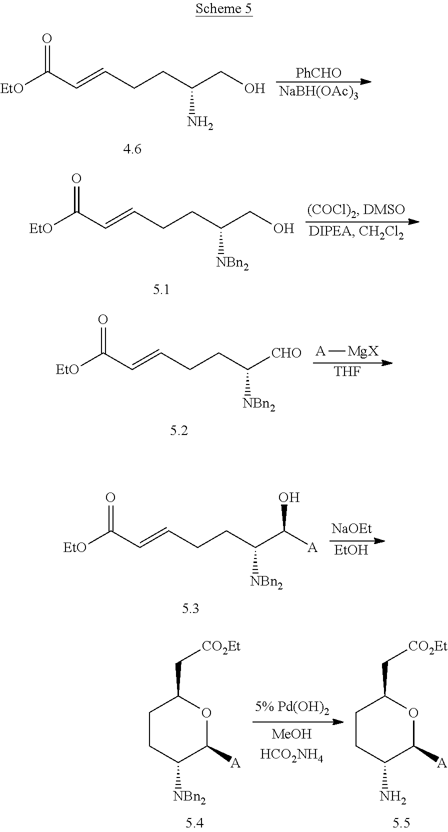

Scheme 5 illustrates a method for preparing compounds depicted by formula 5.5. The aminoalcohol of formula 4.6 is treated with benzaldehyde and NaBH(OAc)₃ to provide the bisbenzyl amine of formula 5.1. Swern oxidation of a compound of formula 5.1 under conditions that avoid epimerization, such as using diisopropylethylamine as base, provides the aldehyde of formula 5.2 which can be treated with aryl Grignard reagents to provide alcohols of formula 5.3 (Angew. Chem., Int. Ed. Engl. 1991, 30, 1531). Cyclization of compounds of formula 5.3 can be effected with sodium ethoxide, potassium tert-butoxide, tetrabutylammonium fluoride or other suitable bases to provide tetrahydropyrans of formula 5.4. Hydrogenolysis of a compound of formula 5.4 using methods known to those skilled in the art such as ammonium formate in the presence of Pd(OH)₂ or other suitable catalyst provides compounds of formula 5.5.

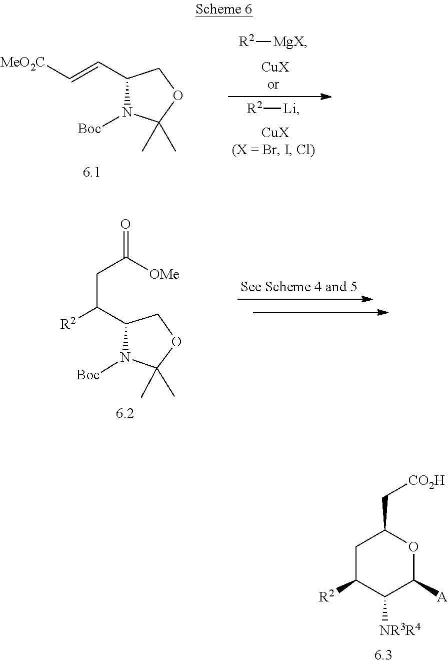

Scheme 6 illustrates a method for preparing compounds depicted by formula 6.3. Various R² groups can be introduced by 1,4-conjugate addition of a cuprate derived from a suitable organometallic species such as a Grignard reagent and a copper(I) source such as CuBr-DMS to compounds of formula 6.1, to give compounds of formula 6.2. Compounds of formula 6.2 can be converted to compounds of the formula 6.3 using a similar method to that shown in Scheme 4 for the conversion of 4.3 to 4.11.

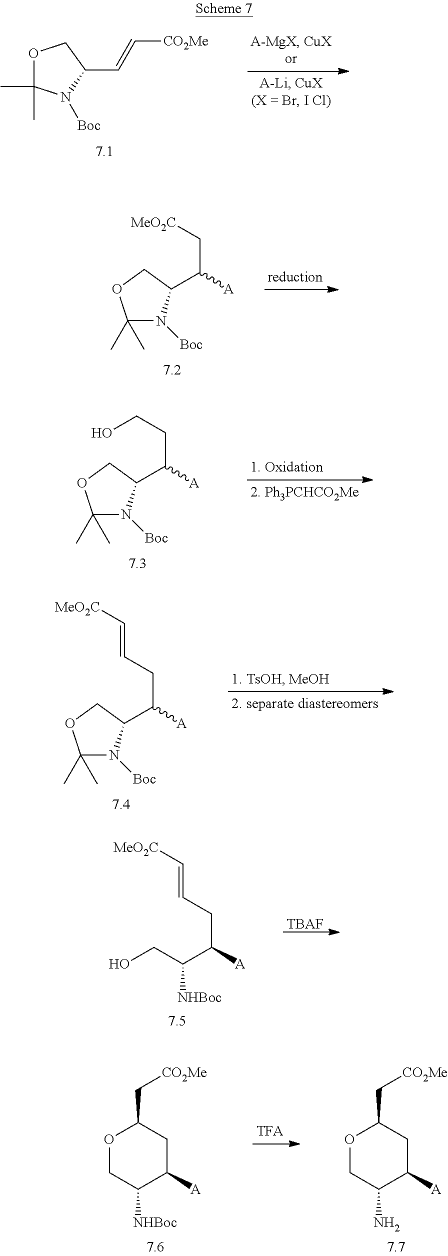

Scheme 7 illustrates a method for preparing compounds depicted by formula 7.7. The α,β0-unsaturated ester of formula 7.1 is subjected to 1,4-conjugate addition of a cuprate derived from a suitable organometallic species such as an aryl Grignard reagent and a copper(I) source such as CuBr-DMS, optionally including an additive such as trimethylsilyl chloride, to provide compounds of formula 7.2. The ester compound of formula 7.2 is treated with a suitable reducing agent such as LiAlH₄ to provide the primary alcohol of formula 7.3. The alcohol of formula 7.3 is oxidized under a variety of conditions known to those skilled in the art including Swern oxidation to give the intermediate aldehyde, which is converted to the α,β-unsaturated ester of formula 7.4 under olefination conditions such as treatment with methyl (triphenylphosphoranylidene)acetate. The acetonide moiety of formula 7.4 is selectively deprotected by treatment with an acid such as p-toluenesulfonic acid in a solvent such as MeOH and the minor diastereomer is separated to give the aminoalcohol of formula 7.5. Cyclization of compounds of formula 7.5 can be accomplished by treatment with sodium methoxide, potassium tert-butoxide, tetrabutylammonium fluoride or other suitable bases to provide tetrahydropyrans of formula 7.6. The Boc group can be removed using conditions known to those skilled in the art such as trifluoroacetic acid to give compounds of formula 7.7.

### EXPERIMENTAL PROCEDURES AND WORKING EXAMPLES

The following illustrate the synthesis of various compounds of the present invention. Additional compounds within the scope of this invention may be prepared using the methods illustrated in these Examples, either alone or in combination with techniques generally known in the art.

It will be understood that the intermediate compounds of the invention depicted below are not limited to the particular enantiomer shown, but also include all stereoisomers and mixtures thereof. It will also be understood that compounds of Formula I can include intermediates of compounds of Formula I.

### Experimental Procedures

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Mass spectrometry data is reported from either liquid chromatography-mass spectrometry (LCMS) or atmospheric pressure chemical ionization (APCI). Chemical shifts for nuclear magnetic resonance (NMR) data are expressed in parts per million (ppm, δ) referenced to residual peaks from the deuterated solvents employed.

For syntheses referencing procedures in other Examples, Preparations or Methods, reaction conditions (length of reaction and temperature) may vary. In general, reactions were followed by thin layer chromatography or mass spectrometry, and subjected to work-up when appropriate. Purifications may vary between experiments: in general, solvents and the solvent ratios used for eluants/gradients were chosen to provide appropriate R_{f}s or retention times.

### Preparations

### Preparation 1

### Methyl {(1R,3S,4R)-4-amino-3-[4-(trifluoromethyl)phenyl]cyclohexyl}acetate (P1)

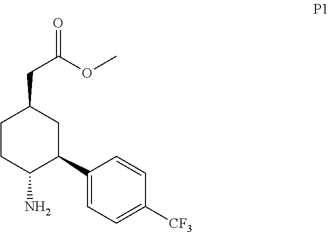

### Step 1. Synthesis of tert-butyl {(1R,2S)-4-oxo-2-[4-(trifluoromethyl)phenyl]cyclohexyl}carbamate

To a cooled (−78° C.) suspension of CuBr-dimethyl sulfide (12.3 g, 59.2 mmol) in THF (50 mL) was added 4-(trifluoromethyl)phenylmagnesium bromide (0.7 M in THF, 169 mL, 118 mmol) drop-wise over 30 minutes. The resulting mixture was stirred at −78° C. for 1 hour. A solution of tert-butyl [(1R)-4-oxocyclohex-2-en-1-yl]carbamate (J. Chem. Soc., Perkin Trans. 1 2000, 329-343) (5.0 g, 20 mmol) in THF (50 mL) was then added drop-wise over 10 minutes. Upon completion of the addition, the reaction was quenched with saturated aqueous NH₄Cl solution (125 mL) and allowed to warm to room temperature. The mixture was extracted with EtOAc and the combined organic layers were washed with saturated aqueous NaCl solution and dried over MgSO₄. The solvent was removed under reduced pressure and the residue was purified by silica gel chromatography (Gradient: 5% to 50% EtOAc in heptane) to provide the title compound as a white solid (6 g, 70%).

### Step 2. Synthesis of tert-butyl {(1R,2S,4S)-4-hydroxy-2-[4-(trifluoromethyl)phenyl]cyclohexyl}carbamate

tert-Butyl {(1R,2S)-4-oxo-2-[4-(trifluoromethyl)phenyl]cyclohexyl}carbamate (6.0 g, 16.8 mmol) was dissolved in THF (168 mL) and cooled to −78° C. To this solution was added L-Selectride (1.0 M in THF, 37 mL, 37 mmol) drop-wise. The mixture was then allowed to warm slowly to room temperature over 18 hours. The reaction was quenched with saturated aqueous NH₄Cl solution (125 mL) and the mixture extracted with EtOAc. The combined organic layers were washed with saturated aqueous NaCl solution and dried over MgSO₄. After filtration, the solvent was removed under reduced pressure and the residue was purified by silica gel chromatography (Gradient: 0% to 50% EtOAc in heptane) to provide the title compound (4.5 g, 75%). LCMS m/z 304.4 ([M−2-methylprop-1-ene]+1).

### Step 3. Synthesis of (1S,3S,4R)-4-[(tert-butoxycarbonyl)amino]-1-3-[4-(trifluoromethyl)phenyl]cyclohexyl methanesulfonate

tert-Butyl {(1R,2S,4S)-4-hydroxy-2-[4-(trifluoromethyl)phenyl]cyclohexyl}carbamate (4.5 g, 12.5 mmol) and triethylamine (2.27 mL, 16.3 mmol) were combined in CH₂Cl₂ (79 mL) and cooled to 0° C. Methanesulfonyl chloride (1.20 mL, 15 mmol) was added and the mixture was stirred for 30 minutes at 0° C. then warmed to room temperature. After 1 hour, the mixture was washed with saturated aqueous NaHCO₃ solution and water. The organic layer was dried over MgSO₄, filtered and the filtrate concentrated to provide the title compound, which was used without further purification (5.48 g, quant.).

### Step 4. Synthesis of diethyl {(1R,3S,4R)-4-[(tert-butoxycarbonyl)amino]-3-[4-(trifluoromethyl)phenyl]cyclohexyl}malonate

To a suspension of NaH (544 mg, 22.7 mmol) in 1,2-dimethoxyethane (30 mL) was added diethyl malonate (3.87 mL, 25.5 mmol) and the mixture was stirred at room temperature for 2 hours. To this mixture was added a solution of (1S,3S,4R)-4-[(tert-butoxycarbonyl)amino]-3-[4-(trifluoromethyl)phenyl]cyclohexyl methanesulfonate (3.72 g, 8.5 mmol) in 1,2-dimethoxyethane (10 mL). The resulting solution was heated to reflux. After 36 hours, the reaction was cooled to room temperature and quenched with saturated aqueous NH₄Cl solution (50 mL). The mixture was taken up in EtOAc and washed with H₂O and saturated aqueous NaCl solution. The organic layer was dried over MgSO₄, filtered and the solvent was removed under reduced pressure. The residue was precipitated from CH₂Cl₂/heptane to provide the title compound as a white solid (2.95 g, 69%). LCMS m/z 446.6 ([M−2-methylprop-1-ene]+1).

### Step 5. Synthesis of methyl {(1R,3S,4R)-4-amino-3-[4-(trifluoromethyl)phenyl]cyclohexyl}acetate (P1)

Diethyl {(1R,3S,4R)-4-[(tert-butoxycarbonyl)amino]-3-[4-(trifluoromethyl)phenyl]cyclohexyl}malonate (2.88 g, 5.74 mmol) was treated with 6 N aqueous HCl (50 mL) and the mixture was heated to reflux for 72 hours. The reaction was cooled to room temperature and solvent was removed under reduced pressure. The residue was dissolved in MeOH (50 mL), treated with a few drops of concentrated H₂SO₄ and heated to reflux. After 1 hour, the reaction was cooled to room temperature and the solvent removed under reduced pressure. The residue was dissolved in CH₂Cl₂ and washed with saturated aqueous NaHCO₃ solution. The organic layer was dried over MgSO₄, filtered and concentrated to a grayish solid which was purified by silica gel chromatography (Gradient: 0% to 20% MeOH in CH₂Cl₂) to give the title compound as an off-white solid (1.8 g, 99%). ¹H NMR (400 MHz, CDCl₃) δ 1.19-1.44 (m, 3H), 1.83-1.93 (m, 2H), 1.94-2.06 (m, 2H), 2.23 (dd, half of ABX pattern, J=15.0, 7.2 Hz, 1H), 2.29 (dd, half of ABX pattern, J=15.1, 6.9 Hz, 1H), 2.39-2.47 (m, 1H), 2.88 (ddd, J=10.5, 10.5, 3.9 Hz, 1H), 3.66 (s, 3H), 7.35 (d, J=8.1 Hz, 2H), 7.58 (d, J=8.2 Hz, 2H).

### Preparation 2

### Ethyl (2E,6R)-6-[bis(cyclopentylmethyl)amino]-7-oxohept-2-enoate (P2)

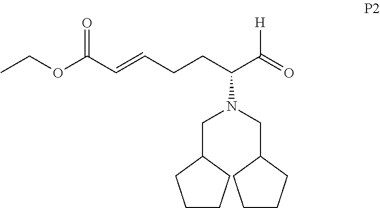

### Step 1. Synthesis of methyl (4R)-4-[(tert-butoxycarbonyl)amino]-5-hydroxypentanoate

Borane in THF (1 N, 370 mL, 0.370 mol) was cooled in an ice-salt bath to −5/−10° C. A solution of N-tert-butoxycarbonyl-D-glutamic acid γ-methyl ester (44.93 g, 0.172 mole) in THF (150 mL) was added drop-wise over 1.5 hours while maintaining the temperature below 0° C. After completion of the addition, the reaction mixture was allowed to stir at 0° C. for 2 hours, then was carefully quenched with AcOH (10% in MeOH, 75 mL). When excess borane had been decomposed, the volatiles were removed in vacuo, and the residue was partitioned between tert-butyl methyl ether and 0.5 N aqueous HCl. The organic phase was washed with saturated aqueous NaHCO₃ solution, saturated aqueous NaCl solution, dried over Na₂SO₄, and filtered. Evaporation of the solvent yielded the title compound as an oily residue (25.16 g, 59%). The material was used in the next step without further purification.

### Step 2. Synthesis of tert-butyl (4R)-4-(3-methoxy-3-oxopropyl)-2,2-dimethyl-1,3-oxazolidine-3-carboxylate

Methyl (4R)-4-[(tert-butoxycarbonyl)amino]-5-hydroxypentanoate (25.00 g, 0.101 mole) and 2,2-dimethoxypropane (105.26 g, 1.012 mole) were dissolved in acetone (300 mL) under a nitrogen atmosphere. BF₃.Et₂O (2.11 g, 0.015 mole) was added drop-wise over 15 minutes and the yellow solution was allowed to stir at room temperature for 3 hours. Triethylamine (25 mL, 0.180 mole) was added and the mixture was concentrated in vacuo. The residue was partitioned between EtOAc (800 mL) and saturated aqueous NaHCO₃ solution (150 mL). The organic phase was washed with saturated aqueous NaCl solution (100 mL), dried over Na₂SO₄, filtered, and evaporated to dryness to yield the title compound as an oily residue (30.46 g, 100%) that was used in the next step without further purification.

### Step 3. Synthesis of tert-butyl (4R)-4-(3-hydroxypropyl)-2,2-dimethyl-1,3-oxazolidine-3-carboxylate

In a 3-necked flask, LiAlH₄ (5.77 g, 0.152 mole) was suspended in THF (250 mL) at 0° C. under a nitrogen atmosphere. To this suspension was added drop-wise a solution of tert-butyl (4R)-4-(3-methoxy-3-oxopropyl)-2,2-dimethyl-1,3-oxazolidine-3-carboxylate (30.46 g, 0.101 mole) in THF (250 mL) over 1 hour, maintaining an internal temperature of 0° C. The mixture was allowed to warm to room temperature for 1 hour. When TLC indicated complete conversion of the starting material, the mixture was cooled to 0° C. The reaction was quenched by the sequential addition of water (6 mL), aqueous NaOH (4 N, 6 mL), and water (18 mL). The suspension was diluted with tert-butyl methyl ether (500 mL) and stirred at room temperature for 1 hour. The salts were removed by filtration through Celite, and the solution was concentrated in vacuo to yield the title compound as an oily residue (25.46 g, 97%), which was used in the next step without further purification. ¹H NMR (400 MHz, CDCl₃) δ 1.45 (s, 9H), 1.49-1.66 (m, 6H), 1.70 (br s, 1H), 1.81 (br s, 1H), 2.07 (br s, 1H), 3.57-3.74 (m, 3H), 3.78 (br s, 1H), 3.87-3.99 (m, 2H).

### Step 4. Synthesis of tert-butyl (4R)-4-[(3E)-5-ethoxy-5-oxopent-3-en-1-yl]-2,2-dimethyl-1,3-oxazolidine-3-carboxylate

In a 3-necked flask with dropping funnel, oxalyl chloride (17.09 g, 0.135 mole) was dissolved in CH₂Cl₂ (225 mL) and cooled to −78° C. under a nitrogen atmosphere. To this solution was added drop-wise a solution of dimethyl sulfoxide (23.21 g, 0.298 mole) in CH₂Cl₂ (115 mL) over 25 minutes, maintaining an internal temperature of <−70° C. After 30 minutes, a solution of tert-butyl (4R)-4-(3-hydroxypropyl)-2,2-dimethyl-1,3-oxazolidine-3-carboxylate (25.40 g, 0.098 mole) in CH₂Cl₂ (115 mL) was added over 40 minutes, maintaining an internal temperature of <−70° C. The mixture was stirred at −78° C. for 1 hour before triethylamine (49.65 g, 0.492 mole) was added as a solution in CH₂Cl₂ (115 mL) over 25 minutes. TLC after 20 minutes indicated complete conversion of the alcohol into the aldehyde. Ethyl (triphenylphosphoranylidene)acetate (51.27 g, 0.147 mole) was added portion-wise to the reaction mixture at −78° C. over 5 minutes. The cooling bath was removed, and after 3 hours, the orange suspension was quenched with water (400 mL), and the aqueous layer was extracted with CH₂Cl₂ (300 mL). The combined organic layers were washed with citric acid (10% solution in water, 250 mL), water (200 mL), and saturated aqueous NaCl solution (200 mL) and dried over Na₂SO₄ Filtration, evaporation of solvents and purification by silica gel chromatography (Gradient: 0% to 25% EtOAc in heptane) yielded the title compound as a thick oil (9.50 g, 30%). ¹H NMR (400 MHz, CDCl₃) δ 1.25 (t, J=7.13 Hz, 3H), 1.45 (br s, 9H), 1.54 (d, J=17.8 Hz, 3H), 1.61-1.84 (m, 2H), 1.84-2.00 (m, 1H), 2.06-2.31 (m, 2H), 3.70 (d, J=7.6 Hz, 1H), 3.78 (br s, 1H), 3.85-3.97 (m, 2H), 4.15 (q, J=7.2 Hz, 2H), 5.82 (dt, J=15.6, 1.5 Hz, 1H), 6.85-7.00 (m, 1H).

### Step 5. Synthesis of ethyl (2E,6R)-6-amino-7-hydroxyhept-2-enoate

tert-Butyl (4R)-4-[(3E)-5-ethoxy-5-oxopent-3-en-1-yl]-2,2-dimethyl-1,3-oxazolidine-3-carboxylate (16.8 g, 51.3 mmol) was dissolved in EtOAc (100 mL) and treated with concentrated aqueous HCl (15 mL, 190 mmol). The mixture was stirred at room temperature for 80 minutes. The solvent was removed under reduced pressure and the residue was azeotroped with heptane (5×100 mL) to provide the title compound as a thick oil (9.6 g, quant). LCMS m/z 188.3 (M+1). ¹H NMR (400 MHz, CDCl₃) δ 1.24 (t, J=7.0 Hz, 3H), 1.82 (br s, 1H), 1.97 (br s, 1H), 2.38 (br s, 2H), 3.46 (br s, 1H), 3.73 (br s, 1H), 3.90 (br s, 1H), 4.13 (q, J=6.4 Hz, 2H), 5.23 (br s, 1H), 5.89 (d, J=15.4 Hz, 1H), 6.89 (m, 1H), 7.97 (br s, 2H).

### Step 6. Synthesis of ethyl (2E,6R)-6-[bis(cyclopentylmethyl)amino]-7-hydroxyhept-2-enoate

Ethyl (2E,6R)-6-amino-7-hydroxyhept-2-enoate (9.6 g, 51 mmol) and cyclopentanecarbaldehyde (14.1 mL, 128 mmol) were combined in CH₂Cl₂ (500 mL) and cooled in a water bath. Sodium triacetoxyborohydride (35 g, 160 mmol) was added in 5 g portions. The mixture was stirred at room temperature for 1 hour and then quenched with saturated aqueous NaHCO₃ solution. The organic layer was separated and the aqueous layer extracted with CH₂Cl₂. The combined organic layers were dried over MgSO₄, filtered and concentrated under reduced pressure. The residue was purified by silica gel chromatography (Gradient: 5% to 25% EtOAc in heptane) to provide the title compound as a thick oil (14.7 g, 82%). APCI m/z 352.1 (M+1). ¹H NMR (400 MHz, CDCl₃) δ 1.03-1.29 (m, 4H), 1.29 (t, J=7.1 Hz, 3H), 1.49-1.65 (m, 8H), 1.67-1.84 (m, 5H), 1.95-2.07 (m, 2H), 2.08-2.22 (m, 2H), 2.26 (dd, J=12.6, 10.1 Hz, 2H), 2.39 (dd, J=12.7, 4.8 Hz, 2H), 2.76-2.84 (m, 1H), 3.25 (t, J=10.4 Hz, 1H), 3.45-3.54 (m, 3H), 4.19 (q, J=7.1 Hz, 2H), 5.84 (dt, J=15.6, 1.5 Hz, 1H), 6.93 (dt, J=15.6, 6.8 Hz, 1H).

### Step 7. Synthesis of ethyl (2E,6R)-6-[bis(cyclopentylmethyl)amino]-7-oxohept-2-enoate (P2)

To a cooled, −78° C., solution of oxalyl chloride (9.23 mL, 106 mmol) in CH₂Cl₂ (100 mL) was added a solution of dimethyl sulfoxide (15.2 mL, 212 mmol) in CH₂Cl₂ (50 mL) over 15 minutes. After 5 minutes, a solution of ethyl (2E,6R)-6-[bis(cyclopentylmethyl)amino]-7-hydroxyhept-2-enoate (18.6 g, 52.9 mmol) in CH₂Cl₂ (100 mL) was added drop-wise over 1 hour. The mixture was stirred for an additional 10 minutes and then a solution of diisopropylethylamine (46.1 mL, 265 mmol) in CH₂Cl₂ (100 mL) was added drop-wise over 30 minutes. The resulting mixture was stirred for 30 minutes at −78° C. and then warmed to room temperature. The reaction was poured into water (150 mL) and diluted with CH₂Cl₂ (200 mL). The organic layer was separated, washed with water and saturated aqueous NaCl solution and dried over MgSO₄. After filtration, the solvent was removed under reduced pressure to provide the title compound as a thick oil, which was used without purification or characterization (18.5 g, quant.). The reaction was repeated, and NMR data was obtained on the sample. ¹H NMR (400 MHz, CDCl₃) δ 1.10-1.23 (m, 4H), 1.30 (t, J=7.1 Hz, 3H), 1.49-1.64 (m, 9H), 1.67-1.78 (m, 4H), 1.80-1.90 (m, 1H), 1.95-2.06 (m, 2H), 2.21-2.38 (m, 2H), 2.44 (d, J=7.6 Hz, 4H), 3.18 (t, J=6.6 Hz, 1H), 4.20 (q, J=7.1 Hz, 2H), 5.85 (dt, J=15.6, 1.5 Hz, 1H), 6.96 (dt, J=15.5, 6.8 Hz, 1H), 9.76 (s, 1H).

### Preparation 3 and Preparation 4

### Ethyl {(2S,5R,6S)-5-[bis(cyclopentylmethyl)amino]-6-[4-(trifluoromethyl)phenyl]tetrahydro-2H-pyran-2-yl}acetate (P3) and Ethyl {(2R,5R,6S)-5-[bis(cyclopentylmethyl)amino]-6-[4-(trifluoromethyl)phenyl]tetrahydro-2H-pyran-2-yl}acetate (P4)

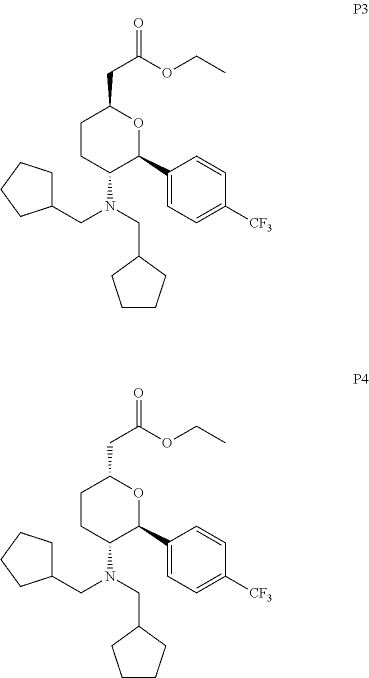

### Step 1. Synthesis of ethyl (2E,6R,7S)-6-[bis(cyclopentylmethyl)amino]-7-hydroxy-7-[4-(trifluoromethyl)phenyl]hept-2-enoate

To a cooled, −78° C., solution of 4-(trifluoromethyl)phenylmagnesium bromide in THF (200 mL, 0.66 M, 132 mmol) was added a solution of ethyl (2E,6R)-6-[bis(cyclopentylmethyl)amino]-7-oxohept-2-enoate (P2) (18.5 g, 52 mmol) in THF (125 mL) drop-wise over 45 minutes. The reaction was stirred at −78° C. for 10 minutes and then quenched with saturated aqueous NH₄Cl solution (125 mL). The mixture was warmed to room temperature and partitioned between water and tert-butyl methyl ether. The aqueous layer was extracted with tert-butyl methyl ether and the combined organic layers were washed with 1 M aqueous NaOH. The organic layer was dried over MgSO₄, filtered, and the solvent removed under reduced pressure. The residue was purified by silica gel chromatography (Gradient: 0% to 10% EtOAc in heptane) to provide the title compound as a thick oil (20.4 g, 77%). APCI m/z 496.2 (M+1). ¹H NMR (400 MHz, CDCl₃) δ 0.99-1.18 (m, 5H), 1.25 (t, J=7.1 Hz, 3H), 1.40-1.59 (m, 9H), 1.67 (d, J=17.6 Hz, 3H), 1.86-2.04 (m, 2H), 2.04-2.22 (m, 2H), 2.30 (dd, J=13.0, 6.6 Hz, 2H), 2.33-2.39 (m, 1H), 2.43 (dd, 2H), 2.69 (d, J=5.2 Hz, 1H), 2.83 (dt, J=8.2, 4.3 Hz, 1H), 4.14 (q, J=7.1 Hz, 2H), 4.97 (t, J=4.0 Hz, 1H), 5.72 (dt, J=15.7, 1.6 Hz, 1H), 6.88 (dt, J=15.7, 6.7 Hz, 1H), 7.40 (d, J=8.0 Hz, 2H). 7.57 (d, J=8.3 Hz, 2H).

### Step 2. Synthesis of ethyl {(2S,5R,6S)-5-[bis(cyclopentylmethyl)amino]-6-[4-(trifluoromethyl)phenyl]tetrahydro-2H-pyran-2-yl}acetate (P3) and ethyl {(2R,5R,6S)-5-[bis(cyclopentylmethyl)amino]-6-[4-(trifluoromethyl)phenyl]tetrahydro-2H-pyran-2-yl}acetate (P4)

To a solution of sodium ethoxide in EtOH [generated via cautious addition of sodium metal (1.1 g, 48 mmol) to EtOH (100 mL)] was added a solution of ethyl (2E,6R,7S)-6-[bis(cyclopentylmethyl)amino]-7-hydroxy-7-[4-(trifluoromethyl)phenyl]hept-2-enoate (20 g, 40 mmol) in EtOH (100 mL). The mixture was heated to reflux for 1 hour and then cooled to room temperature. Concentrated H₂SO₄ (3 mL, 50 mmol) was added and the mixture was heated to reflux for 18 hours. After cooling to room temperature, the mixture was concentrated to approximately half the original volume under reduced pressure. The mixture was neutralized with saturated aqueous NaHCO₃ solution and extracted with heptane. The organic layer was dried over MgSO₄ and the solvent removed under reduced pressure. The residue was purified by silica gel chromatography (Gradient: 0% to 5% EtOAc in heptane) to provide ethyl {(2S,5R,6S)-5-[bis(cyclopentylmethyl)amino]-6-[4-(trifluoromethyl)phenyl]tetrahydro-2H-pyran-2-yl}acetate (P3) as a thick oil (13.26 g, 66%). The relative stereochemistry of P3 was assigned on the basis of NMR studies, specifically the observation of NOE signals between the methine proton adjacent to the aryl group and the methine adjacent to the ethyl acetyl moiety, and the 9.9 Hz coupling constant (indicating a diaxial relationship) between the methine proton adjacent to the tertiary amine and the proton adjacent to the aryl group. ¹H NMR (400 MHz, CDCl₃) δ 0.74-0.99 (m, 5H), 1.07-1.16 (m, 2H), 1.18 (t, J=7.1 Hz, 3H), 1.22-1.58 (m, 10H), 1.61-1.75 (m, 3H), 1.85-1.94 (m, 1H), 1.95-2.02 (m, 1H), 2.05 (dd, J=12.5, 9.4 Hz, 2H), 2.25 (dd, J=12.6, 5.4 Hz, 2H), 2.40 (dd, J=15.3, 6.3 Hz, 1H), 2.56 (dd, J=15.3, 6.6 Hz, 1H), 2.73 (ddd, J=11.7, 10.0, 3.8 Hz, 1H), 3.85 (m, 1H), 4.08 (m, 2H), 4.32 (d, J=9.9 Hz, 1H), 7.44 (d, J=8.2 Hz, 2H), 7.52 (d, J=8.4 Hz, 2H). A smaller quantity of the isomeric ethyl {(2R,5R,6S)-5-[bis(cyclopentylmethyl)amino]-6-[4-(trifluoromethyl)phenyl]tetrahydro-2H-pyran-2-yl}acetate (P4) was also isolated (1.7 g, 9%). ¹H NMR (400 MHz, CDCl₃) δ 0.82-0.91 (m, 2H), 0.94-1.04 (m, 2H), 1.16-1.24 (m, 2H), 1.23 (t, J=7.1 Hz, 3H), 1.33-1.63 (m, 10H), 1.70-1.91 (m, 5H), 1.97-2.07 (m, 1H), 2.12 (dd, J=12.6, 9.5 Hz, 2H), 2.32 (dd, J=12.6, 5.2 Hz, 2H), 2.60 (dd, J=14.3, 6.8 Hz, 1H), 2.81-2.88 (m, 1H), 2.92 (dd, J=14.3, 8.2 Hz, 1H), 4.07-4.20 (m, 2H), 4.44-4.50 (m, 1H), 4.57 (d, J=10.0 Hz, 1H), 7.53 (AB quartet, J_{AB}=8.3 Hz, Δν_{AB}=23.3 Hz, 4H).

### Preparation 5

### Methyl 2-{(2R,4S,5S)-5-amino-4-[4-(trifluoromethyl)phenyl]tetrahydro-2H-pyran-2-yl}acetate, hydrochloride salt (P5)

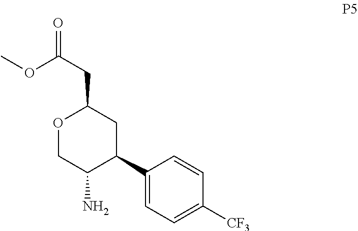

### Step 1. Synthesis of tert-butyl (4S)-4-{(1S)-3-methoxy-3-oxo-1-[4-(trifluoromethyl)phenyl]propyl}-2,2-dimethyl-1,3-oxazolidine-3-carboxylate

Copper iodide (16.9 g, 87.6 mmol) was suspended in THF (100 mL) and cooled to −78° C. To the cold suspension was added 4-(trifluoromethyl)phenylmagnesium bromide (1 M in THF, 175 mL, 175 mmol) drop-wise. The mixture was warmed to 0° C. and stirred for 2 hours. The mixture was cooled back to −78° C. and trimethylsilyl chloride (22.2 mL, 175 mmol) was added followed by tert-butyl (4S)-4-[(1E)-3-methoxy-3-oxoprop-1-en-1-yl]-2,2-dimethyl-1,3-oxazolidine-3-carboxylate (5.0 g, 18 mmol, see Tetrahedron: Asymmetry 2006, 3170-3178) as a solution in THF (50 mL). The reaction was allowed to warm slowly to room temperature overnight. The reaction was poured into 9:1 saturated aqueous NH₄Cl: concentrated NH₄OH (500 mL) and extracted with tert-butyl methyl ether and EtOAc. The combined organic layers were washed with 1 M aqueous NaOH, dried over MgSO₄, filtered, and the solvent removed under reduced pressure. The residue was purified by silica gel chromatography (Gradient: 10% to 30% EtOAc in heptane) to provide the title compound as a thick oil (7.3 g, 97%). LCMS m/z 432.2 (M+1). ¹H NMR (400 MHz, CDCl₃) δ 1.19-1.32 (m, 3H), 1.35-1.71 (m, 12H), 2.84 (d, J=7.3 Hz, 2H), 3.54 (s, 3H), 3.58-4.03 (m, 4H), 7.28-7.41 (m, 2H), 7.54 (dd, J=6.2, 3.1 Hz, 2H).

### Step 2. Synthesis of tert-butyl (4S)-4-{(1S)-3-hydroxy-1-[4-(trifluoromethyl)phenyl]propyl}-2,2-dimethyl-1,3-oxazolidine-3-carboxylate

LiAlH₄ (0.806 g, 20.2 mmol) was suspended in THF (20 mL) and cooled to 0° C. tert-Butyl (4S)-4-{(1S)-3-methoxy-3-oxo-1-[4-(trifluoromethyl)phenyl]propyl}-2,2-dimethyl-1,3-oxazolidine-3-carboxylate (5.8 g, 13 mmol) was added drop-wise as a solution in THF (50 mL). The mixture was warmed to room temperature and stirred for 45 minutes. The reaction was quenched by the sequential addition of water (0.9 mL), 15% aqueous NaOH (0.9 mL) and water (2.7 mL). The mixture was stirred for 30 minutes and then filtered. The solids were rinsed with THF and the combined filtrates were concentrated under reduced pressure. The residue was purified by silica gel chromatography (Gradient: 10% to 30% EtOAc in heptane) to provide the title compound as a thick oil (5.4 g, quant.). ¹H NMR (400 MHz, CDCl₃) δ 1.14-1.69 (m, 15H), 1.98-2.14 (m, 2H), 3.24-3.53 (m, 3H), 3.53-3.64 (m, 1H), 3.71-3.84 (m, 1H), 3.88-3.98 (m, 1H), 4.03-4.15 (m, 1H), 7.30-7.42 (m, 2H), 7.45-7.63 (m, 2H).

### Step 3. Synthesis of tert-butyl (4S)-4-{(1S,3E)-5-methoxy-5-oxo-1-[4-(trifluoromethyl)phenyl]pent-3-en-1-yl}-2,2-dimethyl-1,3-oxazolidine-3-carboxylate

To a cooled, −78° C., solution of oxalyl chloride (2.64 mL, 30.2 mmol) in CH₂Cl₂ (25 mL) was added a solution of dimethyl sulfoxide (4.34 mL, 60.5 mmol) in CH₂Cl₂ (10 mL). After stirring for 15 minutes, a solution of tert-butyl (4S)-4-{(1S)-3-hydroxy-1-[4-(trifluoromethyl)phenyl]propyl}-2,2-dimethyl-1,3-oxazolidine-3-carboxylate (5.42 g, 13.4 mmol) in CH₂Cl₂ (25 mL) was added drop-wise over 15 minutes. The mixture was stirred for an additional 15 minutes, and then a solution of triethylamine (10.3 mL, 73.9 mmol) in CH₂Cl₂ (10 mL) was added drop-wise. The resulting mixture was stirred for 1 hour at −78° C. Methyl (triphenylphosphoranylidene)acetate (9.0 g, 27 mmol) was added in one portion and the mixture was warmed to room temperature. After stirring for 3 hours at room temperature, the reaction was poured into water (100 mL) and extracted with CH₂Cl₂. The combined organic layers were dried over MgSO₄, filtered, and the solvent was removed under reduced pressure. The residue was purified by silica gel chromatography (Gradient: 10% to 30% EtOAc in heptane) to provide the title compound as a thick oil (3.4 g, 55%). ¹H NMR (400 MHz, CDCl₃) δ 1.24 (br s, 3H), 1.36-1.57 (m, 10H), 1.67 (br s, 1H), 2.59-2.82 (m, 2H), 3.50 (dt, J=10.0, 5.1 Hz, 1H), 3.63 (s, 3H), 3.70-3.82 (m, 1H), 3.86 (dd, J=9.7, 1.6 Hz, 1H), 3.92-4.02 (m, 1H), 4.07-4.16 (m, 1H), 5.78 (d, J=15.1 Hz, 1H), 6.73 (dt, J=15.5, 7.1 Hz, 1H), 7.25-7.41 (m, 2H), 7.44-7.58 (m, 2H).

### Step 4. Synthesis of methyl (2E,5S,6S)-6-[tert-butoxycarbonyl)amino]-7-hydroxy-5-[4-(trifluoromethyl)phenyl]hept-2-enoate

tert-Butyl (4S)-4-{(1S,3E)-5-methoxy-5-oxo-1-[4-(trifluoromethyl)phenyl]pent-3-en-1-yl}-2,2-dimethyl-1,3-oxazolidine-3-carboxylate (3.4 g, 7.4 mmol) was dissolved in MeOH (75 mL). p-Toluenesulfonic acid monohydrate (0.147 g, 0.743 mmol) was added and the mixture was stirred at room temperature for 14 hours. The reaction was partitioned between EtOAc and 1:1 saturated aqueous NaCl solution: saturated aqueous NaHCO₃ solution. The aqueous layer was extracted with EtOAc and the combined organic layers dried over MgSO₄. After filtration, the solvent was removed under reduced pressure and the residue was purified by silica gel chromatography (Gradient: 30% to 50% EtOAc in heptane) to provide the title compound as white solid (1.6 g, 52%). ¹H NMR (400 MHz, CDCl₃) δ 1.3 (s, 9H), 1.9 (br s, 1H), 2.6-2.8 (m, 2H), 3.2-3.3 (m, 1H), 3.5-3.6 (m, 2H), 3.65 (s, 3H), 3.8-3.9 (m, 1H), 4.3-4.4 (m, 1H), 5.78 (d, 1H), 6.73 (dt, 1H), 7.3 (d, 2H), 7.5 (d, 2H).

### Step 5. Synthesis of methyl 2-{(2R,4S,5S)-5-(tert-butoxycarbonylamino)-4-[4-(trifluoromethyl)phenyl]tetrahydro-2H-pyran-2-yl}acetate

Methyl (2E,5S,6S)-6-[(tert-butoxycarbonyl)amino]-7-hydroxy-5-[4-(trifluoromethyl)phenyl]hept-2-enoate (300 mg, 0.719 mmol) was dissolved in THF (5 mL) and treated with tetrabutylammonium fluoride (1 M in THF, 1.0 mL, 1 mmol). The reaction was stirred at room temperature for 1 hour and then partitioned between EtOAc and 1 M aqueous HCl. The organic layer was washed with water and dried over MgSO₄. After filtration, the solvent was removed under reduced pressure and the residue was purified by silica gel chromatography (Gradient: 10% to 30% EtOAc in heptane) to provide the title compound as a white solid (225 mg, 75%). ¹H NMR (400 MHz, CDCl₃) δ 1.23 (s, 9H), 1.54-1.70 (m, 1H), 1.94 (ddd, J=13.4, 3.9, 2.1 Hz, 1H), 2.43 (dd, J=15.5, 5.6 Hz, 1H), 2.60 (dd, J=15.5, 7.4 Hz, 1H), 2.65-2.78 (m, 1H), 3.23 (t, J=10.8 Hz, 1H), 3.67 (s, 3H), 3.79-3.94 (m, 2H), 4.06 (br s, 1H), 4.14 (dd, J=11.1, 4.8 Hz, 1H), 7.33 (d, J=8.1 Hz, 2H), 7.55 (d, J=8.2 Hz, 2H).

### Step 6. Synthesis of methyl 2-{(2R,4S,5S)-5-amino-4-[4-(trifluoromethyl)phenyl]tetrahydro-2H-pyran-2-yl}acetate, hydrochloride salt (P5)

Methyl 2-{(2R,4S,5S)-5-(tert-butoxycarbonylamino)-4-[4-(trifluoromethyl)phenyl]tetrahydro-2H-pyran-2-yl}acetate (500 mg, 1.2 mmol) was dissolved in CH₂Cl₂ (10 mL) and treated with HCl in 1,4-dioxane (3.0 mL, 4 M, 12 mmol). The mixture was stirred for 18 hours at room temperature and the solvent removed under reduced pressure to provide the title compound as a white solid (424 mg, quant). The reaction was repeated, and MS data was obtained on a sample LCMS m/z 318.0 (M+1).

### Preparation 6

### Methyl {(3S,4R)-4-amino-3-[6-(trifluoromethyl)pyridin-3-yl]cyclohexyl}acetate (P6)

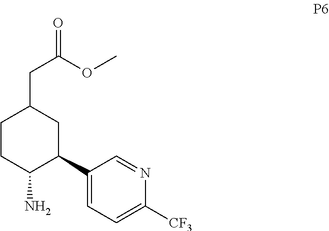

### Step 1. Synthesis of 2-nitro-1-[6-(trifluoromethyl)pyridin-3-yl]ethanol

tert-Butanol (99%, 33.2 mL, 349 mmol) and nitromethane (99%, 4.76 mL, 87.2 mmol) were added to a solution of 6-(trifluoromethyl)nicotinaldehyde (10.2 g, 58.2 mmol) in THF (100 mL), and the solution was cooled to 0° C. Potassium tert-butoxide (99%, 659 mg, 5.81 mmol) was added, and the reaction was allowed to slowly warm to room temperature. After 3 days, solvents were removed in vacuo and the residue was partitioned between water (100 mL) and EtOAc. The organic layer was washed with water, then dried over MgSO₄, filtered and concentrated in vacuo. Purification via silica gel chromatography (Eluant: 20% EtOAc in heptane) afforded the product as an off-white solid (8.8 g, 64%). APCI m/z 237.0 (M+1). ¹H NMR (400 MHz, CDCl₃) δ 3.15 (br s, 1H), 4.60 (dd, half of ABX pattern, J=14, 3.6 Hz, 1H), 4.65 (dd, half of ABX pattern, J=13.9, 8.7 Hz, 1H), 5.65 (br d, J=8.6 Hz, 1H), 7.77 (d, J=8.1 Hz, 1H), 8.01 (dd, J=8.1, 2.2 Hz, 1H), 8.79 (d, J=1.9 Hz, 1H).

### Step 2. Synthesis of 5-[(E)-2-nitrovinyl]-2-(trifluoromethyl)pyridine

Acetic anhydride (98%, 3.59 mL, 37.2 mmol) was added to a solution of 2-nitro-1-[6-(trifluoromethyl)pyridin-3-yl]ethanol (8.8 g, 37 mmol) in CH₂Cl₂ (100 mL). N,N-Dimethylpyridin-4-amine (99%, 230 mg, 1.86 mmol) was added, and the reaction was stirred at room temperature for 2 hours. Saturated aqueous NaHCO₃ solution (50 mL) was added, and the organic layer was dried over MgSO₄, filtered and concentrated in vacuo. Purification by silica gel chromatography (Gradient: 10% to 20% EtOAc in heptane) provided the product as a light yellow solid (6.3 g, 78%). ¹H NMR (400 MHz, CDCl₃) δ 7.68 (d, J=13.9 Hz, 1H), 7.81 (d, J=8.1 Hz, 1H), 8.04-8.08 (m, 1H), 8.05 (d, J=14.1 Hz, 1H), 8.91-8.92 (m, 1H).

### Step 3. Synthesis of trans-4-nitro-3-[6-(trifluoromethyl)pyridin-3-yl]cyclohexanone

A solution of 5-[(E)-2-nitrovinyl]-2-(trifluoromethyl)pyridine (6.3 g, 29 mmol) and 2-(trimethylsilyl)oxy-1,3-butadiene (10.0 mL, 57.6 mmol) in toluene (25 mL) was heated to 120° C. in a sealed vessel. After 18 hours, the reaction was cooled and treated with 1 M aqueous HCl (15 mL) and MeOH; this mixture was allowed to stir at room temperature for 30 minutes. The reaction was then extracted with EtOAc and the combined organic layers were dried over MgSO₄, filtered and concentrated under reduced pressure. Purification via silica gel chromatography (Gradient: 10% to 50% EtOAc in heptane) provided the product as a thick oil (7.4 g, 89%). APCI m/z 289.0 (M+1). ¹H NMR (400 MHz, CDCl₃), selected peaks: δ 3.85 (ddd, J=12.3, 10.8, 5.5 Hz, 1H), 5.12 (ddd, J=10.6, 10.6, 3.8 Hz, 1H), 7.71 (br d, J=8.1 Hz, 1H), 7.78 (dd, J=8.2, 2.2 Hz, 1H), 8.67 (d, J=2.1 Hz, 1H).

### Step 4. Synthesis of methyl (2E/2Z)-{3,4-trans-4-nitro-3-[6-(trifluoromethyl)pyridin-3-yl]cyclohexylidene}acetate

Methyl (triphenylphosphoranylidene)acetate (12.9 g, 38.6 mmol) was added to a solution of trans-4-nitro-3-[6-(trifluoromethyl)pyridin-3-yl]cyclohexanone (7.4 g, 26 mmol) in toluene (50 mL), and the resulting solution was heated at 100° C. for 2 hours. After cooling to room temperature, the reaction was concentrated in vacuo and diluted with diethyl ether (50 mL). After stirring for 5 minutes, the mixture was filtered; the solid was washed with additional diethyl ether, and the combined filtrates were concentrated in vacuo. Purification using silica gel chromatography (Gradient: 10% to 30% EtOAc in heptane) afforded the product as a mixture of E and Z olefin isomers (6.46 g, 73%).

### Step 5. Synthesis of methyl {(3S,4R)-4-amino-3-[6-(trifluoromethyl)pyridin-3-yl]cyclohexylidene}acetate

AcOH (50 mL, 870 mmol) and zinc (50 g, 760 mmol) were added to a solution of methyl (2E12Z)-{3,4-trans-4-nitro-3-[6-(trifluoromethyl)pyridin-3-yl]cyclohexylidene}acetate (6.4 g, 19 mmol) in THF (20 mL). The reaction was stirred at room temperature for 3 hours, then heated to 75° C. for 2.5 hours. The reaction was cooled to room temperature and filtered; the collected solids were washed with EtOAc and MeOH. The combined filtrates were concentrated in vacuo, and the resulting residue was dissolved in EtOAc, washed with saturated aqueous NaHCO₃ solution, dried over MgSO₄, filtered and concentrated. Silica gel chromatography (Gradient 0% to 10% MeOH in EtOAc) was used to separate the E and Z isomers of the product. The less polar isomer was subjected to chiral chromatography to separate its enantiomers (Column: Chiralcel OJ-H, 5 μm; Mobile phase: 90/10 CO₂/MeOH with 0.2% isopropylamine modifier). The later-eluting enantiomer was obtained as a solid (509 mg, 9%). The stereochemistry about the double bond is unknown; assignment of the absolute configuration was made on the basis of the greater biological activity of a product derived from this enantiomer versus the corresponding analogue prepared using the earlier-eluting enantiomer. ¹H NMR (400 MHz, CDCl₃) δ 0.99 (br s, 2H), 1.40-1.51 (m, 1H), 2.05-2.23 (m, 2H), 2.38-2.44 (m, 1H), 2.44-2.53 (dd, J=13, 13 Hz, 1H), 2.59 (ddd, J=12, 10, 4 Hz, 1H), 3.17 (ddd, J=10.6, 10.2, 3.8 Hz, 1H), 3.73 (s, 3H), 4.01 (br d, J=14 Hz, 1H), 5.71 (s, 1H), 7.69 (d, J=8.0 Hz, 1H), 7.77 (br d, J=8.0 Hz, 1H), 8.65 (s, 1H).

### Step 6. Synthesis of methyl {(3S,4R)-4-amino-3-[6-(trifluoromethyl)pyridin-3-yl]cyclohexyl}acetate (P6)

Rhodium on carbon (5%, loading factor 0.485 mmol/g, 165 mg, 0.0800 mmol) and methyl {(3S,4R)-4-amino-3-[6-(trifluoromethyl)pyridin-3-yl]cyclohexylidene}acetate (500 mg, 1.59 mmol) were combined in MeOH (10 mL), and hydrogenated for 18 hours under 30 psi of hydrogen. The reaction was filtered and solvent was removed in vacuo to provide the product as an oil (500 mg, 99%). This material, presumed to be a mixture of diastereomers at the center bearing the acetate group, was used without additional purification. APCI m/z 317.1 (M+1).

### Preparation 7

### Methyl [(1R,3S,4R)-4-amino-3-(4-chlorophenyl)cyclohexyl]acetate (P7)

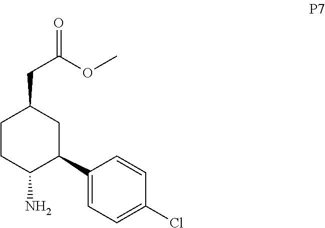

### Step 1. Synthesis of (1S,3S,4R)-4-[(tert-butoxycarbonyl)amino]-3-(4-chlorophenyl)cyclohexyl methanesulfonate

Triethylamine (1.34 mL, 9.64 mmol) was added to a solution of tert-butyl [(1R,2S,4S)-2-(4-chlorophenyl)-4-hydroxycyclohexyl]carbamate (prepared in the same manner as tert-butyl {(1R,2S,4S)-4-hydroxy-2-[4-(trifluoromethyl)phenyl]cyclohexyl}carbamate in Preparation 1, but employing 4-chlorophenylmagnesium chloride rather than 4-(trifluoromethyl)phenylmagnesium bromide) (2.10 g, 6.44 mmol) in CH₂Cl₂ (30 mL), and the flask was cooled in an ice bath. After addition of methanesulfonyl chloride (0.701 mL, 9.02 mmol), the reaction was stirred at 0° C. for 3 hours. Saturated aqueous NaHCO₃ solution (100 mL) was then added, and the mixture was extracted with CH₂Cl₂ (2×150 mL). The combined organic layers were washed with saturated aqueous NaHCO₃ solution (100 mL) and with water (100 mL), then dried over MgSO₄. Filtration and removal of solvent under reduced pressure provided the product as a white foam (2.25 g, 86%).

### Step 2. Synthesis of diethyl [(1R,3S,4R)-4-[(tert-butoxycarbonyl)amino]-3-(4-chlorophenyl)cyclohexyl]malonate

A solution of potassium tert-butoxide (1 M in THF, 20.8 mL, 20.8 mmol) was added drop-wise over 10 minutes to an ice-cooled solution of diethyl malonate (3.95 mL, 26.0 mmol) in N,N-dimethylformamide (10 mL). The reaction was allowed to warm to room temperature, and was stirred for 1.5 hours. A solution of (1S,3S,4R)-4-[(tert-butoxycarbonyl)amino]-3-(4-chlorophenyl)cyclohexyl methanesulfonate (2.10 g, 5.20 mmol) in N,N-dimethylformamide (40 mL) was added drop-wise over 20 minutes, and the reaction mixture was heated to 40° C. for 20 minutes, then to 100° C., and stirred at that temperature for 14 hours. The reaction was then cooled and treated with saturated aqueous NaHCO₃ solution (100 mL). After extraction with EtOAc (3×100 mL), the combined organic layers were washed with saturated aqueous NaCl solution (100 mL), dried over MgSO₄, filtered and concentrated in vacuo. The residue was precipitated from EtOAc/heptane to provide the product as a white solid; subsequent reprecipitation from the mother liquor provided a second crop of the product, also as a white solid (total: 1.66 g, 68%). ¹H NMR (400 MHz, DMSO-d₆) δ 1.12 (t, J=7.1 Hz, 3H), 1.17 (t, J=7.1 Hz, 3H), 1.18 (s, 9H), 1.23-1.45 (m, 3H), 1.63-1.72 (m, 2H), 1.83-1.90 (m, 1H), 1.97-2.08 (m, 1H), 2.52-2.60 (m, 1H), 3.29 (d, J=8.6 Hz, 1H), 3.39-3.49 (m, 1H), 4.04-4.15 (m, 4H), 6.55 (d, J=9.2 Hz, 1H), 7.24 (AB quartet, J_{AB}=8.5 Hz, Δν_{AB}=39.0 Hz, 4H).

### Step 3. Synthesis of methyl [(1R,3S,4R)-4-amino-3-(4-chlorophenyl)cyclohexyl]acetate (P7)

Concentrated HCl (12 M, 50 mL, 600 mmol) was added drop-wise to a mixture of diethyl [(1R,3S,4R)-4-[(tert-butoxycarbonyl)amino]-3-(4-chlorophenyl)cyclohexyl]malonate (9.00 g, 19.2 mmol) in water (50 mL). The resulting heterogeneous mixture was stirred at 114° C. for 22 hours. The reaction mixture was concentrated in vacuo; the residue was then admixed with CH₂Cl₂ (500 mL) and treated drop-wise with half-saturated aqueous NaHCO₃ solution (100 mL). After 30 minutes of stirring, additional CH₂Cl₂ (1 L) was added to solubilize remaining solids. The organic layer was washed with half-saturated aqueous NaHCO₃ solution (2×100 mL), then dried over MgSO₄, filtered and concentrated in vacuo. The resulting material was mixed with MeOH (100 mL) and concentrated under reduced pressure: this was carried out three times. The resulting material was characterized as a mixture of methyl ester P7 and the corresponding carboxylic acid. LCMS m/z 282.4 (M+1) and m/z 268.4, 270.4 (M+1). MeOH (100 mL) was added to this mixture, and the resulting solution was treated drop-wise with concentrated H₂SO₄ (0.1 mL), then stirred for 18 hours at room temperature. Solvent was removed under reduced pressure, and the residue was dissolved in CH₂Cl₂ (500 mL). After slow addition of half-saturated NaHCO₃ solution (100 mL), the aqueous layer was extracted with CH₂Cl₂ (3×150 mL), and the combined organic layers were washed with half-saturated NaHCO₃ solution (100 mL), dried over MgSO₄, filtered and concentrated in vacuo to provide the product as a pale brown solid (4.60 g, 85%). This material was used without additional purification. LCMS m/z 282.4 (M+1). ¹H NMR (400 MHz, CDCl₃) δ 1.16-1.41 (m, 5H), 1.81-1.91 (m, 2H), 1.92-2.04 (m, 2H), 2.19-2.31 (m, 2H), 2.29-2.36 (m, 1H), 2.80 (ddd, J=10.5, 10.5, 3.9 Hz, 1H), 3.66 (s, 3H), 7.16 (d, J=8.4 Hz, 2H), 7.29 (d, J=8.4 Hz, 2H).

### Preparation 8

### Methyl [(1R,3S,4R)-4-amino-3-(4-fluorophenyl)cyclohexyl]acetate (P8)

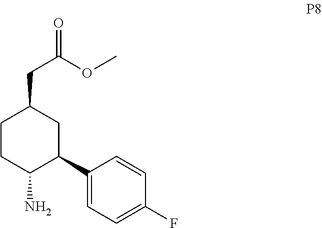

Dimethyl [(1R,3S,4R)-4-[(tert-butoxycarbonyl)amino]-3-(4-fluorophenyl)cyclohexyl]malonate (prepared in a manner analogous to that described for diethyl {(1R,3S,4R)-4-[(tert-butoxycarbonyl)amino]-3-[4-(trifluoromethyl)phenyl]cyclohexyl}malonate in Preparation 1) (680 mg, 1.61 mmol) was treated with MeOH (2 mL) and aqueous HCl (6 M, 7 mL). The reaction was heated at 100° C. for 18 hours, then diluted with additional MeOH (15 mL) and heated for an additional 24 hours. After concentration in vacuo, the residue was dissolved in MeOH (10 mL), treated with a few drops of concentrated H₂SO₄, and heated to reflux for 18 hours. After cooling, solvents were removed in vacuo and the residue was partitioned between tert-butyl methyl ether and 1 M aqueous NaOH. The organic layer was dried over MgSO₄, filtered and concentrated to provide the product as a solid. This material was determined by NMR and LCMS to be a 1:1 mixture of P8 with the corresponding dimethyl malonate analogue (corrected yield for P8: 180 mg, 42%). LCMS m/z 266.4 (M+1) and m/z 324.4 (M+1). ¹H NMR (400 MHz, CDCl₃) (reported NMR data represents an equimolar mixture of P8 and the dimethyl malonate analogue) δ 1.16-1.44 (m, 10H), 1.78-1.90 (m, 4H), 1.92-2.06 (m, 3H), 2.19-2.38 (m, 5H), 2.80 (ddd, J=10.5, 10.5, 3.9 Hz, 2H), 3.22 (d, J=8.9 Hz, 1H), 3.66 (s, 3H), 3.70 (s, 3H), 3.74 (s, 3H), 6.98-7.03 (m, 4H), 7.15-7.20 (m, 4H).

### Preparation 9

### Methyl {3,4-trans-4-[(3-methylbutyl)(3,3,3-trifluoropropyl)amino]-3-[4-(trifluoromethyl)phenyl]cyclohexyl}acetate (P9)

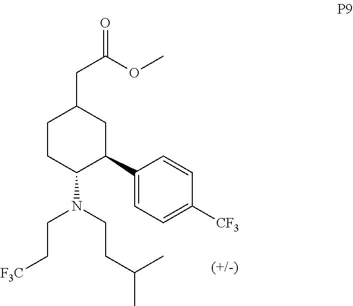

### Step 1. Synthesis of methyl {3,4-trans-4-amino-3-[4-(trifluoromethyl)phenyl]cyclohexyl}acetate

A solution of ethyl (2E/2Z)-{3,4-trans-4-nitro-3-[4-(trifluoromethyl)phenyl]cyclohexylidene}acetate (prepared in a manner analogous to that described for methyl (2E12Z)-{3,4-trans-4-nitro-3-[6-(trifluoromethyl)pyridin-3-yl]cyclohexylidene}acetate in Preparation 6) (4.5 g, 13 mmol) in EtOAc (40 mL) was added to a suspension of palladium on carbon (10%, wet with 50% water, 2.0 g) in EtOAc (10 mL), and the mixture was hydrogenated at 50 psi for 18 hours. After the addition of Celite, the mixture was filtered and the filter cake was washed with EtOAc and MeOH. The combined filtrates were concentrated under reduced pressure and the residue was chromatographed on silica gel (Eluants: EtOAc, then EtOAc containing 1% NH₄OH and 10% MeOH) to afford the product as an oil (970 mg, 24%). This material was judged by ¹H NMR to be a roughly 2:1 mixture of diastereomers at the center bearing the methyl acetate. LCMS m/z 316.1 (M+1).

### Step 2. Synthesis of methyl {3,4-trans-4-[(3-methylbutyl)(3,3,3-trifluoropropyl)amino]-3-[4-(trifluoromethyl)phenyl]cyclohexyl}acetate (P9)

3-Methylbutanal (0.191 mL, 1.78 mmol) and MgSO₄ (99%, 868 mg, 7.14 mmol) were added to a solution of methyl {3,4-trans-4-amino-3-[4-(trifluoromethyl)phenyl]cyclohexyl}acetate (450 mg, 1.43 mmol) in 1,2-dichloroethane (10 mL), and the reaction was stirred at room temperature for 4 hours. After filtration and washing of solids with CH₂Cl₂, the combined filtrates were concentrated in vacuo to an oil, which was dissolved in toluene (50 mL) and concentrated again. The resulting material was dissolved in 1,2-dichloroethane (10 mL), treated with sodium triacetoxyborohydride (98%, 365 mg, 1.69 mmol), and allowed to stir for 18 hours. At this point, 3,3,3-trifluoropropanal (0.267 mL, 2.86 mmol) was added, followed by AcOH (0.165 mL, 2.86 mmol) and additional sodium triacetoxyborohydride (98%, 406 mg, 1.88 mmol). After 3 hours, the reaction was quenched with saturated aqueous NaHCO₃ solution. The aqueous layer was extracted with CH₂Cl₂, and the combined organic layers were dried over MgSO₄, filtered and concentrated in vacuo. Purification via silica gel chromatography (Eluant: 5% EtOAc in heptane) provided the product as a thick oil (426 mg, 62%). This material was judged by ¹H NMR to be a roughly 2:1 mixture of diastereomers at the center bearing the methyl acetate. APCI m/z 482.4 (M+1). ¹H NMR (400 MHz, CDCl₃) δ 0.76-0.79 (m, 6H), 0.85-1.51 (m, 6H), 1.63-1.85 (m, 4H), 1.89-2.03 (m, 2H), 2.18-2.53 (m, 5H), 2.65-2.87 (m, 3H), 3.70 and 3.66 (2 singlets, 3H), 7.23 (d, J=8.0 Hz, 2H), 7.53 (d, J=8.0 Hz, 2H).

### Examples and Methods

### Example 1

### Method A

### Synthesis of {(1R,3S,4R)-4-[bis(cyclopentylmethyl)amino-]-3-[4-(trifluoromethyl)phenyl]cyclohexyl}acetic acid (1)

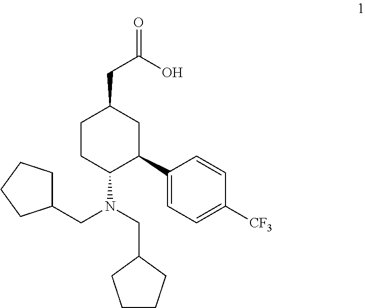

### Step 1. Synthesis of methyl {(1R,3S,4R)-4-[bis(cyclopentylmethyl)amino]-3-[4-(trifluoromethyl)phenyl]cyclohexyl}acetate

Methyl {(1R,3S,4R)-4-amino-3-[4-(trifluoromethyl)phenyl]cyclohexyl}acetate (P1) (7.08 g, 22.4 mmol) was dissolved in 1,2-dichloroethane (100 mL) and treated with cyclopentanecarbaldehyde (7.9 mL, 74 mmol) followed by sodium triacetoxyborohydride (17 g, 79 mmol). The reaction mixture was stirred at room temperature for 5 hours. The mixture was diluted with CH₂Cl₂ and quenched with saturated aqueous NaHCO₃ solution. The organic layer was separated and dried over MgSO₄, filtered and concentrated under reduced pressure. The residue was purified by silica gel chromatography (Gradient: 0% to 50% EtOAc in heptane) and further by precipitation from MeOH/water to provide the title compound as a white solid (8.06 g, 75%). APCI m/z 480.2 (M+1).

### Step 2. Synthesis of {(1R,3S,4R)-4-[bis(cyclopentylmethyl)amino]-3-[4-(trifluoromethyl)phenyl]cyclohexyl}acetic acid (1)

Methyl {(1R,3S,4R)-4-[bis(cyclopentylmethyl)amino]-3-[4-(trifluoromethyl)phenyl]cyclohexyl}acetate (8.06 g, 16.8 mmol) was dissolved in THF (85 mL) and MeOH (85 mL), and treated with 1 M aqueous NaOH (85 mL). The resulting mixture was stirred at room temperature for 18 hours then neutralized with 1 M aqueous HCl. The mixture was extracted three times with EtOAc and the combined organic layers washed with saturated aqueous NaCl solution, dried over MgSO₄ and filtered. The solvent was removed under reduced pressure to provide the title compound as a white foamy solid (7.2 g, 92%).

¹H NMR (400 MHz, CDCl₃) δ 0.71-0.80 (m, 2H), 0.92-1.02 (m, 2H), 1.04-1.21 (m, 4H), 1.31-1.63 (m, 11H), 1.75-1.99 (m, 6H), 2.04-2.19 (m, 4H), 2.22 (dd, J=12.7, 5.1 Hz, 2H), 2.72-2.85 (m, 2H), 7.26 (d, 2H; assumed, partially obscured by solvent peak), 7.49 (d, J=8.2 Hz, 2H). The reaction was repeated and MS data was obtained on the product. LCMS m/z 466.3 (M+1).

### Example 2

### Method B

### Synthesis of {(1R,3S,4R)-4-[benzyl(cyclopentylmethyl)amino]-3-[4-(trifluoromethyl)phenyl]cyclohexyl}acetic acid (2)

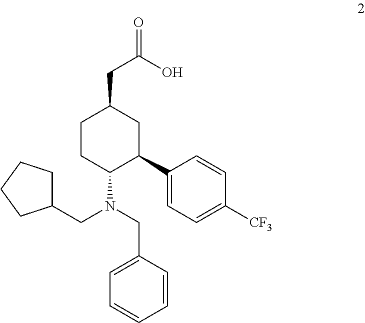

### Step 1. Synthesis of methyl {(1R,3S,4R)-4-[(cyclopentylmethyl)amino]-3-[4-(trifluoromethyl)phenyl]cyclohexyl}acetate

Methyl {(1R,3S,4R)-4-amino-3-[4-(trifluoromethyl)phenyl]cyclohexyl}acetate (P1) (4.00 g, 12.7 mmol) was dissolved in MeOH (9 mL) and treated with cyclopentanecarbaldehyde (97%, 1.54 mL, 14.0 mmol). The reaction mixture was stirred at room temperature for 4 hours. The mixture was cooled to 0° C. and sodium borohydride (1.44 g, 38.1 mmol) was added in one portion. When LCMS data indicated that the reaction was complete, the reaction was quenched with saturated aqueous NaHCO₃ solution and extracted twice with CH₂Cl₂. The organic layer was dried over MgSO₄, filtered and concentrated under reduced pressure. The residue was purified by silica gel chromatography (Gradient: 30% to 100% EtOAc in heptane) to provide the product as a white solid (4.81 g, 95%). LCMS m/z 398.5 (M+1).

### Step 2. Synthesis of methyl {(1R,3S,4R)-4-[benzyl(cyclopentylmethyl)amino]-3-[4-(trifluoromethyl)phenyl]cyclohexyl}acetate

Methyl {(1R,3S,4R)-4-[(cyclopentylmethyl)amino]-3-[4-(trifluoromethyl)phenyl]cyclohexyl}acetate (200 mg, 0.503 mmol) was dissolved in THF (5 mL), cooled to 0° C., and treated with AcOH (58 μL, 1.0 mmol) and benzaldehyde (76 μL, 0.75 mmol). Sodium triacetoxyborohydride (163 mg, 0.754 mmol) was added, and the reaction was allowed to warm to room temperature. After 4 hours, the mixture was quenched with saturated aqueous NaHCO₃ solution and extracted with CH₂Cl₂. The combined organic layers were dried over MgSO₄, filtered and concentrated under reduced pressure. Purification by silica gel chromatography (Gradient: 0% to 10% EtOAc in heptane) provided the title compound as a colorless oil (190 mg, 78%). APCI m/z 488.2 (M+1).

### Step 3. Synthesis of {(1R,3S,4R)-4-[benzyl(cyclopentylmethyl)amino]-3-[4-(trifluoromethyl)phenyl]cyclohexyl}acetic acid (2)

Methyl {(1R,3S,4R)-4-[benzyl(cyclopentylmethyl)amino]-3-[4-(trifluoromethyl)phenyl]cyclohexyl}acetate (190 mg, 0.390 mmol) was dissolved in a mixture of THF (2 mL) and MeOH (2 mL), then treated with 1 M aqueous NaOH (2 mL). The resulting mixture was stirred at room temperature for 18 hours, then made slightly acidic by addition of 1 M aqueous HCl. The mixture was extracted with CH₂Cl₂ and the combined organic layers were concentrated in vacuo, yielding a semi-solid. This material was suspended in diethyl ether and filtered; the filtrate was concentrated in vacuo to provide the title compound as a white foam (135 mg, 73%). APCI m/z 474.2 (M+1). ¹H NMR (400 MHz, CDCl₃) δ 0.64-0.74 (m, 1H), 0.91-1.01 (m, 1H), 1.07-1.50 (m, 8H), 1.53-1.63 (m, 1H), 1.83-2.02 (m, 4H), 2.07-2.15 (m, 2H), 2.18-2.32 (m, 2H), 2.36 (dd, J=12.3, 4.9 Hz, 1H), 2.80-2.90 (m, 2H), 3.26 (d, J=14.1 Hz, 1H), 3.71 (d, J=14.0 Hz, 1H), 6.84-6.88 (m, 2H), 7.10-7.18 (m, 5H), 7.48 (d, J=8.0 Hz, 2H).

### Example 3

### Method C

### Synthesis of {(2S,5R,6S)-5-[bis(cyclopentylmethyl)amino]-6-[4-(trifluoromethyl)phenyl]tetrahydro-2H-pyran-2-yl}acetic acid (3)

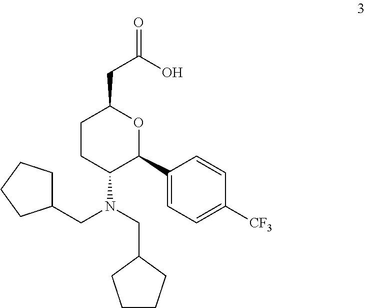

Ethyl {(2S,5R,6S)-5-[bis(cyclopentylmethyl)amino]-6-[4-(trifluoromethyl)phenyl]tetrahydro-2H-pyran-2-yl}acetate (P3) (13.2 g, 26.6 mmol) was dissolved in THF (100 mL) and MeOH (100 mL) and treated with aqueous NaOH (1 M, 133 mL, 133 mmol). The mixture was warmed to 50° C. for 1 hour. The reaction was cooled to room temperature and then made acidic (pH 4-5) with concentrated HCl. The mixture was extracted with tert-butyl methyl ether and the organic layer dried over Na₂SO₄. The solvent was removed under reduced pressure to provide the title compound as a white foamy solid (10.3 g, 83%). APCI m/z 468.2 (M+1). ¹H NMR (400 MHz, CDCl₃) δ 0.71-0.85 (m, 2H), 0.85-0.99 (m, 2H), 1.10 (td, J=12.1, 7.1 Hz, 2H), 1.24-1.58 (m, 12H), 1.59-1.76 (m, 3H), 1.80-1.99 (m, 2H), 2.03 (dd, J=12.5, 9.6 Hz, 2H), 2.24 (dd, J=12.6, 5.3 Hz, 2H), 2.36-2.65 (m, 2H), 2.74 (t, J=10.0 Hz, 1H), 3.82 (br s, 1H), 4.34 (d, J=9.7 Hz, 1H), 7.43 (d, J=8.3 Hz, 2H), 7.52 (d, J=7.9 Hz, 2H).

### Example 4

### Method D

### Synthesis of {(2S,5R,6S)-5-[(cyclopentylmethyl)(isobutyl)amino]-6-[4-(trifluoromethyl)phenyl]tetrahydro-2H-pyran-2-yl}acetic acid (4)

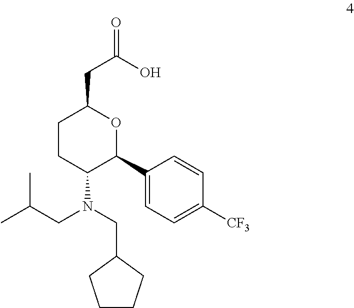

### Step 1. Synthesis of ethyl (2E,6R)-6-(dibenzylamino)-7-hydroxyhept-2-enoate

Ethyl (2E,6R)-6-amino-7-hydroxyhept-2-enoate (2.01 g, 9 mmol) and benzaldehyde (2.34 mL, 22.5 mmol) were combined in CH₂Cl₂ (50 mL). Sodium triacetoxyborohydride (5.84 g, 27 mmol) was added and the mixture was stirred at room temperature. After 4 hours, the reaction was quenched with saturated aqueous NaHCO₃ solution. The mixture was extracted with CH₂Cl₂ and the combined organic layers were dried over MgSO₄, filtered and concentrated under reduced pressure. The residue was purified by silica gel chromatography (Gradient: 5% to 50% EtOAc in heptane) to provide the title compound as a thick oil (2.36 g, 70%). LCMS m/z 368.1 (M+1). ¹H NMR (400 MHz, CDCl₃) δ 1.28 (t, J=7.2 Hz, 3H), 1.32-1.43 (m, 1H), 1.77-1.92 (m, 1H), 2.03-2.26 (m, 2H), 2.68-2.85 (m, 1H), 2.93 (d, J=8.3 Hz, 1H), 3.40 (d, J=13.2 Hz, 2H), 3.43-3.56 (m, 2H), 3.78 (d, J=13.3 Hz, 2H), 4.18 (q, J=7.1 Hz, 2H), 5.78 (dt, J=15.6, 1.4 Hz, 1H), 6.89 (dt, J=15.7, 6.8 Hz, 1H), 7.16-7.36 (m, 10H).

### Step 2. Synthesis of ethyl (2E,6R)-6-(dibenzylamino)-7-oxohept-2-enoate

Ethyl (2E,6R)-6-(dibenzylamino)-7-hydroxyhept-2-enoate was oxidized to the corresponding aldehyde using the method described for preparation of ethyl (2E,6R)-6-[bis(cyclopentylmethyl)amino]-7-oxohept-2-enoate (P2) in Preparation 2. The product was obtained as a thick oil, which was used without purification (1.2 g, quant).

### Step 3. Synthesis of ethyl (2E,6R,7S)-6-(dibenzylamino)-7-hydroxy-7-[4-(trifluoromethyl)phenyl]hept-2-enoate

To a cooled, −78° C., solution of 4-(trifluoromethyl)phenylmagnesium bromide in THF (20 mL, 0.4 M, 8 mmol) was added a solution of ethyl (2E,6R)-6-(dibenzylamino)-7-oxohept-2-enoate (1.2 g, 3.3 mmol) in THF (30 mL) drop-wise over 30 minutes. The reaction was stirred at −78° C. for 10 minutes and then quenched with saturated aqueous NH₄Cl solution. The mixture was warmed to room temperature and extracted with CH₂Cl₂. The combined organic layers were dried over MgSO₄, filtered, and the solvent removed under reduced pressure. The residue was purified by silica gel chromatography (Eluant: 10% EtOAc in heptane) to provide the title compound as a thick oil (1.28 g, 76%). APCI m/z 512.1 (M+1). ¹H NMR (400 MHz, CDCl₃) δ 1.25 (t, J=7.1 Hz, 3H), 1.39-1.49 (m, 1H), 1.84-1.99 (m, 1H), 2.00-2.09 (m, 1H), 2.11 (d, J=4.7 Hz, 1H), 2.34-2.49 (m, 1H), 2.75-2.87 (m, 1H), 3.60 (d, J=14.0 Hz, 2H), 3.86 (d, J=13.8 Hz, 2H), 4.13 (q, J=7.1 Hz, 2H), 5.15 (t, J=4.0 Hz, 1H), 5.63 (dt, J=15.6, 1.4 Hz, 1H), 6.81 (dt, J=15.6, 6.8 Hz, 1H), 7.17-7.33 (m, 12H), 7.50 (d, J=8.1 Hz, 2H).

### Step 4. Synthesis of ethyl {(2S,5R,6S)-5-(dibenzylamino)-6-[4-(trifluoromethyl)phenyl]tetrahydro-2H-pyran-2-yl}acetate

To a solution of sodium ethoxide in EtOH [generated via addition of sodium hydride (40% suspension in oil, 0.053 g, 1.3 mmol) to EtOH (1 mL)] was added a solution of ethyl (2E,6R,7S)-6-(dibenzylamino)-7-hydroxy-7-[4-(trifluoromethyl)phenyl]hept-2-enoate (0.67 g, 2.5 mmol) in EtOH (5 mL). The mixture was heated to 60° C. for 1 hour and then cooled to room temperature. Concentrated H₂SO₄ was added to make the solution acidic (pH ˜1) and the mixture was heated to reflux for 3 hours. The mixture was cooled to room temperature and neutralized with saturated aqueous NaHCO₃ solution and extracted with tert-butyl methyl ether. The organic layer was dried over MgSO₄ and the solvent removed under reduced pressure. The residue was purified by silica gel chromatography (Gradient: 5% to 10% EtOAc in heptane) to provide the title compound as a thick oil (517 mg, 77%). LCMS m/z 512.5 (M+1). ¹H NMR (400 MHz, CDCl₃) δ 1.16 (t, J=7.1 Hz, 3H), 1.34-1.49 (m, 1H), 1.72-1.87 (m, 1H), 1.88-2.02 (m, 1H), 2.23 (dq, J=12.9, 3.5 Hz, 1H), 2.37 (dd, J=15.4, 6.4 Hz, 1H), 2.52 (dd, J=15.4, 6.5 Hz, 1H), 2.70 (ddd, J=12.1, 9.9, 3.8 Hz, 1H), 3.41 (d, J=13.9 Hz, 2H), 3.67 (d, J=13.9 Hz, 2H), 3.88 (m, 1H), 4.00-4.14 (m, 2H), 4.48 (d, J=9.9 Hz, 1H), 6.84-6.95 (m, 4H), 7.09-7.18 (m, 6H), 7.21 (d, J=8.2 Hz, 2H), 7.51 (d, J=8.3 Hz, 2H).

### Step 5. Synthesis of ethyl {(2S,5R,6S)-5-amino-6-[4-(trifluoromethyl)phenyl]tetrahydro-2H-pyran-2-yl}acetate

Ethyl {(2S,5R,6S)-5-(dibenzylamino)-6-[4-(trifluoromethyl)phenyl]tetrahydro-2H-pyran-2-yl}acetate (400 mg, 0.78 mmol), Pd(OH)₂ (10% on carbon, 70 mg) and ammonium formate (1.26 g, 19.6 mmol) were combined in MeOH (10 mL). The mixture was stirred at room temperature for 18 hours and then filtered. The solvent was removed under reduced pressure and the residue suspended in CH₂Cl₂. Filtration and removal of the solvent under reduced pressure provided the title compound as a thick oil (246 mg, 95%). LCMS m/z 332.5 (M+1). ¹H NMR (400 MHz, CDCl₃) δ 1.19 (t, J=7.1 Hz, 3H), 1.43-1.64 (m, 2H), 1.82-1.90 (m, 1H), 2.03-2.13 (m, 1H), 2.45 (dd, J=15.3, 6.1 Hz, 1H), 2.60 (dd, J=15.3, 6.8 Hz, 1H), 2.69 (ddd, J=10.7, 9.2, 4.0 Hz, 1H), 3.88-3.96 (m, 1H), 3.97 (d, J=9.1 Hz, 1H), 4.09 (2 quartets, J=7.1 Hz, 2H), 7.47 (d, J=8.1 Hz, 2H), 7.58 (d, J=8.0 Hz, 2H).

### Step 6. Synthesis of methyl {(2S,5R,6S)-5-[(cyclopentylmethyl)amino]-6-[4-(trifluoromethyl)phenyl]tetrahydro-2H-pyran-2-yl}acetate

Methyl {(2S,5R,6S)-5-amino-6-[4-(trifluoromethyl)phenyl]tetrahydro-2H-pyran-2-yl}acetate was reacted with cyclopentanecarbaldehyde using the method described for preparation of methyl {(1R,3S,4R)-4-[(cyclopentylmethyl)amino]-3-[4-(trifluoromethyl)phenyl]cyclohexyl}acetate in Example 2. The product was obtained as a thick oil (190 mg, 76%). APCI m/z 400.1 (M+1). ¹H NMR (400 MHz, CDCl₃) δ 0.62 (br s, 1H), 0.72-0.98 (m, 2H), 1.31-1.59 (m, 8H), 1.62-1.77 (m, 1H), 1.79-1.92 (m, 1H), 2.03 (dd, J=11.2, 7.2 Hz, 1H), 2.12-2.23 (m, 1H), 2.34-2.52 (m, 3H), 2.60 (dd, J=15.4, 6.8 Hz, 1H), 3.62 (s, 3H), 3.91 (m, 1H), 4.11 (d, J=9.2 Hz, 1H), 7.47 (d, J=8.2 Hz, 2H), 7.57 (d, J=8.5 Hz, 2H).

### Step 7. Synthesis of methyl {(2S,5R,6S)-5-[(cyclopentylmethyl)(isobutyl) amino]-6-[4-(trifluoromethyl)phenyl]tetrahydro-2H-pyran-2-yl}acetate

Methyl {(2S,5R,6S)-5-[(cyclopentylmethyl)amino]-6-[4-(trifluoromethyl)phenyl]tetrahydro-2H-pyran-2-yl}acetate (140 mg, 0.35 mmol) was dissolved in THF (3 mL). 2-Methylpropanal (0.48 mL, 0.52 mmol) was added followed by sodium triacetoxyborohydride (114 mg, 0.52 mmol). The mixture was stirred at room temperature for 18 hours and then quenched with saturated aqueous NaHCO₃ solution. The mixture was extracted with CH₂Cl₂ and the combined organic layers were dried over MgSO₄, filtered and concentrated under reduced pressure to provide the title compound, which was used without further purification (160 mg, quant). APCI m/z 456.2 (M+1). ¹H NMR (400 MHz, CDCl₃) δ 0.43 (d, J=6.4 Hz, 3H), 0.67 (d, J=6.6 Hz, 3H), 1.08-1.28 (m, 2H), 1.29-1.60 (m, 7H), 1.60-1.76 (m, 2H), 1.78-1.94 (m, 3H), 1.94-2.06 (m, 2H), 2.11 (dd, J=13.2, 5.2 Hz, 1H), 2.24 (dd, J=12.6, 5.5 Hz, 1H), 2.40 (dd, J=15.6, 6.4 Hz, 1H), 2.57 (dd, J=15.6, 6.6 Hz, 1H), 2.64-2.75 (m, 1H), 3.61 (s, 3H), 3.77-3.90 (m, 1H), 4.32 (d, J=9.8 Hz, 1H), 7.43 (d, J=7.7 Hz, 2H), 7.51 (d, J=8.2 Hz, 2H).

### Step 8. Synthesis of {(2S,5R,6S)-5-[(cyclopentylmethyl)(isobutyl)amino]-6-[4-(trifluoromethyl)phenyl]tetrahydro-2H-pyran-2-yl}acetic acid (4)

Methyl {(2S,5R,6S)-5-[(cyclopentylmethyl)(isobutyl)amino]-6-[4-(trifluoromethyl)phenyl]tetrahydro-2H-pyran-2-yl}acetate (159 mg, 0.35 mmol) was dissolved in THF (1 mL) and MeOH (1 mL) and treated with 1 M aqueous NaOH (3 mL, 3 mmol). The mixture was stirred at room temperature for 3.5 hours. The reaction was then made acidic (pH 4-5) with 1 M aqueous HCl. The mixture was extracted with CH₂Cl₂ and the organic layer dried over Na₂SO₄. The solvent was removed under reduced pressure and the residue purified by silica gel chromatography (Gradient: 0% to 5% MeOH in CH₂Cl₂) to provide the title compound as a white foamy solid (108 mg, 70%). APCI m/z 440.3 (M−1). ¹H NMR (400 MHz, CDCl₃) δ 0.43 (d, J=6.5 Hz, 3H), 0.68 (d, J=6.6 Hz, 3H), 0.73-0.86 (m, 1H), 0.87-1.01 (m, 2H), 1.07-1.60 (m, 8H), 1.61-1.78 (m, 2H), 1.81-1.94 (m, 2H), 1.96-2.07 (m, 2H), 2.12 (dd, J=12.8, 5.1 Hz, 1H), 2.24 (dd, J=12.4, 5.2 Hz, 1H), 2.42-2.65 (m, 2H), 2.67-2.81 (m, 1H), 3.75-3.93 (m, 1H), 4.36 (d, J=9.9 Hz, 1H), 7.43 (d, J=8.4 Hz, 2H), 7.53 (m, J=8.2 Hz, 2H).

### Example 5

### Method E

### Synthesis of 2-{(2R,4S,5S)-5-[bis(cyclopentylmethyl)amino]-4-[4-(trifluoromethyl)phenyl]tetrahydro-2H-pyran-2-yl}acetic acid (5)

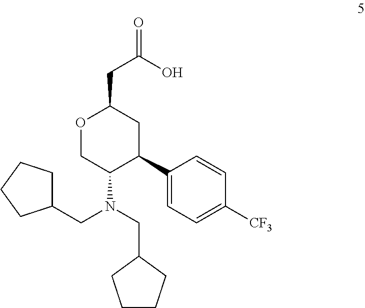

### Step 1. Synthesis of methyl 2-{(2R,4S,5S)-5-[bis(cyclopentylmethyl)amino]-4-[4-(trifluoromethyl)phenyl]tetrahydro-2H-pyran-2-yl}acetate

Methyl 2-{(2R,4S,5S)-5-amino-4-[4-(trifluoromethyl)phenyl]tetrahydro-2H-pyran-2-yl}acetate, hydrochloride salt (P5) (100 mg, 0.283 mmol), cyclopentanecarbaldehyde (0.125 mL, 1.13 mmol), AcOH (0.065 mL, 1.13 mmol) and sodium triacetoxyborohydride (184 mg, 0.849 mmol) were combined in THF (5 mL) and stirred at room temperature. After 6 hours the reaction was quenched with saturated aqueous NaHCO₃ solution and the mixture was extracted with CH₂Cl₂. The combined organic layers were dried over MgSO₄ and the solvent removed under reduced pressure. The residue was purified by silica gel chromatography (Gradient: 5% to 20% EtOAc in heptane) to provide the title compound as a white solid (144 mg, quant). APCI m/z 482.4 (M+1). ¹H NMR (400 MHz, CDCl₃) δ 0.72-0.85 (m, 2H), 0.90-1.03 (m, 2H), 1.15-1.29 (m, 2H), 1.29-1.61 (m, 8H), 1.80 (dt, J=14.7, 7.4 Hz, 2H), 1.88 (ddd, J=13.2, 3.6, 2.2 Hz, 1H), 2.12 (dd, J=12.6, 8.6 Hz, 2H), 2.23 (dd, J=12.7, 6.3 Hz, 2H), 2.39 (dd, J=15.2, 5.4 Hz, 1H), 2.55 (dd, J=15.3, 7.79 Hz, 1H), 2.93-3.11 (m, 2H), 3.37-3.50 (m, 2H), 3.66 (s, 3H), 3.76-3.89 (m, 2H), 4.08 (dd, J=11.3, 4.0 Hz, 2H), 7.31 (d, J=7.9 Hz, 2H), 7.51 (d, J=8.5 Hz, 2H).

### Step 2. Synthesis of 2-{(2R,4S,5S)-5-[bis(cyclopentylmethyl)amino]-4-[4-(trifluoromethyl)phenyl]tetrahydro-2H-pyran-2-yl}acetic acid (5)

Methyl 2-{(2R,4S,5S)-5-[bis(cyclopentylmethyl)amino]-4-[4-(trifluoromethyl)phenyl]tetrahydro-2H-pyran-2-yl}acetate (136 mg, 0.28 mmol) was dissolved in THF (2 mL) and MeOH (2 mL). Aqueous NaOH (1 M, 2 mL, 2 mmol) was added and the mixture was warmed to 50° C. for 1 hour. The reaction was cooled to room temperature and made acidic (pH 4-5) with 1 M aqueous HCl. The mixture was extracted with CH₂Cl₂ and the organic layer dried over MgSO₄. The solvent was removed under reduced pressure to provide the title compound as a white solid (119 mg, 90%). LCMS m/z 468.6 (M+1). ¹H NMR (400 MHz, CDCl₃) δ 0.71-0.88 (m, 2H), 0.90-1.08 (m, 2H), 1.15-1.32 (m, 2H), 1.32-1.68 (m, 8H), 1.74-1.86 (m, 2H), 1.91 (d, J=13.0 Hz, 1H), 2.06-2.19 (m, 2H), 2.24 (dd, J=12.4, 6.2 Hz, 2H), 2.48 (dd, J=15.8, 4.7 Hz, 1H), 2.57 (dd, J=16.0, 7.3 Hz, 1H), 2.92-3.18 (m, 2H), 3.48 (t, J=10.6 Hz, 2H), 3.73-3.90 (m, 2H), 4.13 (d, J=9.7 Hz, 2H), 7.32 (d, J=8.1 Hz, 2H), 7.52 (d, J=7.6 Hz, 2H).

### Example 6

### Synthesis of [(1R,3S,4R)-4-[bis(cyclopentylmethyl)amino]-3-(4-chlorophenyl)cyclohexyl]acetic acid (6)

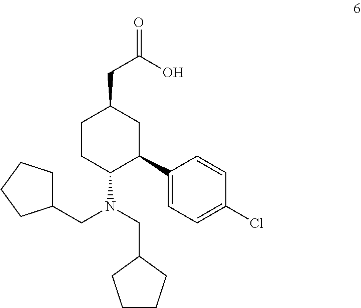

### Step 1. Synthesis of methyl [(1R,3S,4R)-4-[bis(cyclopentylmethyl)amino]-3-(4-chlorophenyl)cyclohexyl]acetate

Methyl [(1R,3S,4R)-4-amino-3-(4-chlorophenyl)cyclohexyl]acetate (P7) was converted to the product using conditions similar to those described for the synthesis of methyl {(1R,3S,4R)-4-[bis(cyclopentylmethyl)amino]-3-[4-(trifluoromethyl)phenyl]cyclohexyl}acetate in Example 1. The product was obtained as an oil (5.60 g, 77%). LCMS m/z 446.6, 448.6 (M+1). ¹H NMR (400 MHz, CDCl₃) δ 0.77-0.86 (m, 2H), 0.94-1.04 (m, 2H), 1.07-1.28 (m, 4H), 1.31-1.63 (m, 11H), 1.76-1.99 (m, 6H), 2.05-2.12 (m, 2H), 2.14-2.27 (m, 4H), 2.65-2.81 (m, 2H), 3.64 (s, 3H), 7.09 (d, J=8.5 Hz, 2H), 7.21 (d, J=8.4 Hz, 2H).

### Step 2. Synthesis of [(1R,3S,4R)-4-[bis(cyclopentylmethyl)amino]-3-(4-chlorophenyl)cyclohexyl]acetic acid (6)

Methyl [(1R,3S,4R)-4-[bis(cyclopentylmethyl)amino]-3-(4-chlorophenyl)cyclohexyl]acetate was converted to the product using conditions similar to those employed for synthesis of {(1R,3S,4R)-4-[bis(cyclopentylmethyl)amino]-3-[4-(trifluoromethyl)phenyl]cyclohexyl}acetic acid (1) in Example 1, except that the product was subjected to two purifications using silica gel chromatography (Gradient: 5% to 100% [10% (2 N ammonia in MeOH) in CH₂Cl₂] in CH₂Cl₂; then gradient: 0% to 20% MeOH in CH₂Cl₂). The product was obtained as a white solid (350 mg, 90%). APCI m/z 432.2, 434.3 (M+1). ¹H NMR (400 MHz, CDCl₃) δ 0.76-0.86 (m, 2H), 0.95-1.04 (m, 2H), 1.06-1.28 (m, 4H), 1.33-1.64 (m, 11H), 1.77-1.99 (m, 6H), 2.07-2.27 (m, 6H), 2.66-2.84 (m, 2H), 7.09 (d, J=8.4 Hz, 2H), 7.21 (d, J=8.4 Hz, 2H).

### Examples 7 and 8

### Synthesis of (2R)-2-{(1R,3S,4R)-4-[bis(3-methylbutyl)amino]-3-[4-(trifluoromethyl)phenyl]cyclohexyl}propanoic acid (7) and (2S)-2-{(1R,3S,4R)-4-[bis(3-methylbutyl)amino]-3-[4-(trifluoromethyl)phenyl]cyclohexyl}propanoic acid (8)

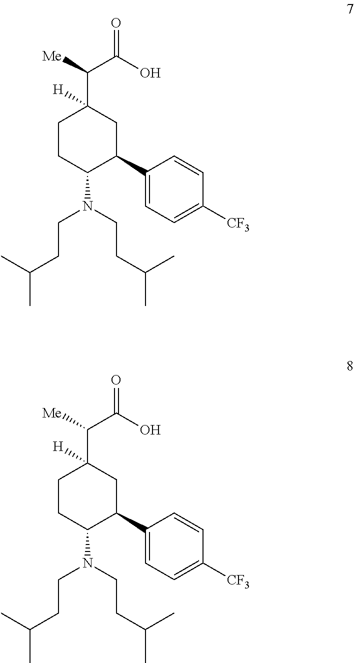

Step 1. Synthesis of diethyl {(1R,3S,4R)-4-amino-3-[4-(trifluoromethyl)phenyl]cyclohexyl}malonate. Trifluoroacetic acid (0.3 mL) was added drop-wise to a 0° C. solution of diethyl {(1R,3S,4R)-4-[(tert-butoxycarbonyl)amino]-3-[4-(trifluoromethyl)phenyl]cyclohexyl}malonate (100 mg, 0.199 mmol) in CH₂Cl₂ (1.2 mL). After 30 minutes, the reaction was warmed to room temperature and allowed to stir for 1 hour. The reaction was concentrated in vacuo and then azeotroped with toluene; the residue was dissolved in CH₂Cl₂ (10 mL) and washed with saturated aqueous NaHCO₃ solution (5 mL) and with saturated aqueous NaCl solution (5 mL). After drying over Na₂SO₄, the organic layer was filtered and concentrated under reduced pressure to provide the product as a gum (80 mg, 100%). LCMS m/z 402.2 (M+1).

### Step 2. Synthesis of diethyl {(1R,3S,4R)-4-[bis(3-methylbutyl)amino]-3-[4-(trifluoromethyl)phenyl]cyclohexyl}malonate

3-Methylbutanal (65 μL, 0.60 mmol), AcOH (34 μL, 0.60 mmol) and sodium triacetoxyborohydride (95%, 134 mg, 0.601 mmol) were added to a solution of diethyl {(1R,3S,4R)-4-amino-3-[4-(trifluoromethyl)phenyl]cyclohexyl}malonate (80.3 mg, 0.200 mmol) in THF (2 mL), and the reaction was stirred at room temperature for 18 hours. After addition of saturated aqueous NaHCO₃ solution, the mixture was extracted with EtOAc (3×10 mL) and the combined organic layers were washed with saturated aqueous NaCl solution, dried over Na₂SO₄, filtered and concentrated in vacuo. Purification via silica gel chromatography (Gradient: 0% to 60% EtOAc in heptane) afforded the product as a colorless oil (103 mg, 95%). LCMS m/z 542.4 (M+1).

### Step 3. Synthesis of diethyl {(1R,3S,4R)-4-[bis(3-methylbutyl)amino]-3-[4-(trifluoromethyl)phenyl]cyclohexyl}(methyl)malonate

A solution of diethyl {(1R,3S,4R)-4-[bis(3-methylbutyl)amino]-3-[4-(trifluoromethyl)phenyl]cyclohexyl}malonate (100 mg, 0.185 mmol) in THF (1.0 mL) was added drop-wise to a stirred suspension of sodium hydride (60% in mineral oil, 14.8 mg, 0.37 mmol) in THF (1 mL). After 1 hour, methyl iodide (23 μL, 0.37 mmol) was added, and the reaction was allowed to proceed for 5 hours. Saturated aqueous NH₄Cl solution was added, and the mixture was extracted with EtOAc (3×10 mL). The combined organic layers were washed with saturated aqueous NaCl solution, dried over Na₂SO₄, filtered and concentrated under reduced pressure to provide the product as a colorless gum (103 mg, 100%). LCMS m/z 556.8 (M+1).

### Step 5. Synthesis of (2R)-2-{(1R,3S,4R)-4-[bis(3-methylbutyl)amino]-3-[4-(trifluoromethyl)phenyl]cyclohexyl}propanoic acid (7) and (2S)-2-{(1R,3S,4R)-4-[bis(3-methylbutyl(amino]-3-[4-(trifluoromethyl)phenyl]cyclohexyl}propanoic acid (8)

Diethyl {(1R,3S,4R)-4-[bis(3-methylbutyl(amino]-3-[4-(trifluoromethyl)phenyl]cyclohexyl}(methyl)malonate (95 mg, 0.17 mmol) was dissolved in ethanol (1.5 mL) and treated with aqueous NaOH solution (15% by weight, 0.66 mL, 3.4 mmol). The mixture was heated to 110° C. for 7 hours, then cooled and brought to pH 4 with 1 N aqueous NaHSO₄ solution. After extraction with EtOAc (5×15 mL), the combined organic layers were dried over Na₂SO₄, filtered and concentrated. The residue was dissolved in dimethyl sulfoxide (1 mL), treated with aqueous HCl (6 M, 1 mL) and heated to 95° C. for 3 hours. After removal of solvent in vacuo, the remaining material was mixed with water (5 mL) and taken to pH 4 by addition of 1 N aqueous NaHSO₄ solution. The mixture was extracted with EtOAc (5×15 mL), dried over Na₂SO₄, filtered and concentrated under reduced pressure. Purification by silica gel chromatography (Gradient: 0% to 10% MeOH in CH₂Cl₂) effected a separation of the diastereomers, to provide the first-eluting isomer (2R)-2-{(1R,3S,4R)-4-[bis(3-methylbutyl(amino]-3-[4-(trifluoromethyl)phenyl]cyclohexyl}propanoic acid (7) as a white solid (14 mg, 18%). LCMS m/z 456.6 (M+1). ¹H NMR (400 MHz, CDCl₃) δ 0.77 (br d, J=6.6 Hz, 12H), 0.91-1.43 (m, 9H), 1.16 (d, J=7.0 Hz, 3H), 1.68-1.79 (m, 1H), 1.81-2.04 (m, 3H), 2.18-2.41 (m, 5H), 2.68-2.82 (m, 2H), 7.24 (d, J=7.8 Hz, 2H), 7.50 (d, J=8.0 Hz, 2H). (2S)-2-{(1R,3S,4R)-4-[Bis(3-methylbutyl)amino]-3-[4-(trifluoromethyl)phenyl]cyclohexyl}propanoic acid (8), the later-eluting isomer, was also obtained as a white solid (8 mg, 11%). LCMS m/z 456.6 (M+1). ¹H NMR (400 MHz, CDCl₃) δ 0.77 (br d, J=6.6 Hz, 12H), 0.92-1.44 (m, 9H), 1.14 (d, J=7.0 Hz, 3H), 1.70-1.81 (m, 1H), 1.85-1.94 (m, 2H), 1.96-2.02 (m, 1H), 2.18-2.28 (m, 2H), 2.29-2.41 (m, 3H), 2.69-2.81 (m, 2H), 7.25 (d, J=8.1 Hz, 2H), 7.51 (d, J=8.1 Hz, 2H). Stereochemistry at the methyl group was arbitrarily assigned.

### Method F

### Synthesis of N,N-disubstituted (4-amino-3-aryl-cyclohexyl)acetic acids by reductive amination of the mono-N-substituted ester congeners followed by ester hydrolysis

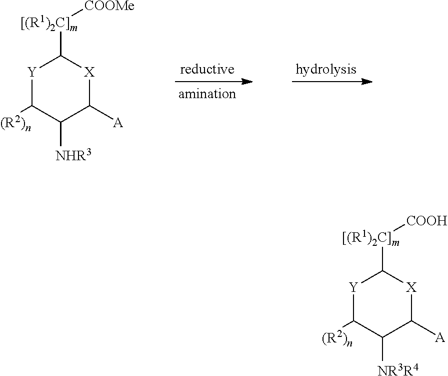

Solutions of the appropriate N-substituted methyl (4-amino-3-aryl-cyclohexyl)acetates in CH₂Cl₂ (0.5 M, 0.35 mL, 0.18 mmol) were added to solutions of sodium triacetoxyborohydride in CH₂Cl₂ (0.55 M, 1.0 mL, 0.55 mmol). After addition of solutions of the requisite aldehydes in CH₂Cl₂ (0.5 M, 0.25 mL, 0.12 mmol), the reactions were treated with glacial acetic acid (15 μL, 0.26 mmol) and shaken at room temperature for 18 hours. Solvent was removed, and MeOH (3 mL) was added to each reaction. The mixtures were applied to columns packed with Dowex in MeOH, and neutral impurities were washed off with MeOH (5×5 mL). Elution with 30% diethylamine in MeOH (2×5 mL) provided product fractions; the solvent was removed, and the amino esters were subjected to ester hydrolysis with LiOH in a mixture of THF, MeOH and water, in a procedure similar to that described for the final step of Example 2. Final products were purified by reversed-phase HPLC, using a gradient composed of mobile phases 0.1% TFA in water and 0.1% TFA in acetonitrile, and one of the following columns: 1) YMC-pack ODS-AQ, 5 μm; 2) Phenomenex Luna C₁₈, 5 μm; 3) Kromasil Eternity-5-C₁₈, 5 μm; 4) Agella Venusil ASB C₁₈, 5 μm.

Biological data for Examples 1-8 is given in Table 1. The structures of additional Examples, with preparative information, characterization data and biological activity, are provided in Tables 2 and 3. In some cases, hydrochloride salts were tested; these salts were generally prepared by dissolving the neutral compound in diethyl ether or CH₂Cl₂ and adding a solution of HCl in diethyl ether or 1,4-dioxane. The hydrochloride salt can be isolated via filtration or removal of solvents. Purification of compounds in Table 2 was generally carried out via silica gel chromatography, with solvent gradients employing MeOH in CH₂Cl₂ or MeOH in EtOAc.

TABLE 1
Biological data for Examples 1-8

| Example # | Aβ 42B IC₅₀ (nM)¹ |
|---|---|
| 1 | 229² |
| 2 | 139 |
| 3 | 357² |
| 4 | 608 |
| 5 | 777 |
| 6 | 719² |
| 7 | 1750 |
| 8 | 1580 |

¹IC₅₀ values represent the geometric mean of 2 determinations, unless otherwise indicated.
²IC₅₀ values represent the geometric mean of 6-14 determinations.

TABLE 2

| | | | Aβ 42B | | |
|---|---|---|---|---|---|
| | | Method | IC₅₀ | | |
| | | of | (nM) | | |
| | | preparation; | (Geometric | | ¹H NMR (400 MHz, CDCl₃), δ |
| | | starting | Mean of | | (ppm); Mass spectrum, LCMS |
| Ex | | material | 2-8 Deter- | | observed ion m/z (M + 1) |
| # | Structure | (s) | minations) | IUPAC Name | (unless otherwise indicated) |
| 9 | 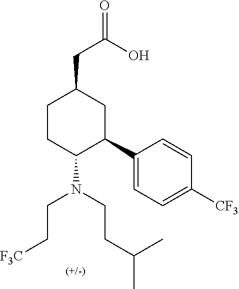 | C¹; P9 | 618 | {(1R,3S,4R)/ (1S,3R,4S)-4- [(3-methylbutyl) (3,3,3- trifluoropropyl) amino]-3-[4- (trifluoromethyl) phenyl]cyclohexyl} acetic acid, hydrochloride salt | 0.77 (d, J = 6.6 Hz, 3H), 0.78 (d, J = 6.6 Hz, 3H), 0.81-1.44 and 1.64-1.86 (multiplets, 8H), 1.89-2.03 (m, 4H), 2.19-2.37 (m, 4H), 2.46 (ddd, J = 13.3, 10.7, 5.0 Hz, 1H), 2.65-2.81 (m, 3H), 7.23 (d, J = 7.9 Hz, 2H), 7.52 (d, J = 7.9 Hz, 2H); 468.2⁴ |
| 10 | 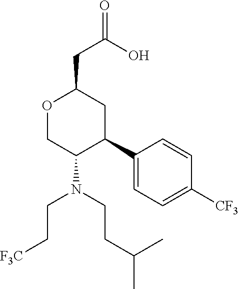 | B; P5 | 4770 | 2-{(2R,4S,5S)- 5-[(3- methylbutyl)(3,3,3- trifluoropropyl) amino]-4-[4- (trifluoromethyl) phenyl]tetrahydro- 2H-pyran-2-yl} acetic acid, hydrochloride salt | 0.78 (d, J = 6.6 Hz, 3H), 0.79 (d, J = 6.6 Hz, 3H), 0.98-1.15 (m, 2H), 1.23-1.33 (m, 1H), 1.62- 1.72 (m, 1H), 1.77-1.88 (m, 1H), 1.93-2.04 (m, 2H), 2.32- 2.46 (m, 2H), 2.50 (dd, J = 15.6, 5.1 Hz, 1H), 2.56-2.65 (m, 2H), 2.72 (ddd, J = 13.4, 10.8, 5.2 Hz, 1H), 2.93-3.06 (m, 2H), 3.44-3.51 (m, 1H), 3.85-3.92 (m, 1H), 4.11 (dd, J = 11.2, 3.8 Hz, 1H), 7.31 (d, J = 8.1 Hz, 2H), 7.57 (d, J = 8.2 Hz, 2H); APCI m/z 470.1 (M + 1)⁴ |
| 11 | 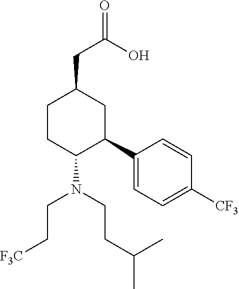 | B; P1 | 470 | {(1R,3S,4R)-4- [(3-methylbutyl) (3,3,3- trifluoropropyl) amino]-3-[4- (trifluoromethyl) phenyl]cyclohexyl} acetic acid, hydrochloride salt | 0.75-0.79 (m, 6H), 0.86-1.46 (m, 6H), 1.66-1.87 (m, 2H), 1.90-2.03 (m, 4H), 2.21-2.56 (m, 5H), 2.64-2.84 (m, 3H), 7.23 (d, J = 8 Hz, 2H), 7.52 (d, J = 8 Hz, 2H); 468.1⁴ |
| 12 | 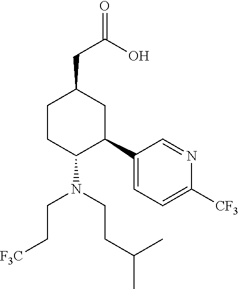 | B¹; P6 | 1370 | {(1R,3S,4R)-4- [(3-methylbutyl) (3,3,3-trifluoro- propyl)amino]-3-[6- (trifluoromethyl) pyridin-3-yl] cyclohexyl}acetic acid | 0.78 (d, J = 6.6 Hz, 6H), 0.89- 0.99 (m, 1H), 1.04-1.13 (m, 1H), 1.17-1.28 (m, 2H), 1.29- 1.49 (m, 2H), 1.68-1.87 (m, 2H), 1.94-2.05 (m, 4H), 2.23- 2.38 (m, 4H), 2.50 (ddd, J = 13, 11, 5 Hz, 1H), 2.66-2.75 (m, 2H), 2.81-2.89 (m, 1H), 7.59- 7.64 (m, 2H), 8.51 (br s, 1H); APCI m/z 467.2 (M − 1) |
| 13 | 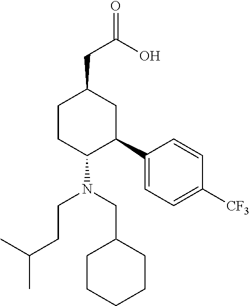 | B; P1 | 236 | {(1R,3S,4R)-4- [(cyclohexylmethyl) (3-methylbutyl) amino]-3-[4- (trifluoromethyl) phenyl]cyclohexyl} acetic acid | 0.18-0.29 (m, 1H), 0.59-0.70 (m, 1H), 0.77-0.90 (m, 1H), 0.83 (d, J = 6.5 Hz, 3H), 0.85 (d, J = 6.4 Hz, 3H), 0.93-1.21 (m, 6H), 1.26-1.45 (m, 5H), 1.53- 1.65 (m, 3H), 1.88 (dd, J = 13, 10 Hz, 1H), 1.90-2.01 (m, 4H), 2.09-2.20 (m, 2H), 2.22-2.35 (m, 2H), 2.38-2.46 (m, 1H), 2.60-2.67 (m, 1H), 2.73-2.81 (m, 1H), 7.24 (d, J = 8.1 Hz, 2H), 7.50 (d, J = 8.0 Hz, 2H); APCI m/z 468.3 (M + 1) |
| 14 | 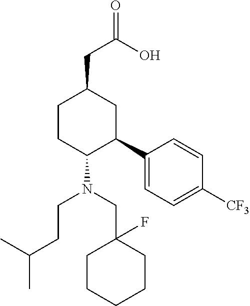 | B; P1 | 103 | {(1R,3S,4R)-4- {[(1-fluorocyclo- hexyl)methyl](3- methylbutyl) amino}-3-[4- (trifluoromethyl) phenyl]cyclohexyl} acetic acid | 0.50-0.69 (m, 1H), 0.83-0.88 (m, 6H), 0.91-1.58 (m, 15H), 1.87-2.07 (m, 4H), 2.10-2.33 (m, 4H), 2.48-2.58 (m, 1H), 2.61-2.70 (m, 1H), 2.73-2.82 (m, 1H), 2.88-2.97 (m, 1H), 7.22-7.31 (m, 2H), 7.50 (br d, J = 8 Hz, 2H); APCI m/z 484.3 (M − 1) |
| 15 | 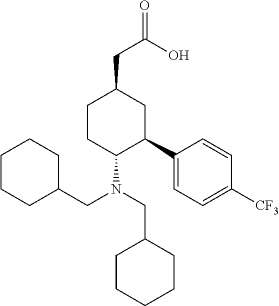 | A; P1 | 107 | {(1R,3S,4R)-4- [bis(cyclohexyl methyl)amino]- 3-[4-(trifluoro- methyl)phenyl] cyclohexyl}acetic acid | 0.33-0.43 (m, 2H), 0.67-0.78 (m, 2H), 0.89-1.29 (m, 12H), 1.37-1.53 (m, 5H), 1.57-1.69 (m, 4H), 1.88-2.02 (m, 6H), 2.10 (dd, J = 13, 4 Hz, 2H), 2.19-2.33 (m, 2H), 2.69-2.81 (m, 2H), 7.27 (d, J = 8.2 Hz, 2H), 7.49 (d, J = 8.0 Hz, 2H); 494.6 |
| 16 | 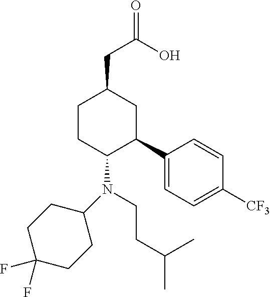 | B²; P1 | 496 | {(1R,3S,4R)-4- [(4,4-difluoro- cyclohexyl)(3- methylbutyl) amino]-3-[4- (trifluoromethyl) phenyl]cyclohexyl} acetic acid | 0.77-0.84 (m, 6H), 0.82-1.01 (m, 2H), 1.04-1.38 (m, 6H), 1.42-1.71 (m, 5H), 1.87-2.10 (m, 5H), 2.19-2.35 (m, 2H), 2.38-2.51 (m, 3H), 2.71-2.80 (m, 2H), 7.18-7.25 (m, 2H), 7.46-7.52 (m, 2H); 490.5 |
| 17 | 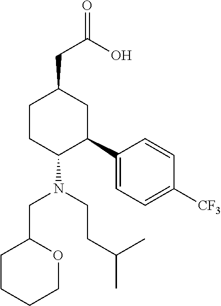 | B³; P1 | 1510 | {[1R,3S,4R)-4- [(3-methylbutyl) (tetrahydro-2H- pyran-2-ylmethyl) amino]-3-[4- (trifluoromethyl) phenyl]cyclohexyl} acetic acid | 0.66-0.77 (m, 1H), 0.81-0.85 (m, 6H), 0.91-0.99 (m, 1H), 1.04-1.20 (m, 4H), 1.28-1.46 (m, 5H), 1.53-1.61 (m, 1H), 1.87-2.05 (m, 4H), 2.11 (dd, J = 13.2, 8.2 Hz, 1H), 2.15-2.34 (m, 3H), 2.42-2.52 (m, 2H), 2.66-2.80 (m, 2H), 2.82-2.90 (m, 1H), 3.27-3.35 (m, 1H), 3.88-3.94 (m, 1H), 7.25 (d, J = 8.0 Hz, 2H), 7.51 (d, J = 8.0 Hz, 2H); 470.6 |
| 18 | 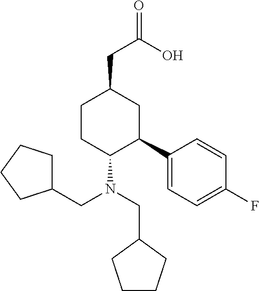 | A; P8 | 2630 | [(1R,3S,4R)-4- [bis(cyclopentyl- methyl)amino]-3- (4-fluorophenyl) cyclohexyl]acetic acid | 0.76-0.86 (m, 2H), 0.94-1.04 (m, 2H), 1.09-1.28 (m, 4H), 1.31-1.66 (m, 13H), 1.78-2.00 (m, 5H), 2.04-2.12 (m, 2H), 2.18-2.31 (m, 3H), 2.66-2.80 (m, 2H), 6.90-6.96 (m, 2H), 7.08-7.13 (m, 2H); 416.6 |
| 19 | 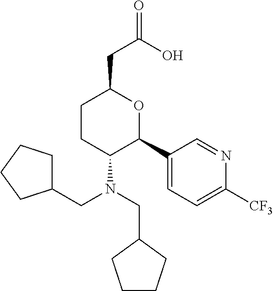 | Preparation 3, C; P2 | 837 | {(2S,5R,6S)-5- [bis(cyclopentyl- methyl)amino]-6- [6-(trifluoromethyl) pyridin-3-yl] tetrahydro-2H- pyran-2-yl}acetic acid | 0.78-0.88 (m, 2H), 0.90-1.00 (m, 2H), 1.11-1.21 (m, 2H), 1.31-1.62 (m, 9H), 1.63-1.81 (m, 3H), 1.84-1.89 (m, 1H), 1.93-2.00 (m, 1H), 2.04-2.17 (m, 3H), 2.29-2.36 (m, 2H), 2.53 (dd, half of ABX pattern, J = 16, 5 Hz, 1H), 2.62 (dd, half of ABX pattern, J = 16, 7 Hz, 1H), 2.69-2.78 (m, 1H), 3.73- 3.78 (m, 1H), 3.87-3.96 (m, 1H), 4.45 (d, J = 10 Hz, 1H), 7.64 (d, J = 8 Hz, 1H), 7.88 (d, J = 8 Hz, 1H), 8.72 (s, 1H); APCI m/z 469.4 (M + 1) |
| 20 | 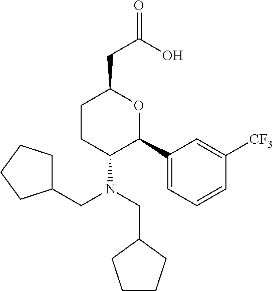 | Preparation 3, C; P2 | 959 | {(2S,5R,6S)-5- [bis(cyclopentyl- methyl)amino]-6- [3-(trifluoromethyl) phenyl]tetrahydro- 2H-pyran-2-yl} acetic acid | 0.78-0.88 (m, 2H), 0.91-1.02 (m, 2H), 1.10-1.21 (m, 2H), 1.31-1.61 (m, 11H), 1.66-1.80 (m, 3H), 1.91-1.97 (m, 1H), 2.01-2.11 (m, 3H), 2.29 (dd, J = 12.6, 5.2 Hz, 2H), 2.50 (dd, half of ABX pattern, J = 15.8, 5.7 Hz, 1H), 2.63 (dd, half of ABX pattern, J = 15.9, 7.1 Hz, 1H), 2.76-2.83 (m, 1H), 3.85- 3.92 (m, 1H), 4.38 (d, J = 10.0 Hz, 1H), 7.41 (dd, J = 7.8, 7.6 Hz, 1H), 7.50-7.56 (m, 2H), 7.63 (s, 1H); 468.7 |
| 21 | 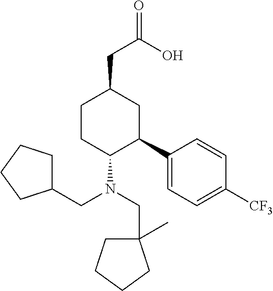 | B; P1 | 435 | {(1R,3S,4R)-4- {(cyclopentyl- methyl)[(1- methylcyclo- pentyl)methyl] amino}-3-[4- (trifluoromethyl) phenyl]cyclohexyl} acetic acid | 0.71-0.81 (m, 2H), 0.83-0.91 (m, 1H), 0.93-1.02 (m, 2H), 1.09-1.30 (m, 5H), 1.31-1.62 (m, 11H), 1.76-1.85 (m, 2H), 1.89-2.02 (m, 4H), 2.08 (dd, J = 12.5, 9.8 Hz, 2H), 2.18-2.32 (m, 4H), 2.75-2.87 (m, 2H), 7.28 (d, J = 8 Hz, 2H, assumed; partially obscured by solvent peak), 7.50 (d, J = 8.1 Hz, 2H); High-resolution MS m/z 480 (M + 1) |
| 22 | 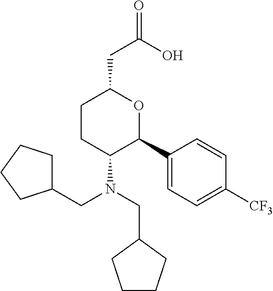 | C; P4 | 6390 | {(2R,5R,6S)-5- [bis(cyclopentyl- methyl)amino]-6- [4-(trifluoromethyl) phenyl]tetrahydro- 2H-pyran-2-yl} acetic acid | 0.81-0.91 (m, 2H), 0.94-1.04 (m, 2H), 1.15-1.24 (m, 2H), 1.32-1.63 (m, 10H), 1.69-1.93 (m, 5H), 2.00-2.15 (m, 3H), 2.32 (dd, J = 12.7, 5.1 Hz, 2H), 2.60 (dd, J = 15.0, 5.9 Hz, 1H), 2.83-2.90 (m, 1H), 3.01 (dd, J = 14.9, 8.9 Hz, 1H), 4.45-4.51 (m, 1H), 4.57 (d, J = 10.1 Hz, 1H), 7.53 (AB quartet, J_{AB} = 8.2 Hz, Δν_{AB} = 26.9 Hz, 4H); 468.5 |
| 23 | 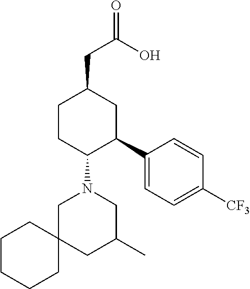 | A^{5.6}; P1 | 868 | {(1R,3S,4R)-4- (4-methyl-2- azaspiro[5.5]undec- 2-yl)-3-[4- (trifluoromethyl) phenyl]cyclohexyl} acetic acid | 0.36-0.44 (m, 1H), 0.71 (d, J = 6.4 Hz) and 0.67 (d, J = 6.2 Hz, 3H total), 0.75-1.60 (m, 16H), 1.88-2.04 (m, 5H), 2.13- 2.30 (m, 2H), 2.42-2.61 (m, 2H), 2.66-2.88 (m, 2H), 7.22- 7.29 (m, 2H), 7.49 (d, J = 8.0 Hz, 2H); 452.6 |
| 24 | 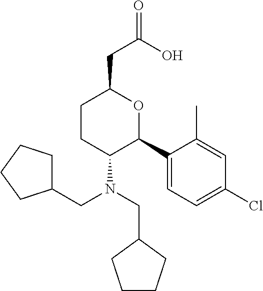 | Preparation 3, C; P2 | 9190 | [(2R,5R,6S)-5- [bis(cyclopentyl- methyl)amino]-6- (4-chloro-2- methylphenyl) tetrahydro-2H- pyran-2-yl]acetic acid | 0.77-0.87 (m, 2H), 0.96-1.06 (m, 2H), 1.29-1.74 (m, 13H), 1.83-1.95 (m, 3H), 2.04-2.17 (m, 4H), 2.29 (dd, J = 12.7, 5.8 Hz, 2H), 2.37 (s, 3H), 2.52 (dd, half of ABX pattern, J = 16.1, 4.9 Hz, 1H), 2.59 (dd, half of ABX pattern, J = 16.1, 7.8 Hz, 1H), 3.08-3.15 (m, 1H), 3.86- 3.93 (m, 1H), 4.56 (d, J = 10.0 Hz, 1H), 7.12-7.16 (m, 2H), 7.32 (d, J = 9.0 Hz, 1H); LCMS m/z 446.5, 448.5 (M − 1) |
| 25 | 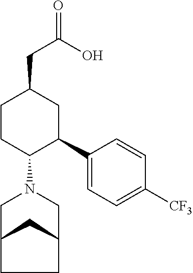 | A⁷; P1 | 8840 | {(1R,3S,4R)-4- [(1R,5S)-3- azabicyclo[3.2.1] oct-3-yl]-3-[4- (trifluoromethyl) phenyl]cyclohexyl} acetic acid | 0.46-0.54 (m, 1H), 0.83-0.92 (m, 1H), 1.02-1.45 (m, 7H), 1.84-2.00 (m, 6H), 2.16-2.35 (m, 5H), 2.57-2.65 (m, 2H), 2.74-2.82 (m, 1H), 7.27 (d, J = 8 Hz, 2H, assumed; partially obscured by solvent peak), 7.53 (d, J = 8.1 Hz, 2H); 3.96.1 |
| 26 | 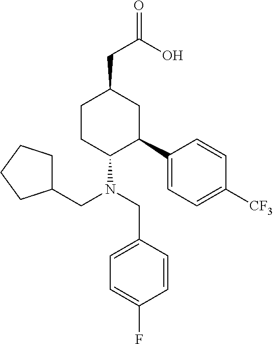 | B; P1 | 190 | {(1R,3S,4R)-4- [(cyclopentyl- methyl)(4- fluorobenzyl) amino]-3-[4- (trifluoromethyl) phenyl]cyclohexyl} acetic acid | 0.63-0.72 (m, 1H), 0.90-1.00 (m, 1H), 1.07-1.49 (m, 8H), 1.53-1.63 (m, 1H), 1.80-2.03 (m, 4H), 2.04-2.13 (m, 2H), 2.18-2.32 (m, 2H), 2.36 (dd, J = 12.5, 5.0 Hz, 1H), 2.78-2.89 (m, 2H), 3.22 (d, J = 13.8 Hz, 1H), 3.66 (d, J = 13.9 Hz, 1H), 6.76-6.86 (m, 4H), 7.13 (d, J = 8.0 Hz, 2H), 7.49 (d, J = 8.0 Hz, 2H); APCI m/z 492.1 (M + 1) |
| 27 | 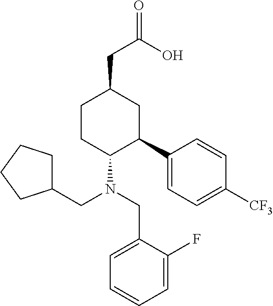 | B; P1 | 155⁸ | {(1R,3S,4R)-4- [(cyclopentyl- methyl)(2- fluorobenzyl) amino]-3-[4- (trifluoromethyl) phenyl]cyclohexyl} acetic acid | 0.66-0.75 (m, 1H), 0.93-1.02 (m, 1H), 1.11-1.76 (m, 9H), 1.81-2.33 (m, 8H), 2.40 (dd, J = 12, 5 Hz, 1H), 2.78-2.87 (m, 2H), 3.56 (s, 2H), 6.67-6.73 (m, 1H), 6.84-6.90 (m, 1H), 6.91-6.97 (m, 1H), 7.06 (d, J = 8 Hz, 2H), 7.11-7.18 (m, 1H), 7.43 (d, J = 8 Hz, 2H); APCI m/z 492.0 (M + 1) |
| 28 | 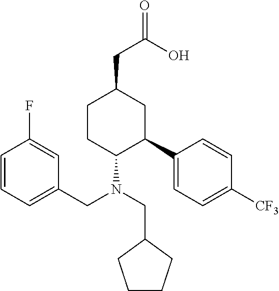 | B; P1 | 230⁸ | {(1R,3S,4R)-4- [(cyclopentyl- methyl)(3- fluorobenzyl) amino]-3-[4- (trifluoromethyl) phenyl]cyclohexyl} acetic acid, hydro- chloride salt | 0.67-0.76 (m, 1H), 0.92-1.02 (m, 1H), 1.07-1.50 (m, 8H), 1.54-1.63 (m, 1H), 1.80-2.03 (m, 4H), 2.07-2.15 (m, 2H), 2.19-2.32 (m, 2H), 2.38 (dd, J = 12.5, 5.1 Hz, 1H), 2.78-2.89 (m, 2H), 3.27 (d, J = 14.3 Hz, 1H), 3.68 (d, J = 14.4 Hz, 1H), 6.49-6.54 (m, 1H), 6.65 (br d, J = 8 Hz, 1H), 6.82-6.88 (m, 1H), 7.07-7.13 (m, 1H), 7.14 (br d, J = 8 Hz, 2H), 7.51 (d, J = 8 Hz, 2H)⁴; 492.5 |
| 29 | 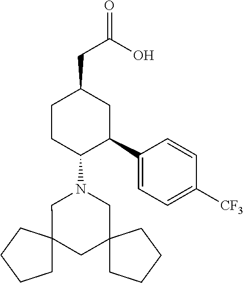 | A⁹; P1 | 193⁸ | {(1R,3S,4R)-4- (13-azadispiro [4.1.4.3]tetradec- 13-yl)-3-[4- (trifluoromethyl) phenyl]cyclohexyl} acetic acid | 0.88-1.05 (m, 6H), 1.07-1.22 (m, 6H), 1.31-1.49 (m, 9H), 1.85-2.00 (m, 6H), 2.14-2.27 (m, 4H), 2.68-2.82 (m, 2H), 7.26 (d, J = 8.2 Hz, 2H), 7.51 (d, J = 8.0 Hz, 2H); 478.3 |

¹Diastereomers of the final product were removed via chromatography on a Chiralcel OD-H column, 5 μm (Mobile phase: 90/10 CO₂/propanol).
²The reductive amination with the ketone was carried out by initial formation of the imine in benzene at reflux, using molecular sieves. The
sodium borohydride reaction was carried out at 0° C.
³Potassium carbonate-mediated alkylation with 2-(bromomethyl)tetrahydro-2H-pyran was used to introduce one substituent.
⁴Characterization data was obtained on the neutral compound, prior to salt formation.
⁵The requisite dialdehyde can be obtained from the corresponding diester or ester-acid via reduction with lithium aluminum hydride followed by
Swern oxidation. The diester or ester-acid can be prepared using methods described by D. Hepworth et al., PCT International Application
WO 2006/027680, March 16, 2006.
⁶This compound is a mixture of diastereomers at the methyl group.
⁷The requisite dialdehyde can be prepared by the method of P. C. Gareiss et al., Eur. J. Org. Chem. 2007, 53-61.
⁸IC₅₀ value is from a single determination.
⁹Cyclopentanecarboxylic acid was deprotonated with lithium diisopropylamide, then reacted with diiodomethane to generate 1,1′-
methylenedicyclopentanecarboxylic acid. Lithium aluminum hydride reduction to the diol was followed by Swern oxidation to provide the
requisite dialdehyde.

TABLE 3

| | | | | | HPLC |
|---|---|---|---|---|---|
| | | Method | | | retention |
| | | of | | | time |
| | | preparation⁸; | Aβ | | (minutes); |
| | | starting | 42B | | Mass |
| | | material | IC₅₀ | | spectrum |
| Ex # | Structure | (s) | (nM)⁹ | IUPAC Name | m/z (M + 1) |
| 30 | 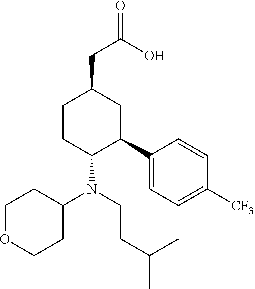 | B¹; P1 | 7310 | {(1R,3S,4R)-4-[(3- methylbutyl)(tetrahydro-2H- pyran-4-yl)amino]-3-[4- (trifluoromethyl)phenyl] cyclohexyl}acetic acid, ammonium salt | 2.10⁴; 456.2 |
| 31 | 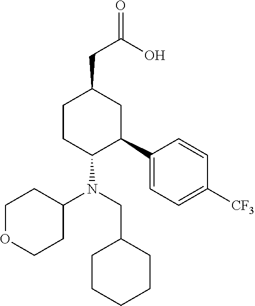 | B¹; P1 | 1300 | {(1R,3S,4R)-4- [(cyclohexylmethyl)(tetrahydro- 2H-pyran-4-yl)amino]-3-[4- (trifluoromethyl)phenyl] cyclohexyl}acetic acid, ammonium salt | 2.26⁴; 482.3 |
| 32 | 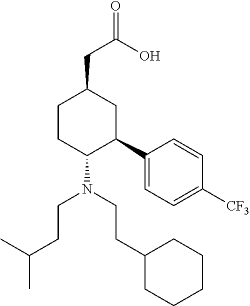 | B; P1 | 598 | {(1R,3S,4R)-4-[(2- cyclohexylethyl)(3- methylbutyl)amino]-3-[4- (trifluoromethyl)phenyl] cyclohexyl}acetic acid, trifluoroacetate salt | 3.23⁵; 482.2 |
| 33 | 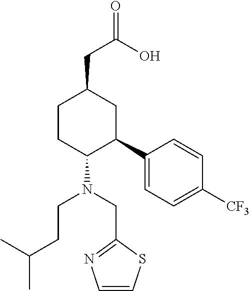 | B; P1 | 3020 | {(1R,3S,4R)-4-[(3- methylbutyl)(1,3-thiazol-2- ylmethyl)amino]-3-[4- (trifluoromethyl)phenyl] cyclohexyl}acetic acid, trifluoroacetate acid | 2.84⁵; 469.1 |
| 34 | 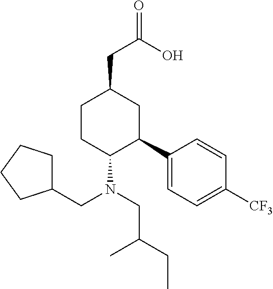 | B; P1 | 359¹⁰ | {(1R,3S,4R)-4- [(cyclopentylmethyl)(2- methylbutyl)amino]-3-[4- (trifluoromethyl)phenyl] cyclohexyl}acetic acid, ammonium salt | 2.89⁵; 454.3 |
| 35 | 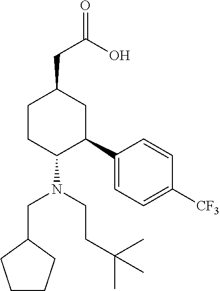 | B; P1 | 516 | {(1R,3S,4R)-4- [(cyclopentylmethyl)(3,3- dimethylbutyl)amino]-3-[4- (trifluoromethyl)phenyl] cyclohexyl}acetic acid, ammonium salt | 3.02⁵; 468.3 |
| 36 | 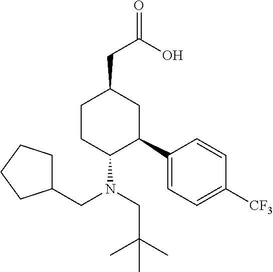 | B; P1 | 349 | {(1R,3S,4R)-4- [(cyclopentylmethyl)(2,2- dimethylpropyl)amino]-3-[4- (trifluoromethyl)phenyl] cyclohexyl}acetic acid, ammonium salt | 2.85 and 2.93⁵; 454.3 and 454.3 |
| 37 | 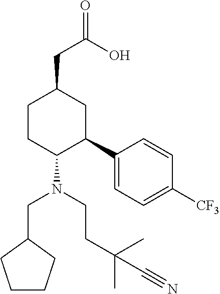 | B²; P1 | 2170 | {(1R,3S,4R)-4-[(3-cyano-3- methylbutyl)(cyclopentylmethyl) amino]-3-[4-(trifluoromethyl) phenyl]cyclohexyl}acetic acid, trifluoroacetate salt | 2.77⁵; 479.3 |
| 38 | 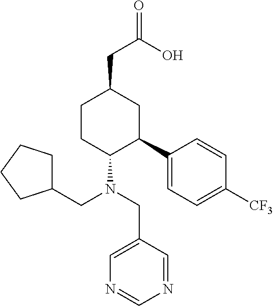 | B; P1 | 5220 | {(1R,3S,4R)-4- [(cyclopentylmethyl)(pyrimidin- 5-ylmethyl)amino]-3-[4- (trifluoromethyl)phenyl] cyclohexyl}acetic acid, trifluoroacetate salt | 2.87⁵; 476.3 |
| 39 | 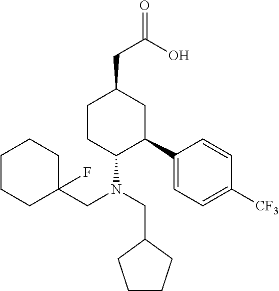 | B; P1 | 109 | {(1R,3S,4R)-4- {(cyclopentylmethyl)[(1- fluorocyclohexyl)methyl] amino}-3-[4- (trifluoromethyl)phenyl] cyclohexyl}acetic acid, ammonium salt | 3.17⁵; 498.2 |
| 40 | 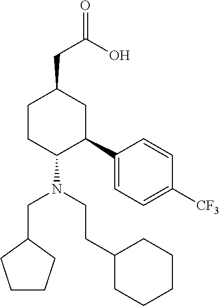 | B; P1 | 621 | {(1R,3S,4R)-4-[(2- cyclohexylethyl)(cyclopentyl- methyl)amino]-3-[4- (trifluoromethyl)phenyl] cyclohexyl}acetic acid, trifluoroacetate salt | 3.23⁵; 494.3 |
| 41 | 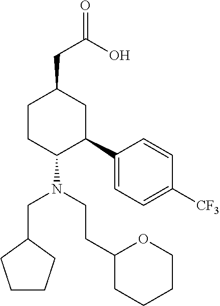 | B³; P1 | 5630 | {(1R,3S,4R)-4- {(cyclopentylmethyl)[2- (tetrahydro-2H-pyran-2- yl)ethyl]amino}-3-[4- (trifluoromethyl)phenyl] cyclohexyl}acetic acid, trifluoroacetate salt | 3.00⁵; 496.3 |
| 42 | 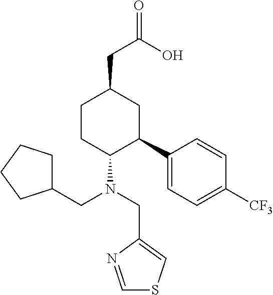 | B; P1 | 1970 | {(1R,3S,4R)-4- [(cyclopentylmethyl)(1,3- thiazol-4-ylmethyl)amino]-3-[4- (trifluoromethyl)phenyl] cyclohexyl}acetic acid, trifluoroacetate salt | 2.74⁵; 481.2 |
| 43 | 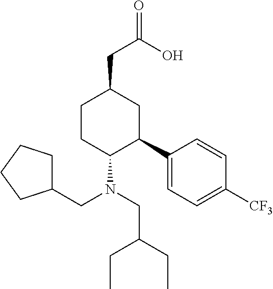 | B; P1 | 162¹⁰ | {(1R,3S,4R)-4- [(cyclopentylmethyl)(2- ethylbutyl)amino]-3-[4- (trifluoromethyl)phenyl] cyclohexyl}acetic acid, trifluoroacetate salt | 3.04⁵; 468.3 |
| 44 | 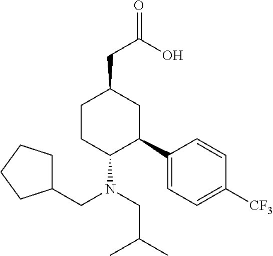 | B; P1 | 318 | {(1R,3S,4R)-4- [(cyclopentylmethyl)(isobutyl) amino]-3-[4- (trifluoromethyl)phenyl] cyclohexyl}acetic acid, trifluoroacetic salt | 2.80⁵; 440.3 |
| 45 | 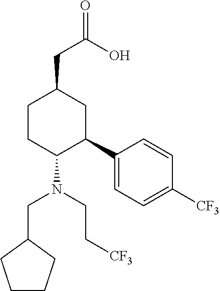 | B; P1 | 484 | {(1R,3S,4R)-4- [(cyclopentylmethyl)(3,3,3- trifluoropropyl)amino]-3-[4- (trifluoromethyl)phenyl] cyclohexyl}acetic acid, trifluoroacetate salt | 2.91⁵; 480.2 |
| 46 | 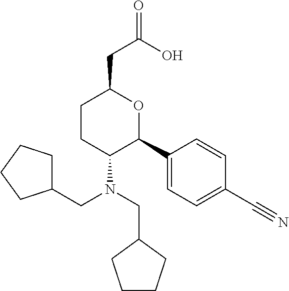 | Preparation 3, C; P3 | 2550 | [(2S,5R,6S)-5- [bis(cyclopentylmethyl)amino]- 6-(4-cyanophenyl)tetrahydro- 2H-pyran-2-yl]acetic acid, ammonium salt | 2.57⁵; 425.3 |
| 47 | 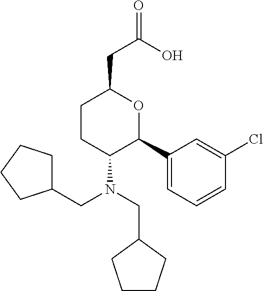 | Preparation 3, C; P3 | 1840 | [(2S,5R,6S)-5- [bis(cyclopentylmethyl)amino]- 6-(3-chlorophenyl)tetrahydro- 2H-pyran-2-yl]acetic acid, ammonium salt | 2.77⁵; 434.2, 436.2 |
| 48 | 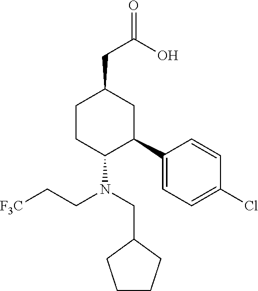 | B; P7 | 441¹⁰ | {(1R,3S,4R)-3-(4- chlorophenyl)-4- [(cyclopentylmethyl)(3,3,3- trifluoropropyl)amino] cyclohexyl}acetic acid, trifluoroacetate salt | 2.65⁶; 446.2, 448.2 |
| 49 | 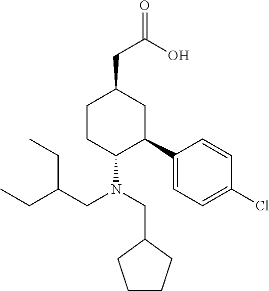 | B; P7 | 477 | {(1R,3S,4R)-3-(4- chlorophenyl)-4- [(cyclopentylmethyl)(2- ethylbutyl)amino]cyclohexyl} acetic acid, ammonium salt | 2.90⁶; 434.3, 436.3 |
| 50 | 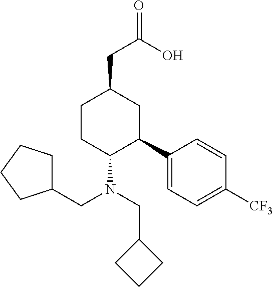 | B³; P1 | 463¹⁰ | {(1R,3S,4R)-4- [(cyclobutylmethyl) (cyclopentyl)methyl)amino]- 3-[4-(trifluoromethyl)phenyl] cyclohexyl}acetic acid, ammonium salt | 2.86⁵; 452.3 |
| 51 | 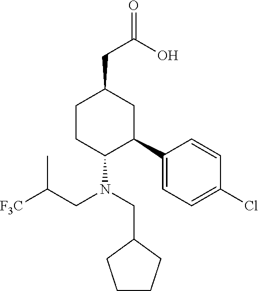 | B; P7 | 382 | {(1R,3S,4R)-3-(4- chlorophenyl)-4- [(cyclopentylmethyl)(3,3,3- trifluoro-2-methylpropyl) amino]cyclohexyl}acetic acid, trifluoroacetate salt | 3.64⁵; 460.1, 462.1 |
| 52 | 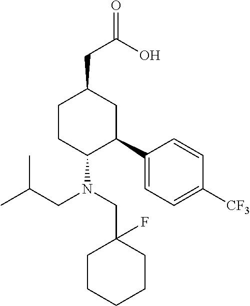 | B; P1 | 190 | {(1R,3S,4R)-4-{[(1- fluorocyclohexyl)methyl] (isobutyl)amino}-3-[4- (trifluoromethyl)phenyl] cyclohexyl}acetic acid, trifluoroacetate salt | 3.04⁵; 472.2 |
| 53 | 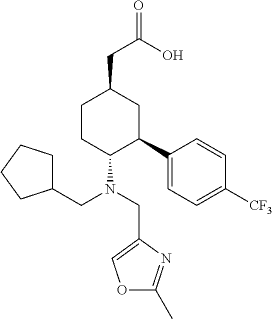 | B; P1 | 959 | {(1R,3S,4R)-4- {(cyclopentylmethyl)[(2- methyl-1,3-oxazol-4- yl)methyl]amino}-3-[4- (trifluoromethyl)phenyl] cyclohexyl}acetic acid, ammonium salt | 2.84⁵; 479.1 |
| 54 | 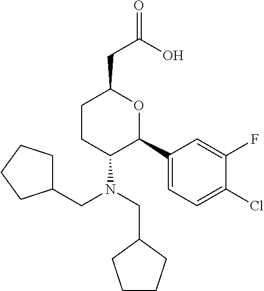 | Preparation 3, C; P3 | 1050 | [(2S,5R,6S)-5- [bis(cyclopentylmethyl)amino]- 6-(4-chloro-3- fluorophenyl)tetrahydro-2H- pyran-2-yl]acetic acid, ammonium acid | 3.06⁷; 451.9, 453.9 |
| 55 | 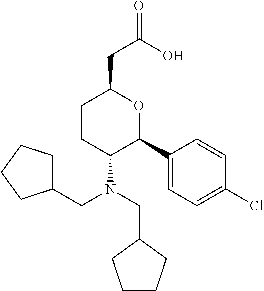 | Preparation 3, C; P2 | 892 | [(2S,5R,6S)-5- [bis(cyclopentylmethyl)amino]- 6-(4-chlorophenyl)tetrahydro- 2H-pyran-2-yl]acetic acid | 9.86¹¹; 434.3, 436.3 |
| 56 | 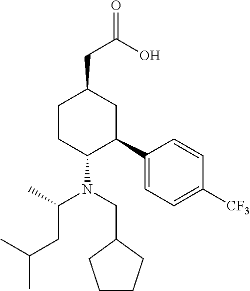 | B¹²; P1 | 544 | {(1R,3S,4R)-4- {(cyclopentylmethyl)[(1S)-1,3- dimethylbutyl]amino}-3-[4- (trifluoromethyl)phenyl] cyclohexyl}acetic acid | 2.17¹³; 468.7 |
| 57 | 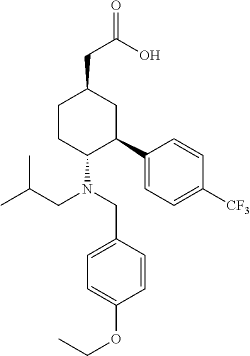 | F | 171¹⁴ | {(1R,3S,4R)-4-[(4- ethoxybenzyl)(isobutyl)amino]- 3-[4-(trifluoromethyl)phenyl] cyclohexyl}acetic acid, trifluoroacetate salt | 2.84¹⁵; 492 |
| 58 | 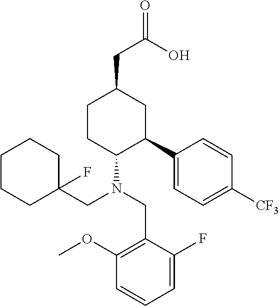 | F | 309¹⁴ | {(1R,3S,4R)-4-{[(1- fluorocyclohexyl)methyl](2- fluoro-6-methoxybenzyl) amino}-3-[4-(trifluoromethyl) phenyl]cyclohexyl}acetic acid, trifluoroacetate salt | 2.34¹⁶; 554 |
| 59 | 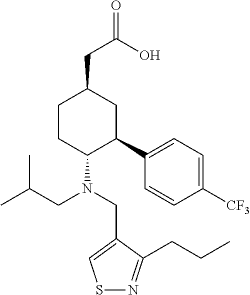 | F | 349¹⁴ | {(1R,3S,4R)-4-{isobutyl[(3- propylisothiazol-4-yl)methyl] amino}-3-[4-(trifluoromethyl) phenyl]cyclohexyl}acetic acid, trifluoroacetate salt | 3.04¹⁵; 497 |
| 60 | 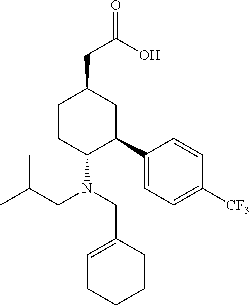 | F | 154¹⁴ | {(1R,3S,4R)-4-[(cyclohex-1- en-1-ylmethyl)(isobutyl)amino]- 3-[4-(trifluoromethyl)phenyl] cyclohexyl}acetic acid, trifluoroacetate salt | 2.81¹⁵; 452 |
| 61 | 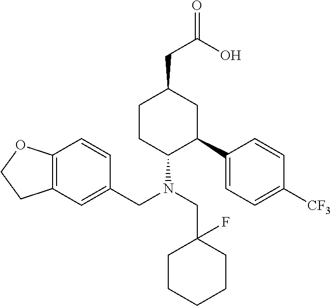 | F | 607¹⁴ | {(1R,3S,4R)-4-{(2,3-dihydro-1- benzofuran-5-ylmethyl)[(1- fluorocyclohexyl)methyl] amino}-3-[4- (trifluoromethyl)phenyl] cyclohexyl}acetic acid, trifluoroacetate salt | 2.56¹⁶; 548 |
| 62 | 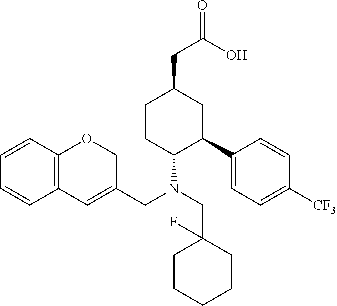 | F | 223¹⁴ | {(1R,3S,4R)-4-{(2H-chromen- 3-ylmethyl)[(1- fluorocyclohexyl)methyl] amino}-3-[4-(trifluoromethyl) phenyl]cyclohexyl}acetic acid, trifluoroacetate salt | 2.94¹⁷; 560 |
| 63 | 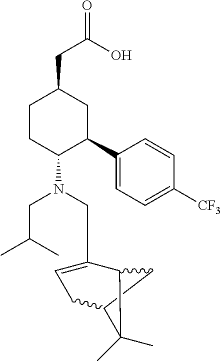 | F | 336¹⁴ | {(1R,3S,4R)-4-{[(6,6- dimethylbicyclo[3.1.1]hept-2- en-2-yl)methyl](isobutyl) amino}-3-[4- (trifluoromethyl)phenyl] cyclohexyl}acetic acid, trifluoroacetate salt | 2.46¹⁶; 492 |
| 64 | 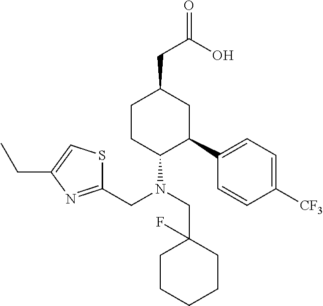 | F | 840¹⁴ | {(1R,3S,4R)-4-{[(4-ethyl-1,3- thiazol-2-yl)methyl][(1- fluorocyclohexyl)methyl] amino}-3-[4- (trifluoromethyl)phenyl] cyclohexyl}acetic acid, trifluoroacetate salt | 3.10¹⁶; 541 |
| 65 | 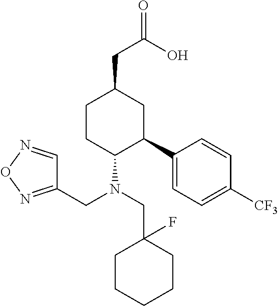 | F | 509¹⁴ | {(1R,3S,4R)-4-{(2,1,3- benzoxadiazol-5-ylmethyl)[(1- fluorocyclohexyl)methyl] amino}-3-[4-(trifluoromethyl) phenyl]cyclohexyl}acetic acid, trifluoroacetate salt | 2.77¹⁷; 548 |
| 66 | 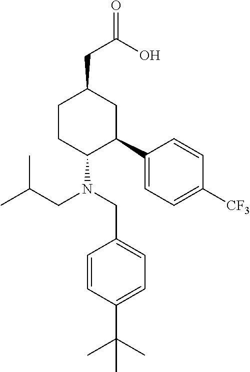 | F | 268¹⁴ | {(1R,3S,4R)-4-[(4-tert- butylbenzyl)(isobutyl)amino]- 3-[4-(trifluoromethyl)phenyl] cyclohexyl}acetic acid, trifluoroacetate salt | 2.53¹⁶; 504 |
| 67 | 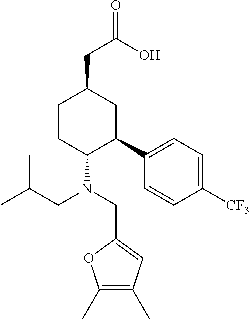 | F | 411¹⁴ | {(1R,3S,4R)-4-{[(4,5-dimethyl- 2-furyl)methyl](isobutyl) amino}-3-[4-(trifluoromethyl) phenyl]cyclohexyl}acetic acid, trifluoroacetate salt | 2.82¹⁵; 466 |

¹Reductive aminations with ketones were carried using trimethyl orthoformate as solvent.
²Reaction of 2-methylpropanenitrile and 2-bromo-1,1-dimethoxyethane with sodium amide provided 4,4-dimethyl-2,2-dimethylbutanenitrile.
Formic acid-mediated acetal hydrolysis afforded the requisite aldehyde.
³A Swern oxidation of the corresponding alcohol provided the requisite aldehyde.
⁴HPLC conditions. Column: Waters XBridge C₁₈, 4.6 × 50 mm, 5 μm; Mobile phase A: 0.03% NH₄OH in water (v/v); Mobile phase B: 0.03%
NH₄OH in acetonitrile (v/v); Gradient: 15% to 95% B over 4.0 minutes (linear gradient); Flow rate: 2.0 mL/min.
⁵HPLC conditions. Column: Waters Atlantis dC₁₈, 4.6 × 50 mm, 5 μm; Mobile phase A: 0.05% TFA in water (v/v); Mobile phase B: 0.05% TFA
in acetonitrile (v/v); Gradient: 5% to 95% B over 4.0 minutes (linear gradient); Flow rate: 2.0 mL/min.
⁶HPLC conditions were the same as those described in footnote 5 except that the gradient was run from 10% to 95% B.
⁷HPLC conditions. Column: Waters Sunfire C₁₈, 4.6 × 50 mm, 5 μm; Mobile phase A: 0.05% TFA in water (v/v); Mobile phase B: 0.05% TFA in
acetonitrile (v/v); Gradient: 5% to 95% B over 4.0 minutes (linear gradient); Flow rate: 2.0 mL/min.
⁸Purification of compounds in this table was carried out by reversed-phase HPLC using one of the following systems: 1) Column: Waters XBridge
C₁₈, 5 μm; Mobile phase A: 0.03% NH₄OH in water (v/v); Mobile phase B: 0.03% NH₄OH in acetonitrile (v/v); Gradient:
10% to 100% B. 2) Column: Waters Sunfire C₁₈, 5 μm; Mobile phase A: 0.05% TFA in water (v/v), Mobile phase B: 0.05% TFA in acetonitrile
(v/v); Gradient: 20% to 100% B.
⁹IC₅₀ values represent the geometric mean of 2-4 determinations, unless otherwise indicated.
¹⁰IC₅₀ values represent the geometric mean of 6-8 determinations.
¹¹HPLC conditions. Column: XBridge C₁₈, 4.6 × 50 mm, 5 μm; Mobile phase A: 0.1% TFA in water (v/v); Mobile phase B: 0.1% TFA in acetonitrile
(v/v); Gradient: 0-1.5 min, 5% B; 1.5-10 min, 5% to 100% B; 10-11 min, 100% B; Flow rate: 15 mL/min.
¹²Diastereomers of the final product separated via chromatography on a Chiralcel OJ-H column, 5 μm (Mobile phase: 95/5 CO₂/MeOH with 0.2%
isopropylamine modifier); this compound was the later-eluting isomer. The stereochemistry of the methyl group was arbitrarily assigned.
¹³HPLC conditions. Column: Chiralcel OJ-H, 4.6 × 25 cm, 5 μm; Mobile phase: 95/5 CO₂/MeOH with 0.2% isopropylamine modifier; Flow rate: 2.5 mL/min.
¹⁴IC₅₀ value is from a single determination.
¹⁵HPLC conditions. Column: Welch XB-C18, 2.1 × 50 mm, 5 μm; Mobile phase A: 0.0375% TFA in water (v/v); Mobile phase B: 0.01875% TFA in
acetonitrile (v/v). Gradient: 0-0.5 min, 10% B; 0.50-4.00 min, 10% to 100% B. Flow rate: 0.8 mL/min.
¹⁶HPLC conditions: identical to footnote 15 except that Gradient: 0-0.5 min, 25% B; 0.50-3.50 min, 25% to 100% B.
¹⁷HPLC conditions: identical to footnote 15 except that Gradient: 0-0.4 min, 40% B; 0.40-2.80 min, 40% to 100% B, 2.80-4.00 min, 100% B.

### Cell-Based γ-Secretase Assay with ELISA Readout

The ability of compounds to modulate production of amyloid beta protein A6(1-42) was determined using human WT-APP overexpressing CHO cells. Cells were plated at 22,000 cells/100 μL well in 96 well tissue culture treated, clear plates (Falcon) in DMEM/F12 based medium and incubated for 24 hours at 37° C. Compounds for testing were diluted in 100% DMSO to achieve an eleven points, half log, dose response for IC₅₀ determinations. Compounds were added in fresh medium to achieve 1% final DMSO. Appropriate vehicle and inhibitor controls were added to obtain maximum and minimum inhibition values for the assay before the plates were incubated for about 24 hours at 37° C.

Coating of ELISA assay plates was initiated by addition of 50 μL/well of an in-house Aβ(1-42) specific antibody at (4 μg/mL) in 0.1 M NaHCO₃ (pH 9.0) into black 384-well Maxisorp® plates (Nunc) and incubated overnight at 4° C. The capture antibody was then aspirated from the ELISA assay plates and 100 μL/well of Blocking Buffer (Dulbecco's PBS, 1.5% BSA (Sigma A7030)) added. Ambient temperature incubation was allowed to proceed for a minimum of two hours before washing 2×100 μL with Wash Buffer (Dulbecco's PBS, 0.05% Tween 20). Assay Buffer (Dulbecco's PBS, 1.0% BSA (Sigma A7030), 0.05% Tween 20) 20 μL/well was then added.

After incubation overnight at 37° C., 5% CO₂, 40 μL (in duplicate) of experimental conditioned media were transferred into wells of the blocked ELISA plates containing the capture antibody, followed by overnight incubation at 4° C.

Cell toxicity was measured in the corresponding cells after removal of the media for the Aβ(1-42) assay by a colorimetric cell proliferation assay (CellTiter 96® AQᵤₑₒᵤₛ One Solution Cell Proliferation Assay, Promega) according to the manufacturer's instructions.

After overnight incubation of the ELISA assay plates at 4° C., unbound Aβ peptides were removed thorough (4×100 μL) washes with Wash Buffer. Europium (Eu) labeled (custom labeled, Perkin Elmer) Aβ(1-16) 6e10 Monoclonal Antibody (Covance #SIG-39320 was added, (50 μL/well Eu-6e10 @ 1:5000, 20 uM EDTA) in Assay Buffer. Incubation at ambient temperature for a minimum of 2 hours was followed by (4×100 μL) washes with Wash Buffer, before 50 μL/well of Delfia Enhancement Solution (PerkinElmer) was added. Following a one hour ambient temperature incubation, the plates were read on an EnVision plate reader (PerkinElmer) using standard DELFIA TRF settings. Data analysis including inhibitory IC₅₀ determination was performed using nonlinear regression fit analysis (in-house software) and the appropriate plate mean values for the maximum and minimum inhibition controls.

1. A compound having the structure of formula I: 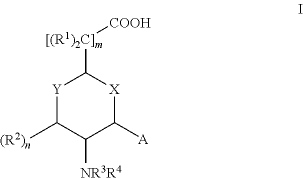
wherein A is C₆₋₁₀aryl or 5- to 10-membered heteroaryl, optionally substituted with one to three R⁷;
X and Y are independently C(R⁹)₂, NR¹⁰ or O, wherein at least one of X or Y is C(R⁹)₂;
each R¹ is independently hydrogen, C₁₋₆alkyl or —(CH₂)ₜ—C₃₋₇cycloalkyl; or two R¹ substituents together with the carbon to which they are bonded can form a C₃₋₇cycloalkyl;
each R² is independently CF₃, fluorine, C₁₋₃alkyl, C₃₋₇cycloalkyl, or OR⁵, or two R² substituents together with the carbon to which they are bonded can form a C₃₋₄cycloalkyl;
R³ and R⁴ are independently C₁₋₆alkyl, C₃₋₇cycloalkyl, C₆₋₁₀aryl, 5- to 10-membered heteroaryl, or 4- to 10-membered heterocycloalkyl, wherein said alkyl, cycloalkyl, aryl, heteroaryl and heterocycloalkyl are optionally substituted by C₁₋₆alkyl, halogen, oxo, cyano, —CF₃, C₃₋₇cycloalkyl, C₆₋₁₀aryl, 5- to 10-membered heteroaryl, or 4- to 10-membered heterocycloalkyl, wherein said cycloalkyl, aryl, heterocycloalkyl, or heteroaryl substituents can be further substituted with one to three C₁₋₆alkyl, halogen, —(CH₂)ₜ—CF₃, hydroxy, oxo, or cyano;
alternatively, R³ and R⁴ together with the nitrogen to which they are bonded form a 4- to 10-membered heterocycloalkyl or 5- to 10-membered heteroaryl wherein said heterocycloalkyl or heteroaryl is optionally substituted with one to six R⁶ wherein two R⁶ together with the atom or atoms to which they are bonded, optionally including additional atoms of the heterocycloalkyl in the case of a bridged system, can form a C₃₋₇cycloalkyl, C₆₋₁₀aryl, 5- to 10-membered heteroaryl, or 4- to 10-membered heterocycloalkyl, wherein said cycloalkyl, aryl, heterocycloalkyl, or heteroaryl can be further substituted with one to three C₁₋₆alkyl, halogen, —CF₃, hydroxy, oxo, or cyano, and said C₁₋₆alkyl is optionally further substituted with one to three fluorine or —(CH₂)ₜ—CF₃;
each R⁵ is independently hydrogen or C₁₋₃alkyl, wherein said alkyl can be substituted with one to three fluorines;
each R⁶ is independently C₁₋₆alkyl, fluorine, —(CH₂)ₜ—CF₃, hydroxy, oxo, or cyano;
each R⁷ is independently —(CH₂)ₜ—CF₃, cyano, halogen, C₁₋₃alkyl, C₃₋₇cycloalkyl, or —OR⁸;
each R⁸ is independently hydrogen, C₁₋₃alkyl or —(CH₂)ₜ—CF₃;
each R⁹ is independently hydrogen, CF₃, fluorine, C₁₋₃alkyl, C₃₋₇cycloalkyl or OR⁵, or two R⁹ substituents together with the carbon to which they are bonded can form a C₃₋₄cycloalkyl;
R¹⁰ is hydrogen, —(CH₂)ₜ—CF₃, C₁₋₃alkyl or C₃₋₇cycloalkyl;
each n is an integer independently selected from 0, 1, or 2;
each m is an integer independently selected from 0, 1, or 2; and
each t is an integer independently selected from 0, 1, or 2; or pharmaceutically acceptable salt thereof.

2. A compound according to claim 1 wherein Y is C(R⁹)₂ wherein each R⁹ is hydrogen, and X is O; or pharmaceutically acceptable salt thereof.

3. A compound according to claim 1 wherein X and Y are C(R⁹)₂ wherein each R⁹ is hydrogen; or pharmaceutically acceptable salt thereof.

4. A compound of formula Ia, according to claim 2, having the structure: 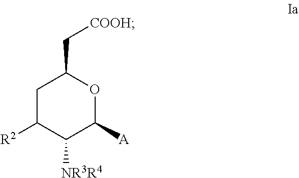
or pharmaceutically acceptable salt thereof.

5. A compound of formula Ib, according to claim 3, having the structure: 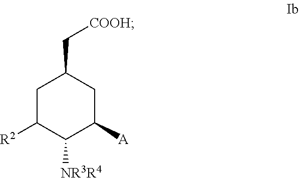
or pharmaceutically acceptable salt thereof.

6. A compound as in claims 4 or 5 wherein A is C₆₋₁₀aryl substituted with one R⁷; and R⁷ is —(CH₂)ₜ—CF₃, cyano, halogen, C₁₋₃alkyl, C₃₋₇cycloalkyl, or —OR⁸; or pharmaceutically acceptable salt thereof.

7. A compound as in claim 6, wherein A is phenyl; and R⁷ is —(CH₂)ₜ—CF₃; or pharmaceutically acceptable salt thereof.

8. A compound as in claim 7, wherein R³ and R⁴ are both C₁₋₆alkyl, optionally substituted by fluorine, oxo, cyano, —CF₃, C₃₋₇cycloalkyl, C₆₋₁₀aryl, 5- to 10-membered heteroaryl, or 4- to 10-membered heterocycloalkyl, wherein said cycloalkyl, aryl, heterocycloalkyl, or heteroaryl substituents can be further substituted with one to three C₁₋₆alkyl, halogen, —(CH₂)ₜ—CF₃, hydroxy, oxo, or cyano; and R² is hydrogen; or pharmaceutically acceptable salt thereof.

9. A compound as in claim 7, wherein R³ and R⁴ are independently C₁₋₆alkyl, and both R³ and R⁴ are substituted by C₃₋₇cycloalkyl wherein said cycloalkyls are optionally independently substituted with one to three C₁₋₆alkyl, fluorine, —(CH₂)ₜ—CF₃, hydroxy, oxo, or cyano; and R² is hydrogen; or pharmaceutically acceptable salt thereof.

10. A compound as in claim 7, wherein R³ and R⁴ are independently C₁₋₆alkyl, and R³ is substituted by C₆₋₁₀aryl and R⁴ is substituted by C₃₋₇cycloalkyl wherein said aryl and cycloalkyl are optionally independently substituted with one to three C₁₋₆alkyl, halogen, —(CH₂)ₜ—CF₃, hydroxy, oxo, or cyano; and R² is hydrogen; or pharmaceutically acceptable salt thereof.

11. A compound as in claim 7, wherein R³ and R⁴ are independently C₁₋₆alkyl, and R³ is substituted by 5- to 10-membered heteroaryl and R⁴ is substituted by C₃₋₆cycloalkyl wherein said heteroaryl or cycloalkyl are optionally independently substituted with one to three C₁₋₆alkyl, halogen, —(CH₂)ₜ—CF₃, hydroxy, oxo, or cyano; and R² is hydrogen; or pharmaceutically acceptable salt thereof.

12. A compound as in claim 7, wherein R³ and R⁴ together with the nitrogen to which they are bonded form a 4- to 10-membered heterocycloalkyl, optionally substituted with one to six R⁶; and R² is hydrogen; or pharmaceutically acceptable salt thereof.

13. (canceled)

14. A compound which is:
{(1R,3S,4R)/(1S,3R,4S)-4-[(3-methylbutyl)(3,3,3-trifluoropropyl)amino]-3-[4-(trifluoromethyl)phenyl]cyclohexyl}acetic acid, hydrochloride salt;
2{(2R,4S,5S)-5-[(3-methylbutyl)(3,3,3-trifluoropropyl)amino]-4-[4-(trifluoromethyl)phenyl]tetrahydro-2H-pyran-2-yl}acetic acid, hydrochloride salt;
{(1R,3S,4R)-4-[(3-methylbutyl)(3,3,3-trifluoropropyl)amino]-3-[4-(trifluoromethyl)phenyl]cyclohexyl}acetic acid, hydrochloride salt;
{(1R,3S,4R)-4-[(3-methylbutyl)(3,3,3-trifluoropropyl)amino]-3-[6-(trifluoromethyl)pyridin-3-yl]cyclohexyl}acetic acid;
{(1R,3S,4R)-4-[(cyclohexylmethyl)(3-methylbutyl)amino]-3-[4-(trifluoromethyl)phenyl]cyclohexyl}acetic acid;
{(1R,3S,4R)-4-{[(1-fluorocyclohexyl)methyl](3-methylbutyl)amino}-3-[4-(trifluoromethyl)phenyl]cyclohexyl}acetic acid;
{(1R,3S,4R)-4-[bis(cyclohexylmethyl)amino]-3-[4-(trifluoromethyl)phenyl]cyclohexyl}acetic acid;
{(1R,3S,4R)-4-[(4,4-difluorocyclohexyl)(3-methylbutyl)amino]-3-[4-(trifluoromethyl)phenyl]cyclohexyl}acetic acid;
{(1R,3S,4R)-4-[(3-methylbutyl)(tetrahydro-2H-pyran-2-ylmethyl)amino]-3-[4-(trifluoromethyl)phenyl]cyclohexyl}acetic acid;
{(2S,5R,6S)-5-[bis(cyclopentylmethyl)amino]-6-[6-(trifluoromethyl)pyridin-3-yl]tetrahydro-2H-pyran-2-yl}acetic acid;
{(2S,5R,6S)-5-[bis(cyclopentylmethyl)amino]-6-[3-(trifluoromethyl)phenyl]tetrahydro-2H-pyran-2-yl}acetic acid;
{(1R,3S,4R)-4-{(cyclopentylmethyl)[(1-methylcyclopentyl)methyl]amino}-3-[4-(trifluoromethyl)phenyl]cyclohexyl}acetic acid;
{(2R,5R,6S)-5-[bis(cyclopentylmethyl)amino]-6-[4-(trifluoromethyl)phenyl]tetrahydro-2H-pyran-2-yl}acetic acid;
{(1R,3S,4R)-4-(4-methyl-2-azaspiro[5.5]undec-2-yl)-3-[4-(trifluoromethyl)phenyl]cyclohexyl}acetic acid;
[(2S,5R,6S)-5-[bis(cyclopentylmethyl)amino]-6-(4-chloro-2-methylphenyl)tetrahydro-2H-pyran-2-yl]acetic acid;
{(1R,3S,4R)-4-[(1R,5S)-3-azabicyclo[3.2.1]oct-3-yl]-3-[4-(trifluoromethyl)phenyl]cyclohexyl}acetic acid;
{(1R,3S,4R)-4-[(cyclopentylmethyl)(4-fluorobenzyl)amino]-3-[4-(trifluoromethyl)phenyl]cyclohexyl}acetic acid;
{(1R,3S,4R)-4-[(cyclopentylmethyl)(2-fluorobenzyl)amino]-3-[4-(trifluoromethyl)phenyl]cyclohexyl}acetic acid;
{(1R,3S,4R)-4-[(cyclopentylmethyl)(3-fluorobenzyl)amino]-3-[4-(trifluoromethyl)phenyl]cyclohexyl}acetic acid, hydrochloride salt;
{(1R,3S,4R)-4-(13-azadispiro[4.1.4.3]tetradec-13-yl)-3-[4-(trifluoromethyl)phenyl]cyclohexyl}acetic acid;
{(1R,3S,4R)-4-[(3-methylbutyl)(tetrahydro-2H-pyran-4-yl)amino]-3-[4-(trifluoromethyl)phenyl]cyclohexyl}acetic acid, ammonium salt;
{(1R,3S,4R)-4-[(cyclohexylmethyl)(tetrahydro-2H-pyran-4-yl)amino]-3-[4-(trifluoromethyl)phenyl]cyclohexyl}acetic acid, ammonium salt;
{(1R,3S,4R)-4-[(2-cyclohexylethyl)(3-methylbutyl)amino]-3-[4-(trifluoromethyl)phenyl]cyclohexyl}acetic acid, trifluoroacetate salt;
{(1R,3S,4R)-4-[(3-methylbutyl)(1,3-thiazol-2-ylmethyl)amino]-3-[4-(trifluoromethyl)phenyl]cyclohexyl}acetic acid, trifluoroacetate salt;
{(1R,3S,4R)-4-[(cyclopentylmethyl)(2-methylbutyl)amino]-3-[4-(trifluoromethyl)phenyl]cyclohexyl}acetic acid, ammonium salt;
{(1R,3S,4R)-4-[(cyclopentylmethyl)(3,3-dimethylbutyl)amino]-3-[4-(trifluoromethyl)phenyl]cyclohexyl}acetic acid, ammonium salt;
{(1R,3S,4R)-4-[(cyclopentylmethyl)(2,2-dimethylpropyl)amino]-3-[4-(trifluoromethyl)phenyl]cyclohexyl}acetic acid, ammonium salt;
{(1R,3S,4R)-4-[(3-cyano-3-methylbutyl)(cyclopentylmethyl)amino]-3-[4-(trifluoromethyl)phenyl]cyclohexyl}acetic acid, trifluoroacetate salt;
{(1R,3S,4R)-4-[(cyclopentylmethyl)(pyrimidin-5-ylmethyl)amino]-3-[4-(trifluoromethyl)phenyl]cyclohexyl}acetic acid, trifluoroacetate salt;
{(1R,3S,4R)-4-{(cyclopentylmethyl)[(1-fluorocyclohexyl)methyl]amino}-3-[4-(trifluoromethyl)phenyl]cyclohexyl}acetic acid, ammonium salt;
{(1R,3S,4R)-4-[(2-cyclohexylethyl)(cyclopentylmethyl)amino]-3-[4-(trifluoromethyl)phenyl]cyclohexyl}acetic acid, trifluoroacetate salt;
{(1R,3S,4R)-4-{(cyclopentylmethyl)[2-(tetrahydro-2H-pyran-2-yl)ethyl]amino}-3-[4-(trifluoromethyl)phenyl]cyclohexyl}acetic acid, trifluoroacetate salt;
{(1R,3S,4R)-4-[(cyclopentylmethyl)(1,3-thiazol-4-ylmethyl)amino]-3-[4-(trifluoromethyl)phenyl]cyclohexyl}acetic acid, trifluoroacetate salt;
{(1R,3S,4R)-4-[(cyclopentylmethyl)(2-ethylbutyl)amino]-3-[4-(trifluoromethyl)phenyl]cyclohexyl}acetic acid, trifluoroacetate salt;
{(1R,3S,4R)-4-[(cyclopentylmethyl)(isobutyl)amino]-3-[4-(trifluoromethyl)phenyl]cyclohexyl}acetic acid, trifluoroacetate salt;
{(1R,3S,4R)-4-[(cyclopentylmethyl)(3,3,3-trifluoropropyl)amino]-3-[4-(trifluoromethyl)phenyl]cyclohexyl}acetic acid, trifluoroacetate salt;
[(2S,5R,6S)-5-[bis(cyclopentylmethyl)amino]-6-(4-cyanophenyl)tetrahydro-2H-pyran-2-yl]acetic acid, ammonium salt;
[(2S,5R,6S)-5-[bis(cyclopentylmethyl)amino]-6-(3-chlorophenyl)tetrahydro-2H-pyran-2-yl]acetic acid, ammonium salt;
{(1R,3S,4R)-3-(4-chlorophenyl)-4-[(cyclopentylmethyl)(3,3,3-trifluoropropyl)amino]cyclohexyl}acetic acid, trifluoroacetate salt;
{(1R,3S,4R)-3-(4-chlorophenyl)-4-[(cyclopentylmethyl)(2-ethylbutyl)amino]cyclohexyl}acetic acid, ammonium salt;
{(1R,3S,4R)-4-[(cyclobutylmethyl)(cyclopentylmethyl)amino]-3-[4-(trifluoromethyl)phenyl]cyclohexyl}acetic acid, ammonium salt
{(1R,3S,4R)-3-(4-chlorophenyl)-4-[(cyclopentylmethyl)(3,3,3-trifluoro-2-methylpropyl)amino]cyclohexyl}acetic acid, trifluoroacetate salt;
{(1R,3S,4R)-4-{[(1-fluorocyclohexyl)methyl](isobutyl)amino}-3-[4-(trifluoromethyl)phenyl]cyclohexyl}acetic acid, trifluoroacetate salt;
{(1R,3S,4R)-4-{(cyclopentylmethyl)[(2-methyl-1,3-oxazol-4-yl)methyl]amino}-3-[4-(trifluoromethyl)phenyl]cyclohexyl}acetic acid, trifluoroacetate salt;
[(2S,5R,6S)-5-[bis(cyclopentylmethyl)amino]-6-(4-chloro-3-fluorophenyl)tetrahydro-2H-pyran-2-yl]acetic acid, ammonium salt;
[(2S,5R,6S)-5-[bis(cyclopentylmethyl)amino]-6-(4-chlorophenyl)tetrahydro-2H-pyran-2-yl]acetic acid;
{(1R,3S,4R)-4-{(cyclopentylmethyl)[(1S)-1,3-dimethylbutyl]amino}-3-[4-(trifluoromethyl)phenyl]cyclohexyl}acetic acid;
{(1R,3S,4R)-4-[(4-ethoxybenzyl)(isobutyl)amino]-3-[4-(trifluoromethyl)phenyl]cyclohexyl}acetic acid, trifluoroacetate salt;
{(1R,3S,4R)-4-{[(1-fluorocyclohexyl)methyl](2-fluoro-6-methoxybenzyl)amino}-3-[4-(trifluoromethyl)phenyl]cyclohexyl}acetic acid, trifluoroacetate salt;
{(1R,3S,4R)-4-{isobutyl[(3-propylisothiazol-4-yl)methyl]amino}-3-[4-(trifluoromethyl)phenyl]cyclohexyl}acetic acid, trifluoroacetate salt;
{(1R,3S,4R)-4-[(cyclohex-1-en-1-ylmethyl)(isobutyl)amino]-3-[4-(trifluoromethyl)phenyl]cyclohexyl}acetic acid, trifluoroacetate salt;
{(1R,3S,4R)-4-{(2,3-dihydro-1-benzofuran-5-ylmethyl)[(1-fluorocyclohexyl)methyl]amino}-3-[4-(trifluoromethyl)phenyl]cyclohexyl}acetic acid, trifluoroacetate salt;
{(1R,3S,4R)-4-{(2H-chromen-3-ylmethyl)[(1-fluorocyclohexyl)methyl]amino}-3-[4-(trifluoromethyl)phenyl]cyclohexyl}acetic acid, trifluoroacetate salt;
{(1R,3S,4R)-4-{[(6,6-dimethylbicyclo[3.1.1]hept-2-en-2-yl)methyl](isobutyl)amino}-3-[4-(trifluoromethyl)phenyl]cyclohexyl}acetic acid, trifluoroacetate salt;
{(1R,3S,4R)-4-{[(4-ethyl-1,3-thiazol-2-yl)methyl][(1-fluorocyclohexyl)methyl]amino}-3-[4-(trifluoromethyl)phenyl]cyclohexyl}acetic acid, trifluoroacetate salt;
{(1R,3S,4R)-4-{(2,1,3-benzoxadiazol-5-ylmethyl)[(1-fluorocyclohexyl)methyl]amino}-3-[4-(trifluoromethyl)phenyl]cyclohexyl}acetic acid, trifluoroacetate salt;
{(1R,3S,4R)-4-[(4-tert-butylbenzyl)(isobutyl)amino]-3-[4-(trifluoromethyl)phenyl]cyclohexyl}acetic acid, trifluoroacetate salt; or
{(1R,3S,4R)-4-{[(4,5-dimethyl-2-furyl)methyl](isobutyl)amino}-3-[4-(trifluoromethyl)phenyl]cyclohexyl}acetic acid, trifluoroacetate salt.
or a pharamaceutically acceptable salt thereof.

15. A method for the treatment of a disease or condition selected from the group consisting of neurological and psychiatric disorders comprising administering to the mammal an effective amount of a compound of claim 1 or 14 or pharmaceutically acceptable salt thereof.

16. A pharmaceutical composition comprising a compound of claim 1 or 14 or a pharmaceutically acceptable salt thereof and a pharmaceutically acceptable carrier.
